(19) United States
(12) Patent Application Publication
(10) Pub. No.: US 2023/0227506 A1
(43) Pub. Date: Jul. 20, 2023

(54) Fatty Acid Complexes Of Coronavirus Spike Protein And Their Use

(71) Applicants: THE UNIVERSITY OF BRISTOL, Bristol (GB); GBIOTECH S.À.R.L., Geneva (CH)

(72) Inventors: Daniel Joseph Fitzgerald, Carouge (CH); Imre Berger, Bristol (GB); Christiane Berger-Schaffitzel, Bristol (GB); Christine Toelzer, Bristol (GB); Kapil Gupta, Bristol (GB)

(21) Appl. No.: 18/002,461

(22) PCT Filed: Jun. 18, 2021
(86) PCT No.: PCT/EP2021/066723

§ 371 Date: Dec. 19, 2022

(30) Foreign Application Priority Data
Jun. 18, 2020 (EP) 20180964.7
Mar. 16, 2021 (EP) 21163016.5
Apr. 21, 2021 (EP) 21170570.2

(51) Int. Cl. C07K 14/005; A61K 31/201; A61K 9/00; A61P 31/14; C07K 16/10; G01N 33/569

(52) U.S. Cl. CPC C07K 14/005; A61K 31/201; A61K 9/0073; A61P 31/14; C07K 16/10; G01N 33/56983; G01N 2333/165; G01N 2500/04

(57) ABSTRACT

Complexes of coronavirus spike proteins, as well as fragments or mutants thereof wherein the fragment or mutant thereof at least contains a receptor binding domain of said coronavirus spike protein, with linoleic acid, or a derivative or a salt or a mimetic thereof. Methods for producing the complexes of the invention by incubating coronavirus spike proteins with linoleic acid or a derivative or a salt or a mimetic thereof. . In vitro methods for identifying molecules which have therapeutic potential for diseases caused by coronaviruses by contacting the molecule with a coronavirus spike protein and linoleic acid or a derivative or salt or mimetic thereof. A method of treatment of coronavirus infection by administration of linoleic acid, or a derivative, a salt or a mimetic thereof to a subject in need thereof, by administration of an aerosol formulation or dry powder formulation to the respiratory tract, preferably by nasal administration.

### FIELD OF THE INVENTION

The present invention relates to complexes of coronavirus spike proteins as well as fragments or mutants thereof wherein the fragment or mutant thereof at least contains a receptor binding domain of said coronavirus spike protein, with linoleic acid, a derivative or a salt or a mimetic thereof. Further subject matter of the invention relates to methods for producing the complexes of the invention. A further aspect of the invention is in vitro methods for identifying molecules which have therapeutic potential for diseases caused by coronaviruses. A further aspect of the invention is therapeutics to treat coronavirus infection by administration of linoleic acid, a derivative, a salt or a mimetic thereof to a subject in need thereof, and diagnostics to detect coronavirus infection.

### REFERENCE TO SEQUENCE LISTING

This application contains a Sequence Listing. The application thus incorporates by reference the material in the ASCII text file EFS3544151.txt, created on Dec. 19, 2022, and having a size of 100,908 bytes.

### BACKGROUND OF THE INVENTION

The present invention is, at least in part, based on the following background:

SARS-CoV-2 and other human coronaviruses. Key to SARS-CoV-2 therapeutic development is a better understanding of the mechanisms which drive its high infectivity, unusually broad tissue tropism and severe pathology (1,2,3). At present, there are seven coronaviruses that are known to infect humans. The four endemic human coronaviruses OC43, 229E, HKU1, and NL63 cause mild, self-limiting upper respiratory tract infections while pandemic virus SARS-CoV-2, and earlier SARS-CoV and MERS-CoV can cause severe pneumonia with acute respiratory distress syndrome, multi-organ failure, and death (3,5). In order to enable development of effective therapeutic interventions, a central goal of ongoing research into the COVID-19 pandemic is to determine the features of SARS-CoV-2 that distinguish it from predecessor coronaviruses and provide it with a lethal combination of high infectivity and high pathogenicity.

SARS-CoV-2 pathology. COVID-19 patients display an odd collection of symptoms not seen with any previous human coronavirus including blood clots, strokes, “COVID toes” and heart attacks (6,7,8). While SARS-CoV did not significantly spread past the lungs, a recent study reported damage or severe inflammation in SARS-CoV-2 patients' endothelial cells in the heart, kidneys, liver, and intestines, suggestive of a vascular infection rather than a respiratory disease (1). While this significantly expanded tissue tropism might be partially explained by more widespread and effective spike glycoprotein processing systems (9,10,11), the accompanying severe immune dysregulation, inflammation and tissue pathology both inside and outside of the lungs remains poorly understood.

Receptor recognition and cell entry by SARS-CoV-2. Receptor recognition by coronaviruses is an important determinant of viral infectivity and pathogenesis, and represents a major target for antiviral therapeutic development (12). The attachment of SARS-CoV-2 to a host cell is initiated by interactions between the spike (S) glycoprotein and its cognate receptor angiotensin-converting enzyme 2 (ACE2) which are higher affinity than with previous closely related SARS-CoV and also other human coronaviruses (9,13,14). Following receptor docking by SARS-CoV-2 and prior to membrane fusion, the S glycoprotein can be processed by a plasma membrane—associated protease, TMPRSS2, which helps to unload virus components into the host cell cytoplasm (9,10). Once inside the host cell, human coronaviruses including the mild endemic species have evolved systems to evade the innate immune system (15,16) and remodel its lipid metabolism to facilitate virus replication (4). SARS-CoV-2 has acquired additional novel functions that characterize its harsh disease phenotype. Relative to other human coronaviruses SARS-CoV-2 exhibits more effective protease processing (10,11), and a broader cell tropism to drive rapid unloading of virus into diverse tissues (1,17). A novel S1/S2 polybasic furin protease cleavage site stimulates cell-cell fusion and entry into host cells (11).

Dysregulated immune response and inflammation triggered by SARS-CoV-2 infection. Infection by SARS-CoV-2 also triggers an unusually impaired and dysregulated immune response (18) and a heightened inflammatory response (2). Hyper-immunity and inflammatory responses of the host to SARS-CoV-2 work in synergy with interferon production in the vicinity of infected cells to drive a feed-forward loop to upregulate ACE2 and further escalate infection (19).

Structure of the SARS-CoV-2 S glycoprotein as visualized by cryo-EM. In the search for additional novel functions that contribute to the observed extreme pathology of infection, we determined the structure of the SARS-CoV-2 S glycoprotein by cryo-EM and discovered in our structure that each S in the trimer tightly binds one copy of Linoleic Acid. This has not been previously disclosed in any crystal structures or cryo-EM structures of coronavirus S proteins. Linoleic Acid (hereinafter referred to as “LA”) is a polyunsaturated omega-6 fatty acid and is one of two essential fatty acids for humans, who must obtain it through their diet. It is a colorless or white oil that is virtually insoluble in water.

Via analysis of available crystal structures and cryo-EM structures, we found four molecular features mediating LA binding to SARS-CoV-2, SARS-CoV and MERS-CoV S proteins: i) a conserved hydrophobic pocket; ii) a gating helix; iii) amino acid residues pre-positioned to interact with the LA carboxy headgroup; and iv) loosely packed RBDs in the ‘apo’ form (where ‘apo’ means S proteins without bound LA). On the other hand, in each of the four common endemic human coronaviruses, one or more of these four architectural prerequisites mediating LA binding are lacking in the S protein structures. Furthermore, we demonstrated via cryo-EM that conformational changes in the RBD trimer triggered by LA binding impact ACE2 docking and therefore probably infectivity. The S protein's highly selective binding of LA originates from the very well-defined size and shape complementarity afforded by the LA-binding pocket, which is supported by the observation that we only detected LA in our analyses, and not other free fatty acids, even though hundreds of distinct free fatty acids (FFAs) were present in the culture media and the cultured cells. The LA-binding pocket thus presents a promising target for future development of small molecule or biologics that, for example, could irreversibly lock S in the closed conformation and thereby interfere with binding of a LA-containing coronavirus with its target receptor which, for example, could degrade infectivity.

It is therefore one object of this invention to provide material compositions and methods which enable discovery, i.e. identification of small molecule or biologic drug candidates targeting the LA-binding pocket which inhibit the binding of LA to the coronavirus spike protein (hereinafter also referred to as “coronavirus S protein” or simply “S protein”). It is a second object of this invention to provide material compositions and methods which enable discovery, i.e. identification, of small molecule or biologic drug candidates targeting the LA-binding pocket which are inhibitors of the binding of a coronavirus with its target receptor.

Biological implications of LA binding to SARS-CoV-2 S protein. A recent proteomic and metabolomic study of COVID-19 patient sera evidenced continuous decrease of FFAs including LA (20). Lipid metabolome remodeling is a common element of viral infection (21,22), including for the baculovirus (23) used here to express S protein. For coronaviruses, the LA to arachidonic acid (AA) metabolism axis was identified as the epicenter of lipid remodeling (4).

It is therefore a third object of this invention to provide material compositions and methods which enable discovery, i.e. identification, of small molecule or biologic drug candidates associated with LA binding by coronavirus S protein, which inhibit lipid metabolome remodeling triggered by coronavirus infection. Interestingly, exogenous supplement of LA or AA suppressed virus replication (4). It is remarkable in this context that the S trimer of SARS-CoV-2 binds LA with astonishing specificity, endowing a scavenger function on S, poised to impact lipid remodeling and fuel SARS-CoV-2 pathologies. Lipid metabolome remodeling alters three separate processes in cells (4,21,22): (i) energy homeostasis via changes in catabolic and anabolic precursor equilibria; (ii) fluidity and elasticity of biological membranes, via changes in e.g. saturated/unsaturated fatty acid ratio in phospholipids; and (iii) cell signaling, via changes in levels of lipid-based cell signaling precursors. Regarding the potential impact of LA axis remodeling on fluidity and elasticity of biological membranes, we note that the FFA composition of phospholipid bilayers is a key element in maintaining surface tension in lungs, and alteration of LA axis lipid composition is observed in acute respiratory distress syndrome and severe pneumonia, both of which are key symptoms of SARS-CoV-2 infection.

It is a fourth object of this invention to provide material compositions and methods which enable discovery, i.e. identification, of small molecule or biologic drug candidates associated with LA binding by coronavirus S protein, which modulate or stabilize the fluidity and elasticity of biological membranes in coronavirus infected cells.

Significant changes in cell signalling are anticipated due to LA to AA metabolome axis remodelling, since the LA biosynthetic pathway leads to eicosanoids, which are prominent signalling molecules involved in inflammatory processes (24). The present invention reveals that SARS-CoV-2 comprises a FFA-binding pocket that specifically accretes LA, and suggests that this could be a feature shared with SARS-CoV and MERS-CoV. The high affinity, high specificity LA scavenger function conveyed by our results could confer a tissue-independent mechanism by which pathogenic coronavirus infection drives immune dysregulation and inflammation. Our findings also suggest that the multinodal LA signalling axis also represents excellent therapeutic intervention points against coronavirus infections, particularly in patient groups with increased risk due to metabolic preconditions, such as diabetes, or dislipidemias. It is a fifth object of this invention to provide material compositions and methods which enable discovery, i.e. identification, of small molecule or biologic drug candidates (together “candidate molecules”) associated with LA binding by coronavirus S protein, which modulate or stabilize cell signalling along the LA to AA (arachidonic acid) metabolome axis in coronavirus infected cells.

Fatty acids have been suggested in the prior art for treatment of infection by enveloped viruses. WO 94/16061 Al discloses a method of inactivating enveloped viruses by the use of paucilamellar lipid vesicles, preferably made of non-phospholipids, which fuse with the enveloped virus whereafter the viral nucleic acid denature. In a similar direction, US 2015/0065458 discloses the use of various non-phospholipid present in compositions in the form of vesicles for prevention of diseases caused by enveloped viruses, including coronaviruses, and further reports that the effect of the non-phospholipid lipid vesicles can be strongly improved by co-incubation of virus-infected cells with cyclodextrin.

It is a fifth object to provide improved compositions, delivery devices and methods for treatment and/or prevention of coronavirus infections, in particular caused by SARS-CoV, MERS and/or SARS-CoV-2, and disease states relating to such coronavirus infections such as COVID-19.

### SUMMARY OF THE INVENTION

The present invention provides, under a first aspect, an isolated complex of a coronavirus S protein or a fragment or mutant thereof wherein said fragment or mutant at least contains a receptor binding domain of said coronavirus S protein, with LA (linoleic acid, 18:2 (n-6)), a derivative, a salt or a mimetic thereof.

A “derivative” or “mimetic” of LA is generally a structure having the necessary requirements for binding to the coronavirus S protein or a fragment or mutant thereof as disclosed herein, in particular to a binding pocket of the coronavirus S protein or a fragment or mutant thereof as disclosed herein, combining at least the following structural features: at least two, preferably exactly two, basic amino acid residues, typically being directly following one another in the amino acid sequence of said coronavirus S protein or a fragment or mutant thereof as defined herein, and a tube-like cavity in the structure of said coronavirus S protein or a fragment or mutant thereof as disclosed herein formed by lipophilic acid residues. Preferably, the binding site is also composed by an alpha-helix gating said hydrophilic tube. Typical derivatives or mimetics of LA have a polar head group and an apolar extended tail group, wherein the polar head group is coordinated by said at least two, or exactly two, basic amino acid residues, and said apolar tail group fits into said hydrophilic tube.

More preferably, a derivative or mimetic of LA is characterised by the following general formula (I):

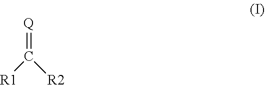

wherein

Q is selected from 0, S and NH, and is most preferred O;

R1 is selected from OR, NHR3, and SH, wherein R3 is H or a short chain alkyl or substituted alkyl group having 1 to 3 carbon atoms, and is preferably NH2 or OH, most preferred OH; and

R2 is a straight unsubstituted or substituted hydrocarbyl group having from 13 to 21 C atoms, preferably 17 C atoms, optionally linked or bound to a detectable label.

From the present disclosure, the skilled person is aware that the expression “a derivative or mimetic of LA” according to the invention as also includes other fatty acids besides LA itself.

Preferably, the group R2 has at least one unsaturated C-C bond, more preferably two unsaturated C—C bonds. Preferably, the group R2 has 1, 2, 3, 4 or 5 unsaturated C—C bonds. More preferably, at least one unsaturated C—C bonds is between C-8 and C-9. In other preferred embodiments, at least one unsaturated C—C bond C-11 and C-12 of the hydrocarbyl group. Most preferred the R2 has an unsaturated C—C bond between C-8 and C-9 and an unsaturated C-C-bond between C-11 and C-12. It is to be understood that the above C numbering of the hydrocarbyl group is counted from the carbon bound to the C=Q group in formula (I). Preferably, the one or more unsaturated C—C— bond(s) in the hydrocarbyl groups is/are C—C double bonds.

Preferred embodiments of compounds of formula (I) (in particular fatty acids) are outlined as follows (indicated by trivial name(s)) and according to the lipid number and omega-x nomenclature):

An especially preferred compound (fatty acid of formula (I)) is oleic acid (18:1 cis-9).

Another preferred compound (fatty acid of formula (I)) is arachidonic acid (20:4 (n-6); also denoted as “AA” or “ARA”).

Other preferred embodiments of a compound (fatty acid of formula (I)) include elaidic acid (18:1 trans-9), eicosapentaenoic acid (20:5 (n-3), stearic acid (18:0), gamma-linoleic acid (also denoted as “GLA”; 18:3 (n-6)), calendic acid (18:3 (n-6)), arachidic acid (synonym: eicosanoic acid; 20:0), and dihomo-gamma-linoleic acid (20:3 (n-6)).

Still other preferred embodiments of a compound (fatty acid of formula (I)) include docosadienoic acid (22:2 (n-6)), adrenic acid (22:4 (n-6)), palmitic acid (16:0) and behenic acid (synonym: docosanoic acid; 22:0).

In preferred embodiments of the present invention, the compound according to formula (I) is a free fatty acid, with the above embodiments of fatty acids being preferred fatty acids according to the invention or for use according to the invention, respectively.

According to the invention, a compound of formula (I) such as preferred compounds as outlined above includes salts, anions and conjugates of such compounds (or fatty acids, respectively) as defined herein.

A “salt” of LA or generally a “salt” of a compound of formula (I), respectively, is typically an LA salt or generally a salt of a compound of formula (I), respectively, selected from the alkali metal and earth alkaline metals of the periodic system. Preferred alkaline salts of LA or generally preferred salts of a compound of formula (I), respectively, are those of sodium, kalium and lithium. Preferred earth alkaline salts of LA or generally preferred earth alkaline salts of a compound of formula (I), respectively, are those of calcium and barium. It is to be understood that the “salts” of a compound of formula (I) are preferably salts of preferred fatty acid compounds as outlined above.

A “salt” of LA according to the invention may be also or alternatively used according to the invention to refer to the anionic form of LA or its mimetics or derivatives, in particular compounds (in particular fatty acids) of formula (I), preferably those preferred embodiments as outlined above.

The present invention also relates to a composition of complexes as defined herein wherein the complexes are composed of the coronavirus S protein or fragment of mutant thereof and two or more different fatty acids, in particular LA and one or more compounds of formula (I), preferably one or more of the preferred embodiments of compounds of formula (I), more preferably LA and oleic acid, further preferred oleic acid and arachidonic acid, still further preferred LA and arachidonic acid, yet further preferred LA, oleic acid and arachidonic acid, each optionally further containing complexes of the coronavirus S protein or fragment of mutant thereof with other compounds of formula (I), preferably other preferred compounds of formula (I) as outlined above.

The complex of the invention may comprise, or is composed of, respectively, 1, 2 or 3 copies of said coronavirus S protein or fragment or mutant thereof, whereby it is understood that 2 copies represents a dimer of said coronavirus S protein or fragment or mutant thereof, and 3 copies represents a trimer of said coronavirus S protein or fragment or mutant thereof. Preferably, the complex is composed of a dimer or trimer of said coronavirus S protein or fragment or mutant thereof. The complex of the invention may likewise contain 1, 2 or 3 LA molecules (or derivatives, salts or mimetics thereof). In specifically preferred embodiments, the complex comprises, or is composed of, respectively, 3 copies of the S protein or fragment or mutant thereof. In further preferred embodiments, the complex comprises 2 or 3, preferably 3, LA molecules (or derivatives, salts or mimetics thereof). More preferably, the complex comprises, or is composed of, respectively, 3 copies of the S protein or fragment or mutant thereof and 3 LA molecules (or derivatives, salts or mimetics thereof). In further preferred embodiments of the invention, the complex comprises or is composed of, respectively, a dimer of said and contains 1 or 2, preferably 2 LA molecules (or derivatives, salts or mimetics thereof). It is to be understood that such complexes of the invention comprising or being composed of, respectively, 2 or more such as 2 or 3 copies of said coronavirus S protein or fragment or mutant thereof, may contain a combination of LA and other compound(s) of formula (I) and/or a combination of 2 or more compounds of formula (I), whereby such multiple copy complexes may comprise 2 or more, more preferably 2 or 3, copies of the coronavirus S protein or fragments or mutants thereof in complex with two or more, preferably two or three different fatty acids, in particular LA and 1 or 2 compounds of formula (I), preferably 1 or 2 of the preferred embodiments of compounds of formula (I), more preferably LA and oleic acid, further preferred oleic acid and arachidonic acid, still further preferred LA and arachidonic acid, yet further preferred LA, oleic acid and arachidonic acid.

In preferred embodiments of the complex according to the invention, the coronavirus S protein or fragment or mutant thereof is selected from S proteins or fragments or mutants thereof of a coronavirus causing respiratory disease, more preferred pneumonia. In preferred embodiments, the coronavirus S protein or fragment or mutant thereof is derived from a coronavirus that infects humans.

Preferably, the coronavirus S protein or fragment thereof is a S protein or fragment or mutant thereof of a coronavirus selected from the group consisting of SARS-CoV, MERS-CoV and SARS-CoV-2, whereby SARS-CoV-2 is particularly preferred.

Especially preferred examples of coronavirus S proteins have an amino acid sequence selected from amino acid sequences of SEQ ID NO: 1 to SEQ ID NO: 15, whereby SEQ ID NO: 1 is a preferred example for a fragment representing the ectodomain of a coronavirus S protein. In that case the ectodomain has a C terminal His tag. SEQ ID NOs: 2 to 8 represent full length coronavirus S proteins, and SEQ ID NOs: 9 to 15 represent preferred receptor binding domains (RBDs) of coronavirus S proteins. The amino acid sequences of the afore-mentioned constructs and information from which coronavirus they are derived are given further below. Highly preferred coronavirus S proteins for use in the invention are selected from SEQ ID NO: 1, 2, 3, and 4, with SEQ ID NO: 1 and 2 being most preferred. Particularly preferred fragments of coronavirus S proteins, namely, receptor binding domains, for use in the invention are selected from 9, 10, and 11, with SEQ ID NO: 9 being most preferred. Further preferred examples of coronavirus S proteins or fragment or mutants thereof comprise an amino acid sequences as defined in SEQ ID NO:16 or SEQ ID NO: 17 or SEQ ID NO:18.

The coronavirus S protein, including fragments retaining the structure for binding LA or a salt or a derivative or a mimetic thereof, preferably a receptor binding domain of said coronavirus S protein, can contain one or more mutations in comparison to the wild-type sequence of the protein or fragment thereof. Such mutants may differ from the natural occurring, i.e. wild-type, coronavirus S protein (or a certain fragment of such a naturally occurring coronavirus S protein) such as an amino sequence selected from the sequences according to SEQ ID NO: 1, 2, 3, 4, 5, 6, 7, 8, 9, 10, 11, 12, 13, 14, 15, 16, 17, or 18, preferably SEQ ID NO: 2, 3, or 4, further preferred SEQ ID NO: 9, 10 , 11, by one or more amino acid deletions, additions, and/or substitutions, with the proviso that the resulting mutant retains the necessary structure for binding LA (or salt or derivative or mimetic of LA), preferably the mutant contains at least the receptor binding domain of the coronavirus S protein or the fragment thereof. The mutant for use in the invention has typically at least 90%, preferably at least 95%, more preferably at least 96%, even more preferred at least 97%, still further preferred at least 98%, most preferred at least 99% sequence homology with the amino acid sequence of the wild-type coronavirus S protein or fragment thereof.

Mutants and fragments of coronavirus S proteins contained complexes of the invention preferably comprise a receptor binding domain but lack a transmembrane domain, more preferably a trimerization domain of a coronavirus S protein. Coronavirus S protein derived constructs for the complexes of the invention preferably lack the transmembrane portion, while preferably retaining the trimerization portion, are particularly beneficial for using the constructs, including when in complex with LA or a salt or a derivative or a mimetic thereof, in aqueous solution, preferably for the assay methods as disclosed and described further below. In other embodiments, the fragment or mutant, respectively of the coronavirus S protein does not contain its transmembrane domain nor its wild-type trimerization domain or portion, respectively. Where fragments of mutants of coronavirus S protein do not contain a wild-type trimerization domain, it is preferred that a mutation or linker is provided which regains the trimerization function. For preferred embodiments of the invention, where the inventive complex comprises 3 copies of the fragment or mutant of the coronavirus S protein, it is preferred that the mutant lacking the natural sequences for trimerization has at least one mutation that enables the trimerization of the mutant or fragment. This can, e.g. be accomplished by coupling a non-transmembrane, in particular soluble, i.e. soluble in water or aqueous solutions, trimerization domain to the mutant or fragment construct. Other means for providing the mutant or fragment include introduction of amino acid mutations, which can be additions or, preferably, substitutions, of an amino acid in the wild-type sequence. Another possibility is to include non-wild type trimerization domain, e.g. a hydrophilic trimerization domain from phage T4, or heptad repeat sequence forming an alpha helical coiled coil structure. Corresponding heptad repeat sequences are known to the skilled person.

In a more general aspect of the invention, preferably based on complexes comprising or composed of, respectively, coronavirus S proteins or fragment of mutants thereof as defined herein from SARS-CoV or SARS-CoV-2, preferably SARS-CoV-2, the complex of the invention is characterised in that the binding site for the molecule, preferably LA or a derivative or mimetic or salt thereof as disclosed herein, is at least provided by, or contains or consists of, an amino acid sequence comprising the following consensus sequence (from N- to C-terminal):

(SEQ ID NO: 16)
X₁X₂Z₁X₃Z₂X₄Z₃X₅X₆Z₄Z₅X₇X₈X₉X₁₀X₁₁Z₆X₁₂X₁₃X₁₄
X₁₅X₁₆X₁₇X₁₈X₁₉Z₇X₂₀X₂₁Z₈X₂₂Z₉X₂₃YX₂₄X₂₅Z₁₀Y
X₂₆X₂₇X₂₈X₂₉Z₁₁X₃₀X₃₁Z₁₂X₃₂Z₁₃X₃₃X₃₄X₃₅X₃₆X₃₇
X₃₈X₃₉Z₁₄X₄₀X₄₁X₄₂X₄₃Z₁₅X₄₄X₄₅X₄₆X₄₇X₄₃X₄₉X₅₀
X₅₁X₅₂X₅₃X₅₄X₅₅X₅₆X₅₇X₅₈RQX₅₉

wherein:

each X₁ to X₅₉ is independently any amino acid;

each Z₁ to Z₁₅ is independently an amino acid having a hydrophobic side chain, preferably selected from C, F, V, I, A, and L.

Amino acids in the above SEQ ID NO: 16 not being X or Z are amino acid represented by the single letter code of amino acids.

In this complex of the invention defined by an coronavirus S protein, fragment or mutant thereof as defined herein according to above SEQ ID NO: 16, the complex is may formed with a molecule having a structure binding into a binding site formed at least in part by the above SEQ ID NO: 16, preferably a fatty acid or fatty acid derivative or fatty acid salt or fatty acid anion or fatty acid mimetic, more preferably LA or a salt or a derivative or mimetic thereof as defined above.

In particularly preferred embodiments of the invention, the coronavirus S protein or mutant or fragment thereof comprises a fatty acid, preferably LA, binding site (also referred to herein as “binding pocket”) comprises an amino acid sequence selected from SEQ ID NO: 17 or 18:

(SEQ ID NO: 17)
NLCPFGEVFNATRFASVYAWNRKRISNCVADYSVLYNSA
SFSTFKCYGVSPTKLNDLCFTNVYADSFVIRGDEVRQI
(SEQ ID NO: 18)
NLCPFGEVFNATKFPSVYAWERKKISNCVADYSVLYNST
FFSTFKCYGVSATKLNDLCFSNVYADSFVVKGDDVRQI

For detecting binding of LA (or derivative or mimetic or salt thereof) to the coronavirus S protein (or fragment or mutant thereof as defined herein), it is preferred that the LA or salt or derivative or mimetic thereof, in particular when present in the inventive complex, comprises a detectable label. The LA or salt or derivative or mimetic thereof is particularly useful in the methods for producing the inventive complex as further described below. Likewise, the coronavirus mutant or fragment thereof as disclosed herein may contain a detectable label.

Labels for use in the invention include any chemical group or atom which can be detected by physical or chemical means. Detectable labels for use in the invention include, but are not limited to, radioactive labels, dyes, fluorophores, labels detectable by chemical reactions such as enzymatic reactions. Chemical groups for detection according to the invention can be introduced by methods generally known to the skilled person.

Also for employment of complexes of the invention in assay methods, preferably methods disclosed herein, the complex is immobilized, more preferably on a surface of a test device.

In preferred embodiments of the invention the complex is bound to a receptor for the coronavirus S protein or fragment or mutant thereof. In particular for analytical applications, the receptor is preferably immobilized, more preferably on a surface of a test device. The receptor for the coronavirus S protein is preferably the natural receptor for the respective coronavirus to which the coronavirus S protein binds upon infection of the species, in particular humans. A preferred receptor for use in the invention is angiotensin converting enzyme 2 (ACE2).

Coupling techniques for immobilizing the complex or the receptor are generally known in the art and preferably include immobilization via natural binding partners such as coupling via biotin/streptavidin or similar binding partners. In such cases the complex, preferably the coronavirus S protein or mutant or fragment thereof as defined herein, or the receptor is coupled to one the respective binding partner, e.g. biotin.

Test devices according to the invention, in particular for providing the surface for immobilization, include surfaces of test tubes, analytical plates such as microplates, e.g. ELISA plates, beads of polymeric material and the like.

Further subject matter of the invention are various methods for producing the complex of the invention.

In one embodiment, the method for producing a complex as defined herein comprises the step of incubating, preferably in an aqueous solution, more preferably a buffered aqueous solution, an isolated coronavirus S protein or fragment or mutant thereof as disclosed herein with linoleic acid or a derivative or a salt or a mimetic thereof.

Further methods for producing the complex of the invention are preferably based on recombinant technologies.

In an embodiment, a method for producing a complex of the invention comprises the step of expressing the coronavirus S protein or a fragment or mutant thereof as defined herein in a recombinant host cell in the presence of linoleic acid or a salt or a derivative or a mimetic thereof, and, optionally purifying the complex from the host cell.

A further recombinant embodiment is a method for producing a complex of the invention comprising the steps of:
(i) introducing into host cells a heterologous nucleic acid encoding the coronavirus S protein or a fragment or mutant thereof wherein said fragment or mutant at least contains the receptor binding domain of said coronavirus S protein;
(ii) culturing said host cells in the presence of linoleic acid; and, optionally
(iii) purifying the complex from the host cells and/or the culture medium.

In another embodiment, the recombinant method for producing a complex of the invention comprises the steps of:
(1) expressing the coronavirus S protein or a fragment or mutant thereof wherein the fragment or mutant at least contains the receptor binding domain of said coronavirus S protein, in a recombinant host cell, preferably a linoleic acid-free host cell and medium;
(2) isolating the expressed coronavirus S protein or a fragment or mutant thereof from the host cell; and
(3) incubating the isolated coronavirus S protein or fragment or mutant thereof with linoleic acid or a derivative or a salt or a mimetic thereof,

The isolated coronavirus S protein or fragment or mutant thereof is preferably immobilized on the surface of a detection device, e.g. on a surface of a test device as described above, before step (3).

Host cells for use in the invention may be prokaryotic or eukaryotic. Eukaryotic host cells may for example be mammalian cells, preferably human cells. Examples of human host cells include, but are not limited to, HeLa, Huh7, HEK293, HepG2, KATO-III, IMR32, MT-2, pancreatic β-cells, keratinocytes, bone-marrow fibroblasts, CHP212, primary neural cells, W12, SK-N-MC, Saos-2, WI38, primary hepatocytes, FLC4, 143TK, DLD-1, embryonic lung fibroblasts, primary foreskin fibroblasts, MRCS, and MG63 cells. Further preferred host cells of the present invention are porcine cells, preferably CPK, FS-13, PK-15 cells, bovine cells, preferably MDB, BT cells, bovine cells, such as FLL-YFT cells. Other eukaryotic cells useful in the context of the present invention are C. elegans cells. Further eukaryotic cells include yeast cells such as S. cerevisiae, S. pombe, C. albicans and P. pastoris. Furthermore, the present invention is directed to insect cells as host cells which include cells from S. frugiperda, more preferably Sf9, Sf21, Express Sf+, High Five H5 cells, and cells from D. melanogaster, particularly S2 Schneider cells. Further host cells include Dictyostelium discoideum cells and cells from parasites such as Leishmania spec.

Prokaryotic hosts according to the present invention include bacteria, in particular E. coli such as commercially available strains like TOP10, DH5α, HB101 etc.

For expressing the coronavirus S protein or fragment or mutant thereof a heterologous nucleic acid encoding the coronavirus S protein or fragment or mutant as described herein is typically introduced into said host cells (e. g, and preferably, as defined in above step (i) of one embodiment of the inventive production methods). For expressing the coronavirus S protein or fragment of mutant thereof, the heterologous nucleic acid is typically a vector comprising the nucleotide sequence encoding the coronavirus S protein or fragment of mutant thereof, preferably in an expression cassette.

The person skilled in the art is readily able to select appropriate vector construct/host cell pairs for appropriate propagation and/or transfer of the heterologous nucleic acid for use in the production methods according to the present invention into a suitable host. Specific methods for introducing appropriate vector elements and vectors into appropriate host cells are equally known to the art and methods can be found in the latest edition of Ausubel et al. (ed.) Current Protocols In Molecular Biology, John Wiley & Sons, New York, USA.

Preferred systems for expression of the coronavirus S protein or fragment or mutant thereof as defined herein are baculoviral expression systems. A particularly preferred baculovirus expression system is MultiBac (see WO-A-2005/085456 A1) which is commercially available from Geneva Biotech, Geneva, Switzerland.

Further subject matter of the invention are methods for transforming a coronavirus S protein or a fragment or mutant thereof as disclosed herein from an Apo form (also referred to as an Apo state) to a Holo form (also referred to as a Holo state) and vice versa.

One embodiment is a method for transforming a coronavirus S protein or fragment or mutant thereof as defined herein, preferably a trimer of said coronavirus S protein or fragment or mutant thereof as defined herein, from an Apo form to a Holo form comprising the step of contacting the coronavirus S protein or fragment or mutant thereof as defined herein, preferably a trimer as outlined before, with linoleic acid, a derivative, salt or mimetic thereof.

An exemplary embodiment of the transformation method for transforming from Holo to Apo form is generally depicted in FIG. 7 (upper panel) and further outlined below in the description of FIG. 7. Although FIG. 7 mentions the SARS-CoV-2 S protein as a specific example, it is to be understood that the principle shown in FIG. 7 and further described below also refers to the coronavirus S protein or fragment or mutant thereof as defined herein in general. The same applies to “LA” in FIG. 7 which includes LA itself and salts, derivatives as well as mimetics thereof.

For carrying out the methods for producing the complex of the invention as well the transformation method for transforming a coronavirus S protein from an Apo form to a Holo form and the assay methods as disclosed herein, it is preferred that the linoleic acid or derivative or salt or mimetic thereof is present in a composition containing said LA or derivative or salt or mimetic thereof. One preferred example of such a composition is liver cod oil.

Since hydrophobic substances such as fatty acids and related molecules, in particular LA, salts, derivatives and mimetics thereof as disclosed herein show a low degree of water solubility (about 100 mg/l in the case of LA), it is preferred to include an entity into a composition for use in the methods of the invention that increases the solubility of LA (or a derivative, a salt, or a mimetic thereof), in particular in aqueous solutions.

Preferred examples of such entities are proteins that can bind fatty acids, in particular LA (or a derivative or a salt or a mimetic thereof). More preferred proteins of this type are fatty acid binding proteins (FABPs) so that said composition preferably comprises at least one FABP together with LA or a derivative or salt of mutant thereof. FABPs for use in the present invention include FABP1, FABP2, FABP3, FABP4, FABP5, FABP6, FABP7, FABP8, FABP9, FABP10, FABP11 and FABP12. Other preferred solubility improvers are albumin proteins, such as bovine serum albumin (BSA) or human serum albumin (HSA). The composition can therefore comprise one or more of such serum albumin proteins, optionally in addition to at least one FABP.

A further transformation method according to the invention is a method for transforming an isolated coronavirus S protein or fragment or mutant thereof from a Holo form to an Apo form comprising the step of removing a bound linoleic acid, a derivative, salt or mimetic thereof, from the coronavirus spike protein or fragment or mutant thereof, wherein the mutant or fragment of said coronavirus spike protein at least contains a receptor binding domain of said coronavirus spike protein.

Exemplary, and preferred, embodiments of the transformation method for transforming from Apo to Holo form are schematically depicted in FIG. 7 (one embodiment is depicted in the middle panel and a further embodiment is shown in the lower panel) and further outlined below in the description of FIG. 7. Although FIG. 7 mentions the SARS-CoV-2 S protein as a specific example, it is to be understood that the principle shown in FIG. 7 and further described below also refers to the coronavirus S protein or fragment or mutant thereof as defined herein in general. The same applies to “LA” in FIG. 7 which includes LA itself and salts, derivatives as well as mimetics thereof.

The present invention also relates to in vitro assays, i.e. detection methods, in particular detection methods for detecting a) inhibitors of the binding of a complex as disclosed herein to a receptor for the coronavirus S protein or fragment or mutant thereof present in said complex, and/or b) inhibitors of the binding of LA or salts or derivative or mimetics thereof to coronavirus S proteins or fragments or mutants thereof as defined herein.

Thus, in one aspect, the present invention provides an in vitro assay for detecting whether a candidate molecule inhibits the binding of a complex according to the invention to a receptor protein for the coronavirus S protein or fragments or mutant thereof as disclosed herein comprising the steps of:
(a) contacting a complex according to the invention with a receptor protein for the coronavirus S protein as defined herein;
(b) contacting the complex and the receptor of (a) with the candidate molecule; and
(c) detecting unbound receptor and/or unbound coronavirus S protein or mutant or fragment thereof.

In this method, steps (a) and (b) may be performed simultaneously, step (a) may be performed before step (b), or step (b) may be performed before step (a).

According to the invention, it is to be understood that this method, as well as all further detection methods, also detects inhibitors of the interaction, specifically binding, of coronavirus S proteins or fragment or mutants thereof to their receptor.

Preferred embodiments of the above in vitro assay or detection method, respectively, involving steps (a), (b) and (c) are as follows.

A preferred embodiment the above detection method or in vitro assay, respectively, for detecting whether a candidate molecule inhibits the binding of a complex of the invention to the receptor for the coronavirus S protein or fragment or mutant thereof as defined herein is schematically depicted in FIG. 12 and further outlined below in the description of FIG. 12 and Example 11. While FIG. 12 and Example 11 refer to SARS-CoV-2 S protein (or fragments or mutants thereof) as an example, it is to be understood that the principle shown in FIG. 12 and further described in its description and Example 11 below also refer to the coronavirus S protein or fragment or mutant thereof as defined herein in general. The same applies to “LA” in FIG. 12 which includes LA itself and salts, derivatives as well as mimetics thereof.

Further preferred embodiments the above detection method or in vitro assay, respectively, for detecting whether a candidate molecule inhibits the binding of a complex of the invention (and thus, the binding of a coronavirus S protein or fragment or mutant thereof) to the receptor for the coronavirus S protein or fragment or mutant thereof as defined herein are schematically depicted in FIG. 13 and further outlined below in the description of FIG. 13 and Example 12. While FIG. 13 and Example 12 refer to the SARS-CoV-2 S protein (or fragments or mutants thereof) as an example, it is to be understood that the principles shown in FIG. 13 and further described in its description and Example 12 below also refer to the coronavirus S protein or fragment or mutant thereof as defined herein in general. The same applies to “LA” in FIG. 13 which includes LA itself and salts, derivatives as well as mimetics thereof.

Other preferred embodiments of the above in vitro assay or detection method, respectively, for detecting whether a candidate molecule inhibits the binding of a complex of the invention (and thus, the binding of a coronavirus S protein or fragment or mutant thereof) to the receptor for the coronavirus S protein or fragment or mutant thereof as defined herein make use of fragments or mutants, respectively, of the coronavirus S protein, preferably fragments consisting of or comprising a receptor binding domain (RBD) or a mutant thereof. Examples of such fragments and mutants are shown in FIG. 14 and further disclosed in the description of FIG. 14 and Example 13 below. While FIG. 14 and its description and Example 13 refer to SARS-CoV-2 as a specific example, it is to be understood that the same principle as outlined in FIG. 14 and its description and Example 13 can be applied to any of the coronavirus S proteins or fragments or mutants thereof as defined herein.

Such fragments or mutants can be employed in crystallized form as outlined below:

A preferred embodiment of this type of the above in vitro assay or detection method, respectively, for detecting whether a candidate molecule inhibits the binding of a complex of the invention (and thus, the binding of a coronavirus S protein or fragment or mutant thereof) to the receptor for the coronavirus S protein or fragment or mutant thereof as defined herein is schematically depicted in FIG. 15 and further outlined below in the description of FIG. 15 and Example 14. While FIG. 15 and Example 14 refer to the SARS-CoV-2 S protein (or fragments or mutants thereof) as an example, it is to be understood that the principles shown in FIG. 15 and further described in its description and Example 14 below also refer to the coronavirus S protein or fragment or mutant thereof as defined herein in general. The same applies to “LA” in FIG. 15 which includes LA itself and salts, derivatives as well as mimetics thereof.

Further embodiments of the above in vitro assay or detection method, respectively, for detecting whether a candidate molecule inhibits the binding of a complex of the invention (and thus, the binding of a coronavirus S protein or fragment or mutant thereof) to the receptor for the coronavirus S protein or fragment or mutant thereof as defined herein are illustrated in FIG. 16 and further outlined in the description of FIG. 16 as well as in Example 15. It is to be understood that the principle shown according to FIG. 16 as well as described in its description and Example 15 below refer to SARS-CoV-2 coronavirus S protein and fragments or mutants thereof, the same principle can be applied to any of the coronavirus S proteins or fragments or mutants thereof as disclosed herein.

One embodiment of an in vitro assay for detecting whether a candidate molecule inhibits the binding of linoleic acid or derivative or salt or mimetic thereof as disclosed herein to a coronavirus S protein or fragment or mutant thereof as disclosed herein comprises the steps of
i) contacting a complex of the invention with the candidate molecule; and
ii) detecting released linoleic acid or derivative or salt of fragment thereof and/or candidate molecule bound to the coronavirus spike protein or fragment or mutant thereof.

A preferred embodiment of this detection method or in vitro assay, respectively, is schematically depicted in FIG. 8 and further outlined below in the description of FIG. 8 and Example 7. While FIG. 8 and Example 7 refer to SARS-CoV-2 S protein (or fragments or mutants thereof) as an example, it is to be understood that the principle shown in FIG. 8 and further described in its description and Example 7 below also refers to the coronavirus S protein or fragment or mutant thereof as defined herein in general. The same applies to “LA” and “LA*” in FIG. 8 which include LA or labelled LA, respectively, itself and salts, derivatives as well as mimetics thereof.

A further preferred embodiment the above detection method or in vitro assay, respectively, is schematically depicted in FIG. 9 and further outlined below in the description of FIG. 9 and Example 8. While FIG. 9 and Example 8 refer to SARS-CoV-2 S protein (or fragments or mutants thereof) as an example, it is to be understood that the principle shown in FIG. 9 and further described in its description and Example 8 below also refers to the coronavirus S protein or fragment or mutant thereof as defined herein in general. The same applies to “LA” and “LA*” in FIG. 9 which include LA or labelled LA, respectively, itself and salts, derivatives as well as mimetics thereof.

In an embodiment of the above detection method or in vitro assay, respectively, it is possible to first remove LA or a derivative or a salt or a mimetic thereof from a complex according to the invention, and then to carry out the above steps i) and ii). A preferred embodiment of this type is schematically depicted in FIG. 10 and further outlined below in the short description of FIG. 10 and Example 9. While FIG. 10 and Example 9 refer to SARS-CoV-2 S protein (or fragments or mutants thereof) as an example, it is to be understood that the principle shown in FIG. 10 and further described in its short description and Example 9 below also refers to the coronavirus S protein or fragment or mutant thereof as defined herein in general. The same applies to “LA” and “LA*” in FIG. 10 which include LA or labelled LA, respectively, itself and salts, derivatives as well as mimetics thereof.

The present invention provides a further in vitro assay, in particular a competition assay, for detecting whether a candidate molecule inhibits the binding of linoleic acid or derivative or salt or mimetic thereof to a coronavirus S protein or fragment or mutant thereof wherein the fragment or mutant thereof at least contains the receptor binding domain of said coronavirus S protein comprising the steps of:
(A) contacting a coronavirus S protein or fragment or mutant thereof as defined herein with the candidate molecule and linoleic acid or derivative or salt or mimetic thereof; and
(B) measuring the amount of
(B1) the candidate molecule bound to the coronavirus S protein or fragment or mutant thereof as defined herein; and/ or
(B2) measuring the amount of LA or derivative or salt or mimetic unbound to the coronavirus spike protein or fragment or mutant thereof.

A preferred embodiment of this detection method or in vitro assay, respectively, according to the invention is schematically depicted in FIG. 11 and further outlined below in the description of FIG. 11 and Example 10. While FIG. 11 and Example 10 refer to SARS-CoV-2 S protein (or fragments or mutants thereof) as an example, it is to be understood that the principle shown in FIG. 11 and further described in its description and Example 10 below also refers to the coronavirus S protein or fragment or mutant thereof as defined herein in general. The same applies to “LA” in FIG. 11 which includes LA itself and salts, derivatives as well as mimetics thereof.

Preferably, assay methods of the invention comprise the further step of determining a Kd value of the candidate molecule to the coronavirus S protein or fragment or mutant thereof.

Furthermore, detection methods of the invention are preferably carried out with a molar excess of the candidate relative to the concentration of the coronavirus S protein or fragment or mutant thereof.

The inventive assay or detection methods are preferably used for screening multiple different candidates. Such preferred screening methods preferably comprise the steps of:
(I) performing a method as outlined above with multiple different candidate molecules wherein said method is carried out for each candidate molecule in a single reaction;
(II) determining a Kd value for each candidate molecule; and
(Ill) selecting a candidate molecule having a predetermined threshold Kd value to the coronavirus S protein or mutant or fragment thereof.

Preferably, the threshold Kd value is equal or below about 100 nM such as from about 5 to about 99 or about 100 nM , more preferably equal to or below about 10 nM such as from about 1 to about 9 or about 10 nM, even more preferred equal to or below about 5 nM such as from about 1 to about 4,9 or about 5 nM.

Further subject matter of the invention is an in silico method for identifying a compound that interacts with the binding site of a coronavirus S protein for linoleic acid, comprising the steps of:
providing atomic co-ordinates of said binding site of a coronavirus S protein for linoleic acid in a storage medium on a computer; and
using said computer to apply molecular modelling techniques to said co-ordinates.

The in silico method of the invention is preferably carried with the aid of commercially available compound libraries and/or software.

Library Databases: for use in the invention contain theoretical compounds and are available from Enamine, in particular under the following URLs (as of 18 Jun. 2020):
https://enamine.net/library-synthesis/real-compounds/real-database
https://enamine.net/library-synthesis/real-compounds/real-compound-libraries

Physical compound libraries for use in the invention include fragments and full drug like compounds and are also commercially available from Enamine:
For fragments, it is referred to Enamine e.g. https://enamine.net/fragments

For screening libraries is referred to, e.g.,
https://enamine.net/hit-finding/compound-collections/screening-collection.

In silico software for use in the present invention is commercially available, e.g. from Schrodinger, New York, N.Y., USA, and Biovia, San Diego, Calif., USA. Is referred to the following URLs:
https://www.schrodinger.com/glide
https://www.schrodinger.com/qm-polarized-ligand-docking
https://www.schrodinger.com/desmond
https://www.3dsbiovia.com/products/collaborative-science/biovia-discovery-studio/

All URLs referred to herein are understood as of 18 Jun. 2020.

Typically, the in silico method results in one or more, typically in a multitude, of candidate molecules which are then further validated with the above-described assay methods of the invention.

Linoleic acid (LA), or salts or derivatives or mimetics thereof, as described herein, can be used for treatment and/or prevention of coronavirus infections, wherein the derivative, salt of mimetic binds to the coronavirus S protein of said coronavirus. The present invention is also directed to methods for prevention and/or treatment of a coronavirus infection comprising the step of administering an effective amount of LA or a salt or a derivative or mimetic thereof to a subject, preferably a human subject, in need thereof, wherein the derivative, salt of mimetic binds to the coronavirus S protein of said coronavirus. The present invention is also directed to the use of LA, or salts or derivatives or mimetics thereof, as described herein, for the preparation of a medicament for the treatment and/or prevention of a coronavirus infection.

In the context of the invention “treatment” means that the condition of the subject, preferably a human, is at least meant to result in a prevention of further progression such as, in particular in case of SARS or MERS infections, most preferably SARS-Cov-2 infections, that the state of a patient suffering from early symptoms such as cough and/or loss of taste and/or loss of ability to smell and/or mild fever of not more than 38° C., more preferably not more than 38.5° C., and the like does not progress to a state where the patient suffers from higher fever and/or respiratory distress. In preferred embodiments of the invention, the treatment results in a state where the patient is incapable of further transmitting the coronavirus, preferably a SARS-CoV-2 virus to other subjects. Such a non-transmissible state is typically reached when a coronavirus PCR test, preferably a SARS-CoV-2 PCR test, of material taken from the nasal and/or throat mucosa of the patient shows no detectable threshold signal after at least about 33, 34 or 35 PCR cycles.

The same conditions apply to the term “prevention” as used in the present invention. That is, prevention typically relates to a prophylactic treatment of a subject, preferably a human subject. Preferably, prevention means that the subject does not show any signs of a coronavirus infection, preferably a SARS or MERS, more preferably a SARS-CoV-2 infection, preferably no signs of cough and/or loss of taste and/or loss of ability to smell. In other embodiments, prevention means that a patient over the treatment period and preferably at least about 1 week to about 8 weeks thereafter shown no coronavirus infection as evidenced by a negative coronavirus PCR test, preferably a SARS-CoV-2 PCR test In certain other preferred embodiments, the prophylactic treatment involves the prevention of transmitting a coronavirus infection, preferably a SARS or MERS, more preferably a SARS-CoV-2 infection, by a treated patient as evidenced by a coronavirus PCR test, preferably a SARS-CoV-2 PCR test, of material taken from the nasal and/or throat mucosa of the patient showing no detectable threshold signal after at least about 33, 34 or 35 PCR cycles.

In preferred embodiments of the invention concerning methods of prevention and/or treatment of coronavirus infections as well as corresponding uses, the linoleic acid or derivative or salt of mimetic thereof is preferably used in a non-vesicular form. According to the present invention the term “present in non-vesicular form” means that, contrary to prior art approaches, the linoleic acid or derivative or salt of mimetic thereof does not form vesicular structures such as liposomes in the respective use or method, respectively. Typically, the linoleic acid or derivative or salt of mimetic thereof, is used in a composition, and it is not present in vesicles, be they paucilamellar or multilamellar, the composition is monophasic, with aqueous monophasic compositions or solutions being particularly preferred. Aqueous compositions according to the invention or for use in the invention typically comprise water or an aqueous electrolyte solution or buffer such as a physiological NaCI solution, phosphate buffered saline (PBS), Ringer or Ringer Lactate. In other preferred embodiments of the invention the non-vesicular lipid (i.e. linoleic acid or derivative or salt of mimetic thereof as described herein) composition is in the form of a dry powder. In preferred powder embodiments of the invention, the powder is prepared by drying, such as freeze-drying, a liquid composition comprising the linoleic acid or derivative or salt of mimetic thereof as described herein in non-vesicular form. Most preferred, the dry powder is prepared by drying such as freeze-drying a monophasic solution, more preferably a monophasic aqueous solution of the linoleic acid or derivative or salt of mimetic thereof as described herein. If necessary for the desired application, an initially prepared dried product can be ground to an appropriate powder (average) particle size. Preferably, compositions of the invention or for use in the invention are sterilized.

The preparation of non-vesicular compositions according to the invention typically comprises the step of adding a desired amount of the linoleic acid or derivative or salt or mimetic thereof to a, preferably stirred, composition, preferably a solution, of a solubilizer (hereinafter also denoted as “fatty acid solubilizer”), preferably a solubilizer or mixture of solubilizers as further outlined below, most preferred one or more cyclodextrins, optionally containing further ingredients as further outlined below, whereby the linoleic acid or derivative or salt or mimetic thereof may itself be present, potentially in vesicular form, in a pre-prepared composition containing it. If used directly, the linoleic acid or derivative or salt or mimetic thereof is pre-heated before adding to the solubilizer composition to a temperature at or above the melting point of the linoleic acid or derivative or salt or mimetic thereof. After addition of the linoleic acid or derivative or salt or mimetic thereof to the solubilizer, the combined are sonicated and/or stirred for an appropriate amount of time such as about 30 min to about 48 hours. Preferred are combinations of sonification, for example for about 15 min to about 2 hours, preferably for about 30 min to about 1 hour, followed by stirring for about 1 hour to about 48 hours, preferably about 1 hours to about 24 hours.

An “effective amount” of an active substance for use in the inventive therapeutic or preventive, respectively, uses and methods as disclosed herein, preferably LA or a salt or a derivate of mimetic thereof as defined herein or of an inhibitor as disclosed herein is an amount of the active substance(s) exerting an effect suitable for at least improving the condition, in particular of a coronavirus infection as disclosed herein, especially COVID-19, preferably substantially improving said condition, optimally curing said condition.

With respect to LA or a salt or a derivate of mimetic thereof as defined herein, most preferred LA, an effective amount is preferably in the range in a daily dose, which may be administered in one or more unit doses, of about 1 mg/kg body weight to about 1400 mg/kg body weight, more preferably about 50 mg/kg body weight to about 500 mg/kg body weight, wherein body weight means weight of the subject treated.

With respect to the inhibitors of the invention, a preferred effective amount is, e.g., a daily dose, which may be administered in one or more unit doses, of about 1 mg/kg bodyweight to about 100 mg/kg bodyweight, more of about 1 mg/kg bodyweight to about 10 mg/kg bodyweight, wherein body weight means weight of the subject treated.

Furthermore, the above detection method provide inhibitors of the interaction, preferably binding, of a coronavirus S protein to its receptor, which means that the inhibitor interferes with the interaction of the coronavirus S protein with its receptor. Preferably, the inhibitor is a candidate molecule selected according to a Kd value as outlined above.

Inhibitors as defined herein are useful for the treatment and/or prevention of a coronavirus infection. The present invention is also directed to methods for prevention and/or treatment of a coronavirus infection comprising the step of administering an effective amount at least one inhibitor as defined herein to a subject, preferably a human subject, in need thereof. The present invention is also directed to the use of at least one inhibitor for the preparation of a medicament for the treatment and/or prevention of a coronavirus infection.

It is also contemplated that LA (or a salt or a derivative or a mimetic thereof) is combined with one or more inhibitors in the therapeutic and/or preventive uses and methods as disclosed herein.

Preferably, the coronavirus infection is an infection by a coronavirus causing a respiratory disease, in particular pneumonia, preferably in humans. More preferred, the coronavirus infection is an infection by SARS-CoV, MERS-CoV and/or SARS-CoV-2, with infections by SARS-CoV-2 are most preferred.

The LA or derivative or salt or mimetic thereof and/or the at least one inhibitor may be administered systemically and/or topically to a subject.

With respect to “topical administration” it is to be understood that, according to the invention, this term is considered to be the route of administration, and shall include administrations occurring locally, but having a systemic pharmacological effect.

A preferred topical administration of the active substance(s) (i.e.LA or derivative or salt or mimetic thereof and/or an inhibitor as defined herein) involves administration to the respiratory tract of subject, including administration to the upper respiratory tract such as nasal and/or pharyngeal and/or laryngeal administration and/or administration to the mouth, and/or lower respiratory tract such as administration to the trachea and/or primary bronchi and/or lungs of a subject, preferably as an aerosol formulation, more preferably as a spray formulation, of the active compound(s).

In preferred embodiments of the invention, the one or more active substance(s) (i.e.LA or derivative or salt or mimetic thereof and/or an inhibitor as defined herein), more preferably as an aerosol formulation, are administrated to the upper respiratory tract, more preferably by nasal administration. Nasal administration, also synonymously referred to according to the invention as “intra-nasal” administration, generally includes administration to the nasal cavity, and preferably comprises administration into one or both, preferably both, nostrils.

Systemic administration of the active substance(s) (i.e. LA acid or derivative or salt or mimetic thereof and/or the inhibitor as defined herein) is preferably carried out by administering the active substance(s) orally and/or by intra-venous infection to a subject.

Oral administration can by embodied by mixing the active substance(s) with a food.

Preferably, the LA or derivative or salt or mimetic thereof, or the inhibitor(s) is/are administered in the form of a composition comprising the LA or derivative or salt or mimetic thereof and/or the at least one inhibitor in combination with at least one, optionally pharmaceutically acceptable, carrier for lipophilic substances.

According to the invention, carriers for lipophilic substances can be components having one or more binding sites for LA or derivative or salt or mimetic thereof as defined herein and/or for the inhibitor. According to the invention a carrier for LA or derivative or salt or mimetic thereof as defined herein and/or for the inhibitor can also be a solubilizer for lipophilic substances such as LA or derivative or salt or mimetic thereof as defined herein and/or for the inhibitor.

Preferred suitable carriers for lipophilic substances include, but are not limited to, proteins, lipoproteins, synthetic nanoparticles, carbohydrate matrices etc., preferably proteins, lipoproteins, synthetic nanoparticles, carbohydrate matrices etc. having one or more binding sites for LA or derivative or salt or mimetic thereof as defined herein and/or for the inhibitor. Suitable carriers for use in the invention are disclosed, e.g. in Kalepou et al. (2013) Acta Pharmaceutica Sinica B 3 (6), 361-372.

Preferred proteins of this type are, e.g., albumin proteins, preferably human serum albumin, and fatty acid binding proteins, preferably one or more selected from FABP1, FABP2, FABP3, FABP4, FABP5, FABP6, FABP7, FABP8, FABP9, FABP10, FABP11 and FABP12.

In the therapeutic and/or preventive methods, uses and compositions as disclosed herein, it is particularly preferred when the LA or derivative or salt or mimetic thereof as defined herein and/or the at least one inhibitor as defined herein is mixed with the carrier, preferably a carrier having one or binding sites for lipophilic substances and/or a solubilizer for lipophilic substances, ex vivo before administration to the subject.

In addition to the above components for use in preventive and/or therapeutic methods and uses as disclosed herein, compositions for use in the invention containing the above active components, preferably in admixture with a lipophilic carrier, preferably a carrier having one or binding sites for lipophilic substances and/or a solubilzer for lipophilic substances, may contain further ingredients typically present in dosage forms for topical application such as administration to the respiratory tract, preferably nasal administration, or systemic application such as oral or intra-venus application for providing and/or improving various parameters. Typical additional ingredients for use in the present invention include excipients, other carriers, fillers, glidants, dispersants, plasticizers, wetting agents, anti-tacking agents, neutralization agents, colorants, pigments, opacifiers, flavours, taste improvement agents such as sweeteners, buffers, injection-aids, and the like. The person skilled in the art of formulating compositions for preventive and/or therapeutic uses and methods as disclosed herein is readily able to identify specific compounds and substances of the above and other types as well as their combinations and amounts to be used. Further guidance can be found in Remington's Pharmaceutical Sciences, 18th Ed. Mack Printing Company, 1990, in particular pages 1289-1329.

Preferred compositions in the context of the invention, in particular for administration of LA or derivative or salt or mimetic thereof as defined and described herein to the respiratory tract, more preferably to the upper respiratory tract, most preferred for nasal administration, contain at least one solubilizer for said LA or derivative or salt or mimetic thereof as defined and described herein, more preferably a compound of formula (I), most preferred LA and/or oleic acid.

Preferred solubilizers for lipophilic substances in the context of the invention are cyclodextrin, ethanol, propylene glycol and a polypropylene glycol, including mixtures of two or more us such solubilizers.

The solubilizer(s) is/are and their amounts, in particular their molecular ratio to the LA or derivative or salt or mimetic thereof as defined and described herein, more preferably a compound of formula (I), most preferred LA and/or oleic acid, are preferably selected such that they substantially prevent formation of lipid vesicles, which will regularly form in particular in aqueous compositions. Preferred solubilizers prevent hydrophobic interaction between individual active substances (i.e. LA or derivative or salt or mimetic thereof as defined and described herein, more preferably a compound of formula (I), most preferred LA and/or oleic acid) by complexing the lipid molecules in a form such that they are shielded from a polar environment, in particular in aqueous solutions.

An especially preferred solubilizer for use in the invention is cyclodextrin (CD). Preferably, the cyclodextrin is an □-cyclodextrin, a β-cyclodextrin, a γ-cyclodextrin, a □-cyclodextrin or a mixture of two or more thereof.

In other preferred embodiments, the cyclodextrin, preferably an □-cyclodextrin, a β-cyclodextrin, a γ-cyclodextrin, a □-cyclodextrin or a mixture of two or more thereof, is a cyclodextrin derivative selected from O-methylated, acetylated, hydroxypropylated, hydroxyethylated, hydroxyisobutylated, glucosylated, maltosylated and sulfoalkylether cyclodextrin and mixtures of two or more thereof. Derivatives of cyclodextrins are disclosed, e.g. in U.S. Pat. No. 5,760,017 and WO 91/13100 A1 Most preferred, the cyclodextrin is a β-cyclodextrin and/or a derivative thereof, preferably hydroxypropyl-β-cyclodextrin, such as 2 hydroxypropyl-β-cyclodextrin and/or 3-hydroxypropyl-β-cyclodextrin and/or 2,3-dihydroxypropyl-β-cyclodextrin, dimethyl-β-cyclodextrin, trimethyl-β-cyclodextrin, randomly methylated β-cyclodextrin, hydroxyethyl-β-cyclodextrin, 2-hydroxyisobutyl-β-cyclodextrin, glucosyl-β-cyclodextrin and maltosyl-β-cyclodextrin. Highly preferred cyclodextrins for use in the present invention are sulfoalkylether-modified cyclodextrins, preferably mixtures of such sulfoalkylether cyclodextrins, more preferred sulfoalkylether β-cyclodextrins, most preferred sulfobutylether-ss-cyclodextrin. Preferred alkylated and sulfoalkylated cyclodextrins and mixtures of such alkylated and sulfoalkylether dextrins are described in WO 98/50077 A1, WO 00/41704 A1, WO 2007/050075 A1, WO 2009/013434 A2, WP 2009/018069 A2, WO 2013/13666 A1, WO 2016/029179 A1 and WO 2021/101842 A1. Preferred sulfoalkylether cyclodextrins, in particular mixtures of such sulfalkylether cyclodextrins, for use in the invention are available under the trademark Captisol®.

In preferred embodiments of the invention, the molar ratio of cyclodextrin, preferably a cyclodextrin selected from one or more of the above preferred embodiments, to LA or a derivative or salt or mimetic thereof, most preferred LA and/or OA, in the composition of the invention (including uses of the compositions and methods of treatments and/or prevention), preferably in the form of a monophasic composition, most preferred a monophasic aqueous composition, is at least about 5:1, about 6:1, about 7:1, about 8:1, about 9:1, or about 10:1, more preferred from about 10:1 to about 60:1, still more preferred about 10:1 to about 50:1, even more preferred about 10:1 to about 40:1, most preferred about 10:1 to about 30:1. Preferred compositions of this type comprise about 0.5 mM to about 3 mM lipid and about 5 mM or 10 mM to about 30 mM cyclodextrin.

In preferred embodiments of the invention, a unit dose of a composition for nasal administration contains about 1 to 84 μg, more preferably about 20 μg, linoleic acid or a derivative or a salt or a mimetic thereof as described herein, most preferred LA and/or OA, in non-vesicular form, preferably in a unit dose volume of about 25 to about 300 μI of preferably aqueous composition, more preferably aqueous solution as further elaborated herein. Nasal administration involves preferably the application of a unit dose, more preferably a unit dose as outlined before, one to about five times per day, more preferably three times per day.

In preferred embodiments of the invention, a unit dose of a composition for administration to the lower respiratory tract, preferably for pulmonary administration, contains about 1 to 500 μg, more preferably about 20 μg, linoleic acid or a derivative or a salt or a mimetic thereof as described herein, most preferred LA and/or OA, in non-vesicular form. In case of administration as a liquid, preferably an aqueous composition, more preferably in an aqueous solution as further elaborated herein, the unit dose is preferably in a volume of about 25 to about 300 μI. In other forms of administration to the lower respiratory tract, the above preferred unit dose is in the form of a dry powder composition. Administration to the lower respiratory tract, preferably pulmonary administration, involves preferably the application of a unit dose, more preferably a unit dose as outlined before, one to about five times per day, more preferably three times per day.

In certain embodiments of the invention, especially in the context of administration to the upper respiratory tract, preferably nasal administration, and/or lower respiratory tract, preferably pulmonary administration, oleic acid is most preferably used as the API.

In certain preferred embodiments of the invention the solubilizer is ethanol, or at least one of the solubilizers is ethanol.

In certain embodiments, the composition of the invention, in particular for administration of LA or derivative or salt or mimetic thereof as defined and described herein to the respiratory tract, more preferably to the upper respiratory tract, most preferred for nasal administration, contain, preferably in addition to the at least one carrier, more preferably the at least one solubilizer, at least one antioxidant.

Preferred examples of useful antioxidants in the context of the invention include, but are not limited to, ascorbic acid, tocopherols, EDTA, butylated hydroxytoluene (BHT), butylated hydroxyanisole (BHA), propyl gallate, ascorbyl fatty acid esters and mixtures of two or more thereof.

In further preferred embodiments, the composition of the invention, in particular for administration of LA or derivative or salt or mimetic thereof as defined and described herein to the respiratory tract, more preferably to the upper respiratory tract, most preferred for nasal administration, contain, preferably in addition to the at least one carrier, more preferably the at least one solubilizer, further contains at least one mucoadhesive.

Preferred examples of useful mucoadhesives in the context of the invention include, but are not limited to, cellulose and derivatives thereof, more preferably methylcellulose, hydroxypropyl methylcellulose (also referred to as “Hypromellose”), microcrystalline cellulose, carboxymethylcellulose, hydroxyethyl cellulose or a mixture of two or more thereof.

In further preferred embodiments, the composition of the invention, in particular for administration of LA or derivative or salt or mimetic thereof as defined and described herein to the respiratory tract, more preferably to the upper respiratory tract, most preferred for nasal administration, contain, preferably in addition to the at least one carrier, more preferably the at least one solubilizer, and optionally at least one mucoadhesive, contains at least one propellant.

A preferred example of useful propellants in the context of the invention is a hydrofluoroalkane, more preferably HFA 227, HFA 134a or a mixture thereof.

Preferred embodiments of compositions of the invention, in particular for administration of LA or derivative or salt or mimetic thereof as defined and described herein to the respiratory tract, more preferably to the upper respiratory tract, most preferred for nasal administration, contain said LA or derivative or salt or mimetic thereof as defined and described herein, more preferably a compound of formula (I), most preferred LA and/or oleic acid and:
a cyclodextrin
EDTA
a hypromellose

Further preferred embodiments of compositions of the invention, in particular for administration of LA or derivative or salt or mimetic thereof as defined and described herein to the respiratory tract, more preferably to the upper respiratory tract, most preferred for nasal administration, contain said LA or derivative or salt or mimetic thereof as defined and described herein, more preferably a compound of formula (I), most preferred LA and/or oleic acid and:

propylene glycol or a polypropylene glycol
EDTA
BHA and/or BHT
a Hypromellose; and, optionally
a cyclodextrin.

Other preferred embodiments of compositions of the invention, in particular for administration of LA or derivative or salt or mimetic thereof as defined and described herein to the respiratory tract, more preferably to the upper respiratory tract, most preferred for nasal administration, contain said LA or derivative or salt or mimetic thereof as defined and described herein, more preferably a compound of formula (I), most preferred LA and/or oleic acid and:
propylene glycol or a polypropylene glycol
BHT and/or BHA
a propellant

Still other preferred embodiments of compositions of the invention, in particular for administration of LA or derivative or salt or mimetic thereof as defined and described herein to the respiratory tract, more preferably to the upper respiratory tract, most preferred for nasal administration, contain said LA or derivative or salt or mimetic thereof as defined and described herein, more preferably a compound of formula (I), most preferred LA and/or oleic acid and:
a cyclodextrin
ethanol
EDTA
ascorbic acid.

Yet other preferred embodiments of compositions of the invention, in particular for administration of LA or derivative or salt or mimetic thereof as defined and described herein to the respiratory tract, more preferably to the upper respiratory tract, most preferred for nasal administration, contain said LA or derivative or salt or mimetic thereof as defined and described herein, more preferably a compound of formula (I), most preferred LA and/or oleic acid, and at least one oxidant, and:
ethanol
a propellant; and, optionally
a tocopherol.

Further subject matter of the invention is a pharmaceutical delivery device, preferably a respiratory delivery device, containing a composition of the invention, in particular for administration of LA or derivative or salt or mimetic thereof as defined and described herein to the respiratory tract, more preferably a delivery device adapted for intra-nasal and/or pulmonary delivery of said composition. The pharmaceutical delivery device is preferably a nebulizer or a vaporizer, which may preferably take the form of an inhaler or a pump spray device. Preferred examples of delivery devices, in particular devices for respiratory delivery, include, but are not limited to, a nebulizer, vaporizer, vapor inhaler, squeeze bottle, metered-dose spray pump, Bi-dir Multi-dose spray pump , a gas driven spray system/atomizer, electrically powered Nebulizers/Atomizers, mechanical powder sprayers, breath actuated inhaler, insufflator, meter dose inhaler and a dry powder inhaler. An overview of preferred examples of respiratory delivery devices of the invention or for use in the invention can be found, e.g. in F.G. Djupesland (2013) Drug Deliv. and Transl. Res. 3, pages 42-62.

The device of the invention is particularly useful in the prevention and/or treatment of a viral infection, preferably a viral respiratory infection, more preferably a viral respiratory infection caused by a coronavirus, most preferably a viral infection caused by SARS-CoV and/or MERS and/or SARS-CoV-2, the latter being the especially preferred target for use of the pharmaceutical device of the invention.

Further subject matter of the invention is a method for selecting binder molecules binding to the complex according to the invention from a library of multiple candidate binder molecules comprising the steps of:
(α) contacting the complex of the invention with the library of multiple candidate binder molecules; and
(β) detecting which of the multiple candidate binder molecules have bound to the complex.

Preferred binder molecules in the context of the above selection method are antibodies, antibody fragments, antibody mimetics, peptides, aptamers, darpins, DNAs, RNAs, peptide nucleic acids, heterocycles and glycoconjugates.

It is evident to the skilled person that the selection method of the invention identifying binder molecule being selected from antibodies, antibody fragments, antibody mimetics, peptides, aptamers and darpins makes use of the complex of the invention as an antigen.

Preferred antibody fragments or antibody mimetic for use in the selection method are selected from, but not limited to, scFv, sulfide-bond stabilized scFv (ds-scFv), Fab, single domain antibody (sdAb), VHH/VH of camelid heavy chain antibody, single chain Fab (scFab), di- or multimeric antibody format including dia-, tria- and tetra-body and a minibody (miniAb) that comprise different formats consisting of scFvs linked to oligomerization domains.

The term “antibody fragment” generally refers to a part of an antibody which retains the ability of the complete antibody to specifically bind to an antigen. As already mentioned above, examples of antibody fragments include, but are not limited to, Fab fragments, Fab' fragments, F(ab′)2 fragments, heavy chain antibodies, single-domain antibodies (sdAb), scFv fragments, fragment variables (Fv), VH domains, VL domains, nanobodies, IgNARs (immunoglobulin new antigen receptors), di-scFv, bispecific T-cell engagers (BITEs), dual affinity re-targeting (DART) molecules, triple bodies, diabodies, a single-chain diabody and the like.

A “diabody” is a fusion protein or a bivalent antibody which can bind different antigens. A diabody is composed of two single protein chains (typically two scFv fragments) each comprising variable fragments of an antibody. Diabodies therefore comprise two antigen-binding sites and can, thus, target the same (monospecific diabody) or different antigens (bispecific diabody).

The term “single domain antibody” as used in the context of the present invention refers to antibody fragments consisting of a single, monomeric variable domain of an antibody. Simply, they only comprise the monomeric heavy chain variable regions of heavy chain antibodies produced by camelids or cartilaginous fish. Due to their different origins they are also referred to VHH or VNAR (variable new antigen receptor)-fragments. Alternatively, single-domain antibodies can be obtained by monomerization of variable domains of conventional mouse or human antibodies by the use of genetic engineering. They show a molecular mass of approximately 12-15 kDa and thus, are the smallest antibody fragments capable of antigen recognition. Further examples include nanobodies or nanoantibodies.

Antigen-binding entities useful in the context of the invention also include “antibody mimetic” which expression as used herein refers to compounds which specifically bind antigens similar to an antibody, but which compounds are structurally unrelated to antibodies. Usually, antibody mimetics are artificial peptides or proteins with a molar mass of about 3 to 20 kDa which comprise one, two or more exposed domains specifically binding to an antigen. Examples include inter alia the LACI-D1 (lipoprotein-associated coagulation inhibitor); affilins, e.g. human-y B crystalline or human ubiquitin; cystatin; Sac7D from Sulfolobus acidocaldarius; lipocalin and anticalins derived from lipocalins; DARPins (designed ankyrin repeat domains); SH3 domain of Fyn; Kunits domain of protease inhibitors; monobodies, e.g. the 10thtype III domain of fibronectin; adnectins: knottins (cysteine knot miniproteins); atrimers; evibodies, e.g. CTLA4-based binders, affibodies, e.g. three-helix bundle from Z-domain of protein A from Staphylococcus aureus; Trans-bodies, e.g. human transferrin; tetranectins, e.g. monomeric or trimeric human C-type lectin domain; microbodies, e.g. trypsin-inhibitor-II; affilins; armadillo repeat proteins. Nucleic acids and small molecules are sometimes considered antibody mimetics as well (aptamers), but not artificial antibodies, antibody fragments and fusion proteins composed from these. Common advantages over antibodies are better solubility, tissue penetration, stability towards heat and enzymes, and comparatively low production costs.

In preferred embodiments, the selection method uses a library of nucleic acids encoding said antibodies, antibody fragments, antibody mimetics, peptides or darpins.

Especially in the context of the selection method of the invention, preferably using the complexes of the invention as antigens for the selection method, but also with respect to the detection methods for identifying candidate inhibitors as disclosed herein, preferably those interfering in the interaction between a coronavirus S protein and its receptor as disclosed above, it is possible to include a selection and/or evolutionary process for providing target binding-optimized sequences such as optimized antigens to exert an improved immune response thereto. One preferred process is ribosome display as outlined in detail in Schaffitzel et al. (2001) in: Protein-Protein Interactions, A Molecular Cloning Manual: In vitro selection and evolution of protein-ligand interaction by ribosome display (Golemis E., ed.), pages 535-567, Cold Spring Harbor Laboratory Press, New York. The ribosome display protocol has the advantage of being carried out completely in vitro at all steps of the selection process. Further preferred selection processes are also known in the art and include phage display (Smith (1985) Science 228, 1315-1317; Winter et al. (1994) Annu. Rev. Immunol. 12, 433-455), yeast two-hybrid systems (Fields and Song (19899 Nature 340, 245-246; Chien et al. (1983) Proc. Natl. Acad. Sci. U.S.A. 88, 9578-9582), and cell surface display methods (Georgiu et al. (1993) Trends Biotechnol. 11, 6-10; Boder and Wittrup (1997) Nat. Biotechnol. 15, 553-557). Further selection process includes mRNA display, baculo display, yeast display and other cell surface display technologies.

An exemplary selection method according to the invention based on phage display is depicted in FIG. 17, and further detailed in its description and Example 16 outlined below. It is to understood that, while FIG. 17, its below description and Example 16 refer to SARS-CoV-2 and LA, the exemplary embodiment can as well applied to any of the coronavirus S protein complexes as disclosed and defined herein.

As a further example, the ribosome display process can basically be used in two ways for selection and optimization of binder molecules for the complexes of the invention. Either, a potential binder molecule sequence can be selected first from an initial library of polypeptides sequences that can be as large as 1014 individual sequences, more typically 109 to 1010 sequences, optionally employing evolutionary procedures as described in detail in Schaffitzel et al. (2001), supra. After selection of the optimized antigen-binding sequences, i.e. complex-binding sequences, the nucleotide sequence encoding it is cloned into an appropriate vector such that a polypeptide is expressed.

According to an alternative embodiment of this aspect of the invention, a library of potential antigen-binding (i.e. complex-binding) polypeptide encoding sequences is directly cloned into a nucleic acid such that each sequence encodes a polypeptide potentially binding to a complex of the invention. The polypeptides comprising an initial library of antigen-binding sequences are than expressed in vitro and selection of optimized antigen-binding sequences is carried out according to the ribosome display methodology as outlined in detail in Schaffitzel et al. (2001), supra.

The complexes of the invention can further be used for the production of antibodies binding a complex of the invention, preferably wherein a non-human animal is immunized with a complex of the invention.

A subject matter of the invention making use of the complexes of the invention for the production of antibodies as described above is a method for producing antibodies binding to a complex according to the invention comprising the steps of
immunizing a non-human animal with said complex; and
isolating antibodies binding to said complex form said animal.

Preferably the (non-human) animal is a mammal or a cartilaginous fish. Preferred mammals for use the production of antibodies as disclosed herein are, e.g., primates, rodents, dromedaries, camels, llamas and alpacas. Preferred cartilaginous fishes for use in antibody production as disclosed herein are sharks.

Binder molecules selected by the selection method according to the invention as well as antibodies produced by the method for antibody production according to the invention can be used in the treatment and/or prevention of a coronavirus infection wherein the binder molecule or antibody, respectively, binds to the coronavirus spike protein of said coronavirus. The invention also relates to corresponding treatment methods comprising the step of administering to a subject in need thereof, in particular a subject having coronavirus infection, an effective amount of the binder molecule and/or the antibody selected or produced, respectively, according to the invention.

Binder molecules selected by the selection method according to the invention and/or antibodies produced according to the invention can preferably be used in in vitro diagnostic systems for detection of coronavirus infection, wherein the selected binder molecule or the produced antibody, respectively, binds to the coronavirus spike protein of said coronavirus.

Furthermore, the invention discloses the use of the complex according to the invention in in vitro diagnostic systems for detection of coronavirus infection.

### BRIEF DESCRIPTION OF THE DRAWINGS

The Figures show:

FIG. 1A: LA-bound SARS-COV-2 S protein interaction with ACE2. In FIG. 1A the complex LA-bound SARSV-COV-2 S protein with ACE2 was analyzed by size exclusion chromatography (SEC) evidencing formation of a complex between the purified proteins. FIG. 1A Left: SEC profiles are shown with peak fractions labelled for ACE2 (III.), LA-bound Spike (II.) and a 1:1 mixture (I.) are shown. FIG. 1A Right: the peak fractions I., II. and III. were analyzed by denaturing polyacrylamide gel electrophoresis (SDS-PAGE) stained with Coomassie Brilliant Blue. Sections of the corresponding SDS-PAGE gels are shown. Molecular weights of a protein standard are given with numbers representing kilodaltons (kDa). Bands in the SDS-PAGE gel sections corresponding to Spike and ACE2 proteins are marked. FIG. 1B Chemical structure and calculated molecular weight of LA are indicated. FIG. 1C LC-MS analysis of highly purified S protein sample. C4 column elution profile (middle) and ESI-TOF spectra of the C4 peak elution fraction (below) are shown. The molecular weight corresponding to the peak in ESI-TOF is indicated.

FIG. 2: Cryo-EM image processing workflow of LA-bound SARS-COV-2 S protein. A motion-corrected cryo-EM micrograph is shown (scale bar 20 nm), reference-free 2D class averages (scale bar 10 nm), 3D classification and refinement resulting in cryo-EM maps corresponding to the open conformation and the closed conformation (not symmetrized and C3 symmetrized).

FIG. 3: Ribbon Diagram showing the receptor binding domain (RBD) of the SARS-CoV-2 S protein containing bound linoleic acid (LA), interacting with its target receptor for cell entry, angiotensin-converting enzyme 2 (ACE2). The part of the Spike RBD directly interacting with ACE2, the so-called Achilles Heel, is colored in dark grey and labelled (AH). The RBD and ACE2 (colored in grey) are shown in a cartoon representation; the LA is shown as spheres.

FIG. 4: Structure of SARS-CoV-2 S protein LA binding pocket seen in a close-up view. Portions of the two adjacent RBDs from LA-bound SARS-CoV-2 Spike are shown (colored in light grey), labelled RBD1 and RBD2. LA is shown in a stick representation. RBD1 is superimposed on the ‘apo’ form of the RBD (colored in dark grey) comprising no LA. The lateral movement of the gating helix (GH) to avoid a steric clash with LA is evident.

FIG. 5: The architecture of the three RBDs in the structure of ligand-free ‘apo’ SARS-CoV-2 S protein. A schematic drawing showing ‘apo’ SARS-CoV-2 S protein containing no LA (colored in light grey) is shown, superimposed on the structure of LA-bound SARS-CoV-2 S protein (colored in dark grey) looking down the Spike. LA in the RBD on the bottom is shown as spheres (omitted for better clarity in the other two RBDs from LA-bound Spike). Arrows indicate molecular movements accompanying the conformational switch resulting in a condensed RBD trimer in the LA-bound (‘holo’) form. The three RBDs forming the trimer in the apo form are denoted RBD1 apo, RBD2 apo and RBD3 apo, respectively. The three RBDs in the LA-bound form are denoted RBD1, RBD2 and RBD3, respectively. RBD and RBD1 apo, shown at the bottom, were used to align the apo and LA-bound forms of the SARS-CoV-2 S protein.

FIG. 6: In vitro enzyme-linked immunosorbent assay (ELISA) evidencing LA-bound SARS-CoV-2 S protein binding to immobilized ACE2 receptor is shown. The absorption at 450nm (normalized units) is plotted against the concentrations in a serial dilution of the S protein in phosphate buffered saline (PBS). Error bars indicate standard deviations from three independent replicates. The S protein competes with a purified RBD horse-radish peroxidase (HRP) fusion protein for ACE2 binding (see inset).

FIG. 7: Methods for production of Holo and Apo forms of SARS-CoV-2 S protein. Shown are schematic diagrams describing methods to produce SARS-CoV-2 S protein containing bound LA (Holo) and SARS-CoV-2 S protein without bound LA (Apo). Top: indicated schematically, SARS-CoV-2 S protein is produced in an expression system with supplemental LA provided, e.g. via cod liver oil, which results in the Holo form of the SARS-CoV-2 S protein. Indicated by the black rectangle is the LA binding pocket occupied by LA. Center: indicated schematically, SARS-CoV-2 S protein is produced in an expression system with supplemental LA provided which results in the Holo form of the SARS-CoV-2 S protein. Indicated by the black rectangle is the LA binding pocket. Stripping of bound LA can be carried out via e.g. detergent washing, to produce the Apo form of the SARS-CoV-2 S protein. Detergent stripping could be carried out on size exclusion column to permanently eliminate LA from the protein sample. Bottom: indicated schematically, SARS-CoV-2 S protein is produced in an expression system without supplemental LA provided, e.g. a mammalian transient transfection expression system with serum free media (24) to produce the Apo form of the SARS-CoV-2 S protein.

FIG. 8: Example 7. Screening method for discovery, i.e. identification, of inhibitors of LA binding to SARS-CoV-2 S protein. Shown is a schematic diagram describing a high throughput-compatible process to discover potential small molecule or biologic drug candidates which are inhibitors of LA binding to SARS-CoV-2 S protein. Left: indicated schematically is SARS-CoV-2 S protein, immobilized on to the surface of a microplate in aqueous solution optionally containing a carrier protein for LA such as BSA or a Fatty Acid Binding Protein (FABP). Indicated by the black rectangle is the LA binding pocket, which is occupied by LA. Center Black Arrow: a molar excess of a small molecule or biologic drug candidate is added to the solution of immobilized SARS-CoV-2 S protein with bound LA. Right: LA is displaced and replaced by drug candidate in the LA binding pocket. LA is then detected in solution following displacement via e.g. ELISA reaction, or via fluorescent FABP probes that change their flouresence when binding free fatty acids (25). A drug candidate capable of displacing LA from its binding pocket. The immobilized SARS-CoV-2 S protein in this example can comprise the trimeric S protein, or a fragment or mutant thereof, wherein said fragment or mutant at least contains a receptor binding domain of said coronavirus S protein.

FIG. 9: Example 8. Screening method for discovery, i.e. identification, of inhibitors of LA binding to SARS-CoV-2 S protein. Shown is a schematic diagram describing a process to discover potential small molecule or biologic drug candidates which are inhibitors of LA binding to SARS-CoV-2 S protein. Left: indicated schematically is SARS-CoV-2 S protein immobilized on to the surface of a microplate in aqueous solution, optionally containing a carrier protein for LA such as BSA or a FABP. Indicated by the black rectangle is the LA binding pocket, which in this embodiment is occupied by a labelled version of LA indicated as LA*. A labelled LA could be e.g. a conjugated fluorophore derivative of LA. Center: a small molecule or biologic drug candidate is added to the solution of immobilized SARS-CoV-2 S protein. Right: LA* is displaced and replaced by drug candidate in the LA binding pocket. LA* is then detected directly in solution following displacement via its label group. The immobilized SARS-CoV-2 S protein in this example can comprise the trimeric S protein, or a fragment or mutant thereof, wherein said fragment or mutant at least contains a receptor binding domain of said coronavirus S protein.

FIG. 10: Example 9. Screening method for discovery, i.e. identification, of inhibitors of LA binding to SARS-CoV-2 S protein. Shown is a schematic diagram describing a high throughput-compatible process to discover potential small molecule or biologic drug candidates which are inhibitors of LA binding to SARS-CoV-2 S protein. Left: indicated schematically SARS-CoV-2 S protein is immobilized on to the surface of a microplate in aqueous solution optionally containing a carrier protein for LA such as BSA or a FABP. Indicated by the black rectangle is the LA binding pocket which is occupied by LA. Center: LA is stripped from SARS-CoV-2 S protein, and a molar excess of small molecule or biologic drug candidate is added to the solution of immobilized SARS-CoV-2 S protein, together with labeled LA indicated as LA*. As an alternative to stripping LA from SARS-CoV-2 S protein, a sample of the protein produced in the absence of LA could be used to produce the Apo form of the protein. Right: LA* and drug candidate compete for the LA binding pocket. The ratio of bound to unbound LA* is LA is then detected in solution via the label group of LA*. The immobilized SARS-CoV-2 S protein in this example can comprise the trimeric S protein, or a fragment or mutant thereof, wherein said fragment or mutant at least contains a receptor binding domain of said coronavirus S protein.

FIG. 11: Example 10. Screening method for discovery, i.e. identification, of inhibitors of LA binding to SARS-CoV-2 S protein. Shown is a schematic diagram describing a high throughput-compatible process to discover potential small molecule or biologic drug candidates which are inhibitors of LA binding to SARS-CoV-2 S protein. Left: indicated schematically SARS-CoV-2 S protein is immobilized on to the surface of a microplate in aqueous solution optionally containing a carrier protein for LA such as BSA or a FABP. Indicated by the black rectangle is the LA binding pocket which is not occupied by LA. Right: LA and drug candidate compete for the LA binding pocket. LA is then detected in solution via e.g. ELISA reaction, or via fluorescent FABP probes that change their flouresence when binding free fatty acids (25). The immobilized SARS-CoV-2 S protein in this example can comprise the trimeric S protein, or a fragment or mutant thereof, wherein said fragment or mutant at least contains a receptor binding domain of said coronavirus S protein.

FIG. 12: Example 11. Screening method for discovery, i.e. identification, of inhibitors of the Holo SARS-CoV-2 S protein—ACE2 interaction. Shown is a schematic diagram describing a medium/high throughput-compatible process to discover potential small molecule or biologic drug candidates which are inhibitors of the Holo SARS-CoV-2 S protein—ACE2 interaction. Left: indicated schematically is the ELISA assay from FIG. 6. Holo SARS-CoV-2 S protein and Labeled RBD-HRP* are incubated together at concentrations that produce approximately EC30 signal in the immobilized ACE2 receptor binding ELISA assay. Center: Small molecule or biologic drug candidate is added to the EC30 component mixture from left. Right: The ELISA reaction is run as described in FIG. 6 at single point readout at the putative EC30 of an inactive drug candidate. An increase of A450 is indicative that the drug candidate is an inhibitor of binding of SARS-CoV-2 S protein to ACE2. This method is anticipated to be able to detect therapeutic drug candidate inhibitors which bind SARS-CoV-2 S protein outside it's LA binding pocket. The SARS-CoV-2 S protein in this example can comprise the trimeric S protein, or a fragment or mutant thereof, wherein said fragment or mutant at least contains a receptor binding domain of said coronavirus S protein.

FIG. 13: Example 12. Screening method for discovery, i.e. identification, of Halo-specific or Apo-specific inhibitors of the Holo SARS-CoV-2 S protein—ACE2 interaction. Shown is a schematic diagram describing a medium/high throughput-compatible process to discover potential small molecule or biologic drug candidates which are inhibitors of the SARS-CoV-2 S protein—ACE2 interaction that are selective for either Apo or Halo form of the SARS-CoV-2 S protein. Left: indicated schematically is the ELISA assay from FIG. 6. Either Holo (top), or Apo (bottom) SARS-CoV-2 S protein and Labeled RBD-HRP* are incubated together at concentrations that produce approximately EC30 signal in the immobilized ACE2 receptor binding ELISA assay. Center: Small molecule or biologic drug candidate is added to the EC30 component mixture from left, to Holo mixture (top), or Apo mixture (bottom), where said reaction is carried out in separate wells in the microplate. Right: The ELISA reaction is run as described in FIG. 6 at single point readout at the putative EC30 of an inactive drug candidate, and the readout from Holo (top), or Apo (bottom) are compared for each putative drug candidate. An increase of A450 is indicative that the drug candidate is an inhibitor of binding of SARS-CoV-2 S protein to ACE2. This method is anticipated to be able to detect therapeutic drug candidate inhibitors which bind SARS-CoV-2 S protein outside it's LA binding pocket, and also to discern inhibitors that might have specificity for Holo or Apo form of SARS-CoV-2 S protein. The immobilized SARS-CoV-2 S protein in this example can comprise the trimeric S protein, or a fragment or mutant thereof, wherein said fragment or mutant at least contains a receptor binding domain of said coronavirus S protein.

FIG. 14: Example 13. Screening method for discovery, i.e. identification, of Halo-specific or Apo-specific inhibitors of the Holo SARS-CoV-2 S protein using the SARS-CoV-2 Spike receptor binding domain (RBD) or variants of the RBD containing one or several mutations. Depicted are zoom-ins on the LA-binding ‘greasy’ tube entrance within the receptor binding domain of the SARS-CoV-2 S protein. The RBD is shown in a cartoon representation. Bound LA is represented by spheres. Amino acid residues in proximity of the carboxy headgroup of LA, one each in the individual zoom-ins, are represented by spheres and marked in the amino acid sequence segments (from primary sequence of SARS-CoV-2 S) above the zoom-ins in bold and by enlarged fonts. The screening methods of any of the Examples 1 to 6 could optionally utilize SARS-CoV-2 S RBD proteins comprising one or more mutations where either of the featured amino acids, or combinations thereof, of all of them, have been changed to a hydrophilic amino acid, for example Lys (K), Arg (R), Asn (N) or (Gln) (Q), or other hydrophilic amino acids including non-natural amino acids. The said RBD variants are then used in this Example in medium/high throughput-compatible process to discover potential small molecule or biologic drug candidates which interact with high affinity and high specificity with the RBD proteins described. These small molecules or biologic drug candidates are selective for either Apo or Halo form of the SARS-CoV-2 S protein.

FIG. 15: Example 14. Discovery, i.e. identification, of Halo-specific or Apo-specific inhibitors of the Holo SARS-CoV-2 S protein using crystallization of the SARS-CoV-2 Spike RBD or variants of the RBD. The screening method in this example utilizes SARS-CoV-2 S protein RBD and/or the variants described in Example 13 (FIG. 14) in low/medium/high throughput-compatible crystallization and X-ray diffraction experiments including addition of one or several small molecule or biologic drug candidates in the crystallization experiments, to discover potential small molecule or biologic drug candidates which interact with high affinity and high specificity with the RBD proteins described. These small molecules or biologic drug candidates are selective for either Apo (top) or Holo form (bottom) of the SARS-CoV-2 S protein.

FIG. 16: Example 15. Discovery, i.e. identification, of Halo-specific or Apo-specific inhibitors of the Holo SARS-CoV-2 S protein using crystallization of the SARS-CoV-2 Spike RBD or variants of the RBD. Depicted is a zoom-in on Arginine R408 and Glutamine Q409 in the SARS-CoV-2 S protein. R408 and Q409 are part of the LA binding pocket in the Spike RBD. Spike RBD is shown in a cartoon representation. Bound LA is represented by sticks. R408 and Q409 are marked. The screening method in this embodiment utilizes SARS-CoV-2 S protein in which R408, or Q409, or both have been mutated to a different amino acid residue, preferentially an alanine or a glycine, or any other amino acid residue that is not polar and/or positively charged. The said SARS-CoV-2 S protein variants are then used in this Example in medium/high throughput-compatible crystallization process as described in Example 14 to discover potential small molecule or biologic drug candidates which interact with high affinity and high specificity with the SARS-CoV-2 S proteins described. These small molecules or biologic drug candidates are selective for either Apo or Halo form of the SARS-CoV-2 S protein.

FIG. 17: Example 17. Phage display screening method. Shown is a schematic diagram describing phage display applied to discover antibodies that bind to holo (LA bound) SARS-CoV-2 spike protein. Left: indicated schematically SARS-CoV-2 spike protein is immobilized on to the surface of a microplate in aqueous solution. Indicated by the black rectangle is the LA binding pocket which is occupied by LA. Left Arrow: Indicated by the Y-shaped images, a phage display library is incubated with the immobilized SARS-CoV-2 spike protein with some antibodies binding to SARS-CoV-2 spike protein (Center). Non interacting phage display library species are washed away leading to enriched samples of SARS-CoV-2 spike protein with preferred antibody binding partners (Right). Not shown: standard and iterative phage display washing/panning cycles and nucleic acid identification methods are used to develop and identify high affinity binders to (LA bound) SARS-CoV-2 spike protein.

FIG. 18: High affinity binding of linoleic acid (LA) to immobilized SARS-CoV-2 Spike Protein receptor binding domain (RBD) quantified by surface plasmon resonance (SPR). LA was diluted to concentrations between 4 μM and 10 μM and flowed over 3,800 RU of biotinylated and lipidex-treated RBD immobilized on a streptavidin-coated sensor chip (light grey tracings). Dark grey lines correspond to a global fit of the data using a 1:1 binding model. Each experiment was repeated independently three times. All LA concentrations were used to calculate the KD, kon and koff values.

FIG. 19: High affinity binding of Oleic Acid (OA) to immobilized SARS-CoV-2 Spike Protein receptor binding domain (RBD) quantified by surface plasmon resonance (SPR). OA was diluted to concentrations between 1 uM and 3 uM and flowed over 3,200 RU of biotinylated and lipidex-treated RBD immobilized on a streptavidin-coated sensor chip (light grey tracings). Dark grey lines correspond to a global fit of the data using a 1:1 binding model. Each experiment was repeated independently three times. All OA concentrations were used to calculate the KD, kon and koff values, which are indicated in this figure.

FIGS. 20A and 20B: Cryo-EM structure of the SARS-CoV-2 Spike protein, oleic acid complex. FIG. 20A: Cryo-EM density of the Spike trimer is shown from a side view (left), and top view (right). Bound oleic acid is illustrated in the top view as spheres. FIG. 20B: Composite oleic acid binding pocket formed by adjacent Receptor Binding Domains (RBDs). Tube-shaped EM density is shown.

FIGS. 21A-21C: Minivirus displaying SARS-CoV-2 Spike protein on its surface is blocked from binding its receptor ACE2 by linoleic acid. FIG. 21A: Schematic illustration of a Minivirus with SARS-CoV-2 Spike protein on its surface, immobilized via their His-tag. FIG. 21B: Representative confocal microscopy images of MCF7 human epithelial cells incubated for 10 min with Miniviruses, showing attachment of the Miniviruses to the cell's surface. Miniviruses are individually visualized as small dots. The inset shows magnified area of attachment. FIG. 21C: Miniviruses allowed for a systematic assessment of changes in SARS-CoV-2 Spike protein-mediated cell interactions as a function of Free Fatty Acid (FFA) occupancy. Because recombinant native SARS-CoV-2 Spike protein binds the Free Fatty Acids Linoleic Acid and Oleic Acid during heterologous protein expression and purification, we first generated FFA-depleted Spike protein (ApoS) by treatment of the purified SARS-CoV-2 Spike protein with lipidex columns as described in Example 17. Compared to the native Spike protein, ApoS-decorated Miniviruses displayed increased binding to human epithelial cells (First two bars on the left of the chart). This is consistent with a model where FFAs lock a closed Spike protein conformation, inhibiting binding to its receptor, ACE2. Controlled loading of ApoS Miniviruses by incubating them with FFAs of differing length and saturation (i.e., palmitic acid (PA), oleic acid (OA), LA and arachidonic acid (AA)). Binding of OA, and LA, but not PA, in SARS-CoV-2 Spike protein was successfully verified by multiple reactions monitoring LC-MS/MS. Of note, addition of saturated PA did not significantly reduce Minivirus-ACE2 cell binding compared to ApoS. However, we found that the (poly)-unsaturated FFAs OA, LA and AA were able to reduce Minivirus binding compared to ApoS Miniviruses.

FIG. 22A and FIG. 22B: Electron tomography of cultured mammalian cells infected with live SARS-CoV-2 virus, in the presence or absence of linoleic acid. A human gut epithelial cell line Caco2 expressing ACE2 (Caco-2-ACE2) was infected with SARS-CoV-2 reporter virus (for details see Example 21). Tomographs were obtained in the absence (FIG. 22A), or presence (FIG. 22B) of 50uM linoleic acid. Clearly visible in untreated cells (FIG. 22A) are the presence of enveloped “replication factories” containing numerous SARS-CoV-2 virions. The dashed square in FIG. 22A (left image represents such a replication factory which is shown at higher magnification in FIG. 22A (right image. In FIG. 22B the same SARS-CoV-2 infected cells were co-incubated with 50uM linoleic acid. The dashed square in FIG. 22B (left image) is the site of a former SARS-CoV-2 replication factory. The dashed square in FIG. 22B(left image) reveals that linoleic acid penetrates the cells, forming lipid droplets inside the cell, which are well tolerated by the cell (significant cell death was not observed). At higher magnification in FIG. 22B (right image) it is revealed that the envelope of the former “replication factory” has been disrupted, and the viral particles themselves suffering visible deformation and destruction.

### DETAILED DESCRIPTION OF THE INVENTION

The present invention is further illustrated by the following non-limiting examples:

### EXAMPLES

### Example 1

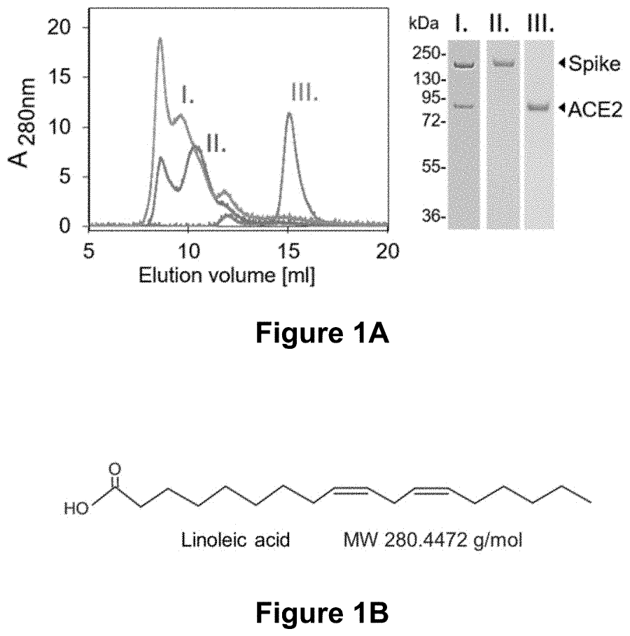

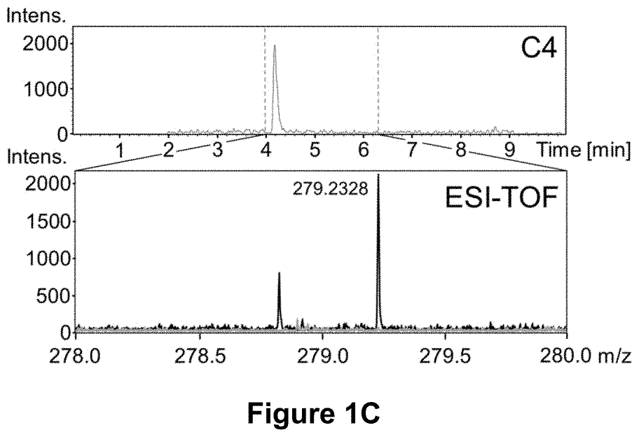

SARS-CoV-2 S protein production, with bound LA. The pFastBac Dual plasmid for SARS-CoV-2 S ectodomain for expression in insect cells 20 was kindly provided by Florian Krammer (Icahn School of Medicine, USA). In this construct, S comprises amino acids 1 to 1213 and is fused to a C-terminal thrombin cleavage site followed by a T4-foldon trimerization domain and a hexahistidine affinity purification tag. The polybasic cleavage site has been deleted (RRAR to A) in the construct 20. Protein was produced with the MultiBac baculovirus expression system (Geneva Biotech, Geneva, Switzerland) 40 in Hi5 cells using ESF921 media (Expression Systems Inc.). Supernatants from transfected cells were harvested 3 days post-transfection by centrifugation of the culture at 1,000g for 10 min followed by another centrifugation of supernatant at 5,000g for 30 min. The final supernatant was incubated with 7 ml HisPur Ni-NTA Superflow Agarose (Thermo Fisher Scientific) per 3 litres of culture for 1 h at 4° C. Subsequently, a gravity flow column was used to collect the resin bound with SARS-CoV-2 S protein, the resin was washed extensively with wash buffer (65 mM NaH2PO4, 300 mM NaCl, 20 mM imidazole, pH 7.5), and the protein was eluted using a step gradient of elution buffer (65 mM NaH2PO4, 300 mM NaCl, 235 mM imidazole, pH 7.5). Elution fractions were analysed by reducing SDS-PAGE and fractions containing SARS-CoV-2 S protein were pooled, concentrated using 50 kDa MWCO Amicon centrifugal filter units (EMD Millipore) and buffer-exchanged in phosphate-buffered saline (PBS) pH 7.5. Concentrated SARS-CoV-2 S was subjected to size exclusion chromatography (SEC) using a Superdex 200 increase 10/300 column (GE Healthcare) in PBS pH 7.5. Peak fractions from SEC were analysed by reducing SDS-PAGE (See FIG. 1A).

Mass spectrometry analysis of SARS-CoV-2 S protein production, demonstrating bound LA. For mass spectrometry analysis, a Bruker maXis II ETD quadrupole-time-of-flight instrument with electrospray ionization (ESI) coupled to a Shimadzu Nexera HPCL was used. In order to extract the fatty acids from the protein sample, a chloroform extraction protocol was performed. For this, 100 μl of the protein sample was mixed with 400 pl chloroform for 2 hours on a horizontal shaker in a teflon-sealed glass vial at 25° C. The organic phase was then transferred to a new glass vial and the chloroform was evaporated for 30 min in a desiccator. Subsequently, 50 μl of a 20% (v/v) acetonitrile solution was added to dissolve the fatty acids. From this solution, a 1:100 dilution in 20% (v/v) acetonitrile was prepared and 20 μl were injected for LC-MS analysis. The samples were passed over a Phenomenex C4 Aeris column (100×2.1 mm, 3.6μ, 200 Å) heated to 50° C. using a gradient of 20% A to 98% B in 5 min (solvent A: H₂O+0.1% formic acid; solvent B: ACN+0.1% formic acid). The system was operated with Bruker's O-TOF Control (V4.1) and Hystar (V4.1) software and data analysis as well as data post-processing was performed with Bruker's Data Analysis software (V4.4, SR1, See FIG. 1C).

### Example 2

ACE2 protein production. The gene encoding for the ACE2 ectodomain (amino acids 1 to 597, 41) was codon optimized for insect cell expression, synthesized (Genscript Inc, New Jersey USA) and inserted into pACEBac1 plasmid (Geneva Biotech, Geneva, Switzerland). The construct contains an N-terminal melittin signal sequence for secretion and a C-terminal octahistidine affinity purification tag. ACE2 protein was produced and purified following the same protocol as for S protein (See FIG. 1A).

### Example 3

SARS-CoV-2 Spike and ACE2 interaction analysis. Purified SARS-CoV-2 Spike and ACE2 proteins were combined in PBS pH 7.5 at a ratio of 1:1.5 Spike trimer to ACE2 monomer. The mixture was incubated on ice for 2 hours and subjected to SEC using a Superdex 200 increase 10/300column (GE Healthcare) equilibrated in PBS pH 7.5. Purified SARS-CoV-2 S protein and ACE2 proteins were individually run on the same column in the same buffer as controls. The contents of peak fractions were analyzed by reducing SDS-PAGE (See FIG. 1A).

### Example 4

Cryo-EM Image Production.

Sample preparation and data collection. 4 μL of 1.25 mg/mL SARS-CoV-2 S protein was loaded onto a freshly glow discharged (2 min at 4 mA) Quantifoil R1.2/1.3 carbon grid (Agar Scientific), blotted using a Vitrobot MarklV (Thermo Fisher Scientific) at 100% humidity and 4° C. for 2 s, and plunge frozen. Data were acquired on a FEI Talos Arctica transmission electron microscope operated at 200 kV and equipped with a Gatan K2 Summit direct detector and Gatan Quantum GIF energy filter, operated in zero-loss mode with a slit width of 20 eV using the EPU software.

Data were collected in super-resolution at a nominal magnification of 130,000× with a virtual pixel size of 0.525 Å. The dose rate was adjusted to 6.1 counts/physical pixel/s. Each movie was fractionated in 55 frames of 200 ms. 3289 micrographs were collected in a single session with a defocus range comprised between −0.8 and −2 μm.

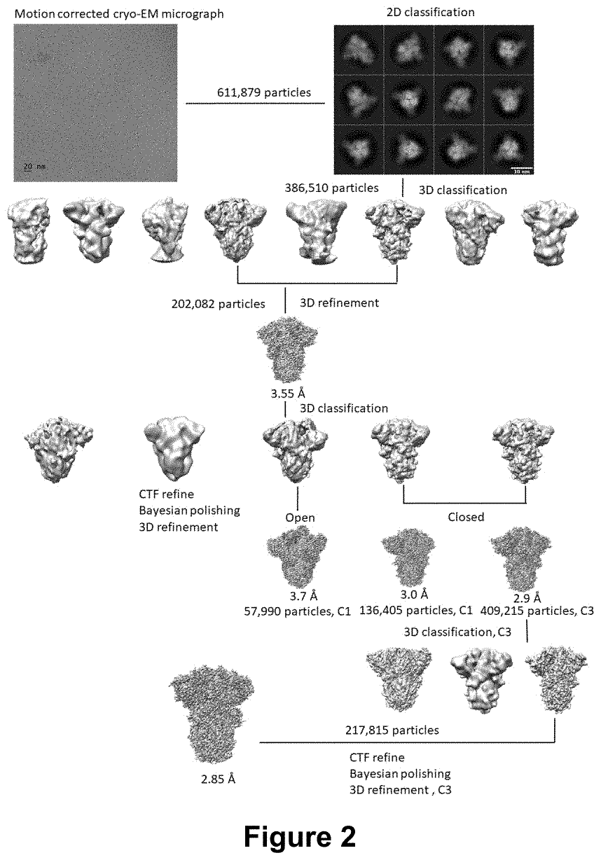

Data Processing. The dose-fractionated movies were gain-normalised, aligned, and dose-weighted using MotionCor2 42. Defocus values were estimated and corrected using CTFFIND4 43. 611,879 particles were automatically picked using Relion 3.0 software 44. Reference-free 2D classification was performed to select well-defined particles, after four rounds of 2D classification a total of 386,510 good particles were selected for further 3D classification. An initial model was generated using 50,000 particle images in Relion 3.0, and the selected particles from 2D classification were subjected to 3D classification using 8 classes. Classes 4 and 6 (see FIG. 2), showing prominent features representing a total of 202,082 particles were combined and used for 3D refinement. The 3D refined particles were then subjected to a second round of 3D classification using 5 classes. Class 4 and 5 were combined for the closed conformation map, class 3 represented the open conformation, comprising 136,405 and 57,990 particles respectively. The selected maps were subjected to 3D refinement without applying any symmetry with respective 3D models. The maps were subsequently subjected to local defocus correction and Bayesian particle polishing in Relion 3.0. Global resolution and B factor (−89Å2 and −116Å2 for closed and open maps respectively) of the map were estimated by applying a soft mask around the protein density, using the gold standard Fourier shell correlation (FSC) =0.143 criterion, resulting in an overall resolution of 3.03 Å and 3.7 Å respectively. C3 symmetry was applied to the closed conformation map using Relion 3.0, followed by 3D classification using 3 classes. Class 3 with 217,815 particles was selected for CTF refinement and Bayesian polishing, yielding a final resolution of 2.85 Å (B factor of −86.8). Local resolution maps were generated using Relion 3.0.

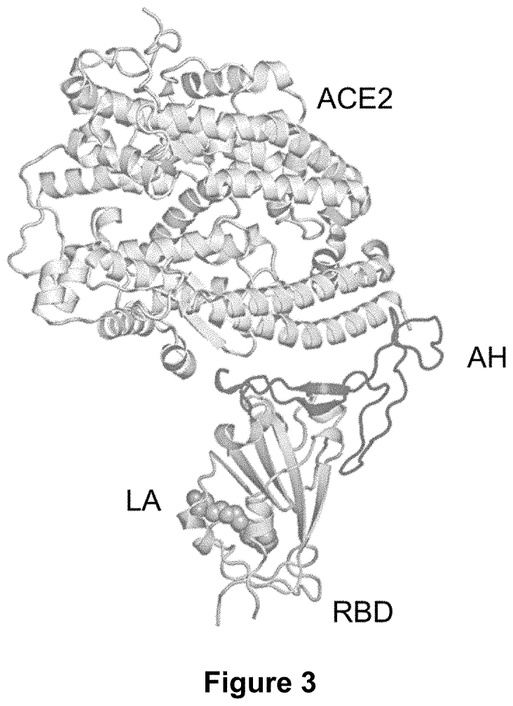

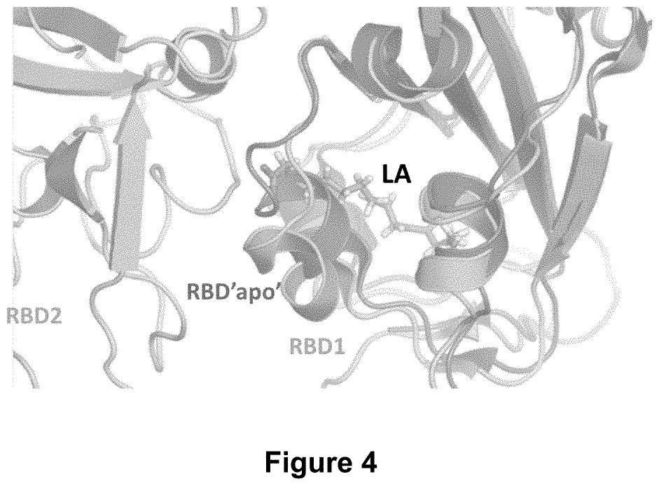

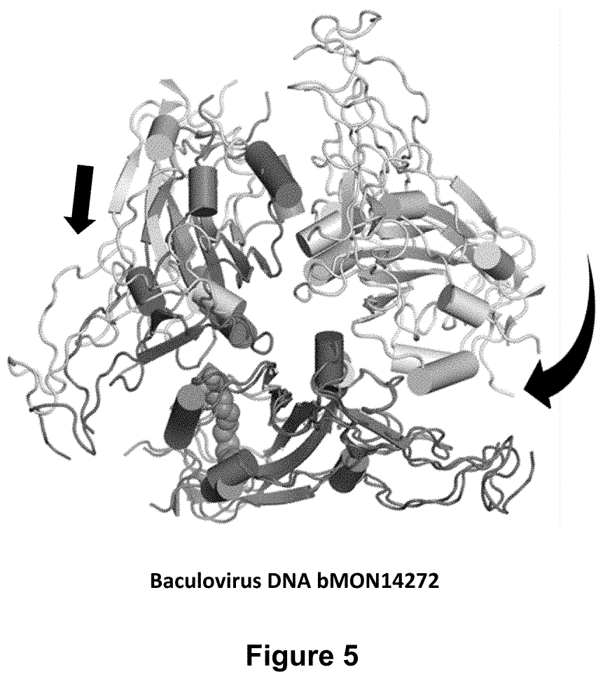

Cryo-EM model building and analysis. UCSF Chimera 45 and Coot 46 were used to fit atomic models (PDB ID 6VXX, 14) into the cryo-EM map. The model was subsequently manually rebuilt using Coot and the closed conformation map. This closed conformation model was used to build features specific of the open conformation map and to further improve the model by using the C3-symmetrised map. To guide the model building, sharpened 47 and unsharpened maps were used in different steps of the process. N-linked glycans were hand-built into the density where visible, and restraints for non-standard ligands were generated with eLBOW 48. The model for the closed conformation was real space refined with Phenix 49 and the quality was additionally analyzed using MolProbity 50 and EMRinger 51, to validate the stereochemistry of the components. The quasi-atomic model of the open conformation was generated by first fitting the atomic model of the closed conformation into the open cryo-EM map using UCSF Chimera. We then used COOT and UCSF Chimera to move one RBD into the open conformation, by aligning this RBD to the atomic model of a published open form of SARS-CoV-2 S (PDB ID 6VSB, 24). Finally, this open model was fitted into the cryo-EM open map with COOT and UCSF Chimera. Figures were prepared using UCSF chimera and PyMOL (Schrodinger, Inc). The resulting Cryo-EM model building enabled us to determine binding site of LA in the SARS-CoV-2 S protein, to identify conformational changes upon LA binding, and also to model SARS-CoV-2 S protein—ACE2 receptor interactions (See FIGS. 3, 4, and 5).

### Example 5

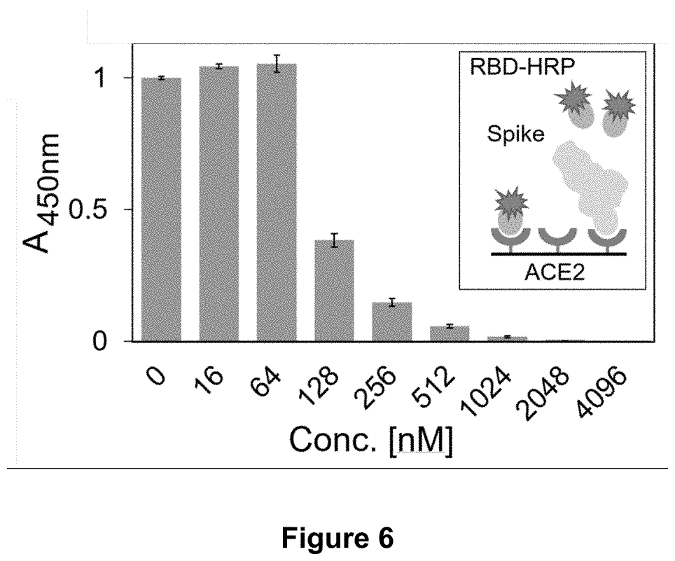

ELISA activity assay, illustrated in FIG. 6. The SARS-CoV-2 sVNT kit was obtained from GenScript Inc. (New Jersey, USA) for ELISA activity assays. Serial dilution series were prepared (from 0-4096 nM) of purified SARS-CoV-2 S protein in PBS pH 7.5. This dilutions series was mixed with the same amount of HRP-RBD and incubated at 37° C. for 30 min. The mixture of each dilution of SARS-CoV-2 S proteins and HRP-RBD was then added to ELISA plate wells, which were coated with ACE2. Triplicates of each sample were added. The ELISA plate was then incubated at 37° C. for 15 min and subsequently washed 4 times with wash buffer. The signal was then developed by adding TMB solution to each well, incubating 15 minutes in the dark, followed by adding stop solution. The absorbance at 450 nm was immediately recorded. The data was plotted using Microsoft excel. The standard deviation of triplicates was added as error bars. This Example illustrates the functionality of our SARS-CoV-2 S protein in functional biochemical assays which are applicable to drug discovery, i.e. identification.

### Example 6

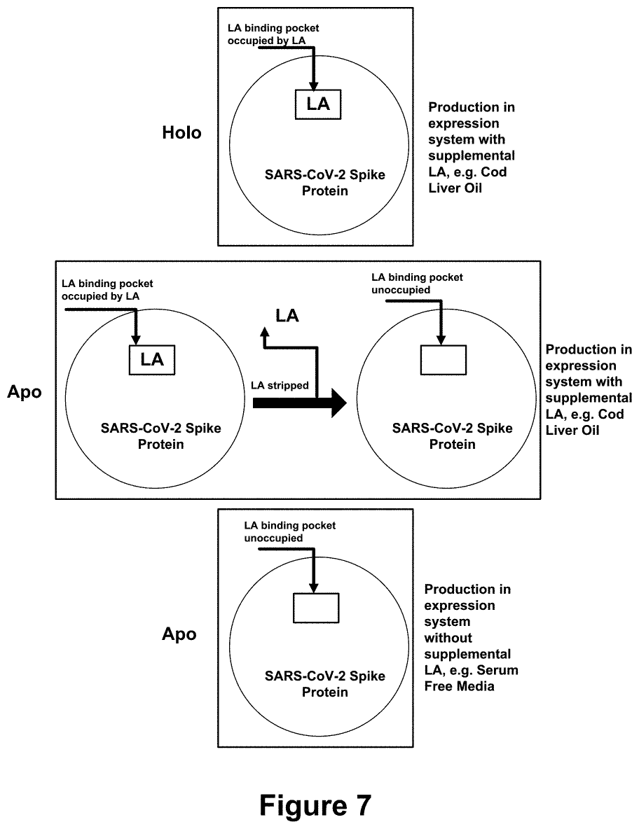

FIG. 7 illustrates production methods for purified SARS-CoV-2 S protein. Shown is a schematic diagram illustrating 3 general strategies for producing either Holo (meaning LA-containing) SARS-CoV-2 S protein, or Apo (meaning not LA-containing) SARS-CoV-2 S protein, respectively. By means of these processes, Apo and Holo can be produced for side by side comparison in drug discovery, i.e. identification, processes targeting SARS-CoV-2 S protein in the below Examples.

### Example 7

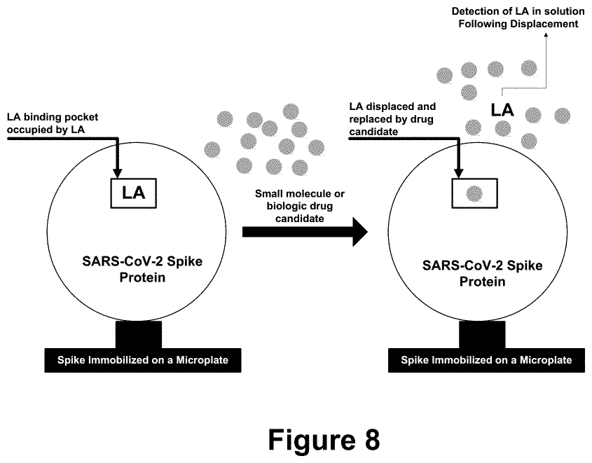

FIG. 8 illustrates a screening method for discovery, i.e. identification, of inhibitors of LA binding to SARS-CoV-2 S protein. Shown is a schematic diagram describing a high throughput-compatible process to discover potential small molecule or biologic drug candidates which are inhibitors of LA binding to SARS-CoV-2 S protein. In this method a SARS-CoV-2 S protein, or a fragment or mutant thereof wherein said fragment or mutant at least contains a receptor binding domain of said coronavirus S protein, is immobilized on to the surface of a microplate in aqueous solution optionally containing a carrier protein for LA such as BSA or Fatty Acid-Binding Protein (FABP). In this example, SARS-CoV-2 S protein, or a fragment or mutant thereof is bound by LA. A molar excess of a small molecule or biologic drug candidate is added to the solution of immobilized SARS-CoV-2 S protein (or a fragment or mutant thereof) with bound LA. In case of a bona fide inhibitor drug candidate, LA could be displaced and replaced by the drug candidate in the LA binding pocket. LA has extremely low water solubility, and BSA is often used as a carrier protein to provide aqueous bioavailability of LA and other free fatty acids. In the experimental setup of Example 7 which includes a carrier protein for LA such as BSA or a FABP, LA that is displaced from SARS-CoV-2 S protein by the drug candidate will have a new binding site available in solution provided by carrier protein BSA or FABP. LA can be detected in solution following displacement via e.g. ELISA reaction directly detecting LA, or, alternatively by the conformational change-driven change in FABP fluorescence induced by LA binding (25).

### Example 8

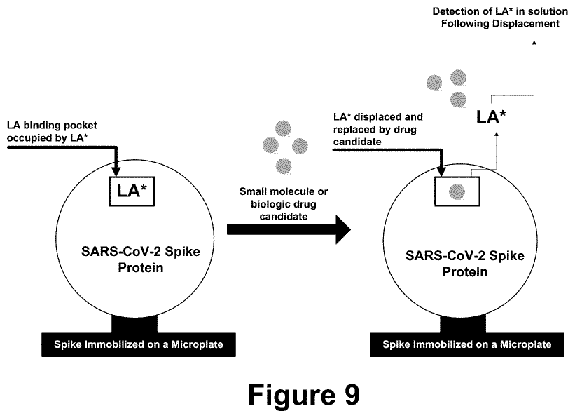

FIG. 9 illustrates a screening method for discovery, i.e. identification, of inhibitors of LA binding to SARS-CoV-2 S protein. Shown is a schematic diagram describing a high throughput-compatible process to discover potential small molecule or biologic drug candidates which are inhibitors of LA binding to SARS-CoV-2 S protein. In this method a SARS-CoV-2 S protein, or a fragment or mutant thereof wherein said fragment or mutant at least contains a receptor binding domain of said coronavirus S protein, is immobilized on to the surface of a microplate in aqueous solution optionally containing a carrier protein for LA such as BSA or Fatty Acid-Binding Protein (FABP). In this example, SARS-CoV-2 S protein, or a fragment or mutant thereof is bound by a labelled analogue of LA, indicated as LA*. A molar excess of a small molecule or biologic drug candidate is added to the solution of immobilized SARS-CoV-2 S protein (or a fragment or mutant thereof) with bound LA*. In case of a bona fide inhibitor drug candidate, LA* could be displaced and replaced by the drug candidate in the LA binding pocket. In the experimental setup of Example 8 which includes a carrier protein for LA such as BSA or a FABP, LA* that is displaced from SARS-CoV-2 S protein by the drug candidate will have a new binding site available in solution provided by carrier protein BSA or FABP. LA* can be detected in solution following displacement via its label, e.g. a fluorescent label.

### Example 9

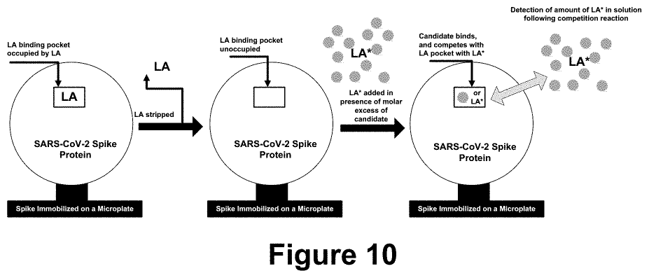

FIG. 10 illustrates a screening method for discovery, i.e. identification, of inhibitors of LA binding to SARS-CoV-2 S protein. Shown is a schematic diagram describing a high throughput-compatible process to discover potential small molecule or biologic drug candidates which are inhibitors of LA binding to SARS-CoV-2 S protein. In this method a SARS-CoV-2 S protein, or a fragment or mutant thereof wherein said fragment or mutant at least contains a receptor binding domain of said coronavirus S protein, is immobilized on to the surface of a microplate in aqueous solution optionally containing a carrier protein for LA such as BSA or Fatty Acid-Binding Protein (FABP). In this example, SARS-CoV-2 S protein, is first stripped of bound LA, or produced by a method that does not result in bound LA (see Example 6). Next, a molar excess of a small molecule or biologic drug candidate is added to the solution of immobilized SARS-CoV-2 S protein (or a fragment or mutant thereof) together with a labelled LA, indicated as LA*, whereby LA* is optionally provided in complex with a carrier protein for LA* such as BSA or FABP. In case of a bona fide inhibitor drug candidate, the quantity of LA* bound to the LA binding pocket will be decreased. LA* can be detected in solution or bound to the SARS-CoV-2 S protein following the competition reaction via its label, e.g. a fluorescent label.

### Example 10

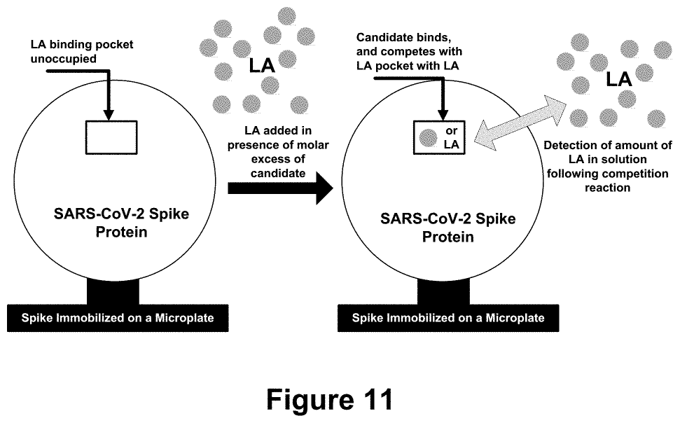

FIG. 11 illustrates a screening method for discovery, i.e. identification, of inhibitors of LA binding to SARS-CoV-2 S protein. Shown is a schematic diagram describing a high throughput-compatible process to discover potential small molecule or biologic drug candidates which are inhibitors of LA binding to SARS-CoV-2 S protein. In this method a SARS-CoV-2 S protein, or a fragment or mutant thereof wherein said fragment or mutant at least contains a receptor binding domain of said coronavirus S protein, is immobilized on to the surface of a microplate in aqueous solution optionally containing a carrier protein for LA such as BSA or Fatty Acid-Binding Protein (FABP). In this example, SARS-CoV-2 S protein, is first stripped of bound LA, or produced by a method that does not result in bound LA (see Example 6). Next, a molar excess of a small molecule or biologic drug candidate is added to the solution of immobilized SARS-CoV-2 S protein (or a fragment or mutant thereof) together with a unlabelled LA, indicated as LA, whereby LA is optionally provided in complex with a carrier protein for LA such as BSA or FABP. In case of a bona fide inhibitor drug candidate, the quantity of LA bound to the LA binding pocket will be decreased. LA can be detected in solution or bound to the SARS-CoV-2 S protein following the competition reaction via e.g. ELISA reaction directly detecting LA, or, alternatively by the conformational change-driven change in FABP fluorescence induced by LA binding (25).

### Example 11

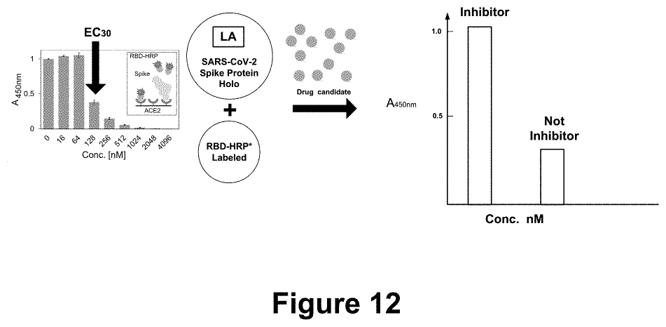

FIG. 12 illustrates a screening method for discovery, i.e. identification, of inhibitors of the Holo SARS-CoV-2 S protein—ACE2 interaction based on the ELISA assay from FIG. 6. Here, Holo SARS-CoV-2 S protein and Labelled RBD-HRP* are incubated together at concentrations that produce approximately EC30 signal in the immobilized ACE2 receptor binding ELISA assay. Small molecule or biologic drug candidate is added to the EC30 component mixture, and the ELISA reaction is run as described in FIG. 6 at single point readout at the putative EC30 of an inactive drug candidate. An increase of A450 is indicative that the drug candidate is an inhibitor of binding of SARS-CoV-2 S protein to ACE2. This method is anticipated to be able to detect therapeutic drug candidate inhibitors which bind SARS-CoV-2 S protein either inside, or outside its LA binding pocket.

### Example 12

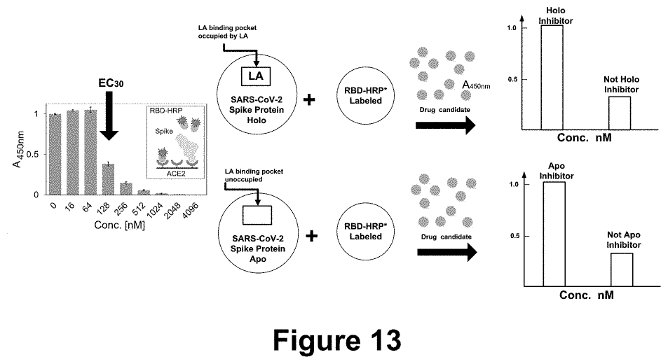

FIG. 13 illustrates a screening method for discovery, i.e. identification, of Halo-specific or Apo-specific inhibitors of the Holo SARS-CoV-2 S protein—ACE2 interaction based on the ELISA assay from FIG. 6. Here either Apo or Holo SARS-CoV-2 S protein and Labelled RBD-HRP* are incubated together at concentrations that produce approximately EC30 signal in the immobilized ACE2 receptor binding ELISA assay. Small molecule or biologic drug candidate is added to the EC30 component mixture, and the ELISA reaction is run as described in FIG. 6 at single point readout at the putative EC30 of an inactive drug candidate. An increase of A450 is indicative that the drug candidate is an inhibitor of binding of SARS-CoV-2 S protein to ACE2. This method is anticipated to be able to detect therapeutic drug candidate inhibitors which bind SARS-CoV-2 S protein either inside, or outside its LA binding pocket and also to discern inhibitors that might have specificity for Holo or Apo form of SARS-CoV-2 S protein.

### Example 13

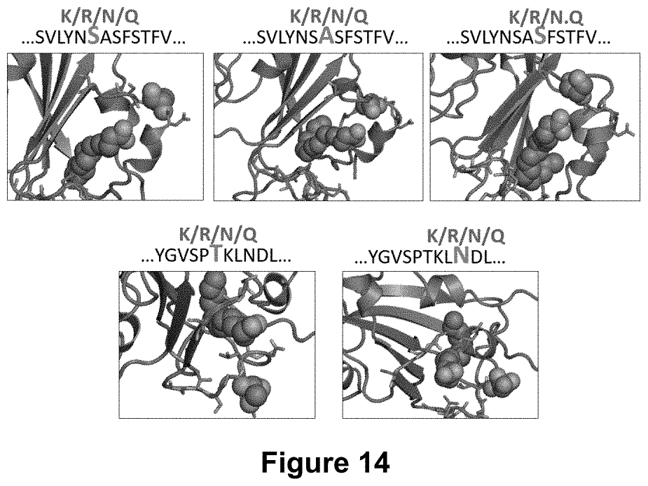

FIG. 14 illustrates a screening method for discovery, i.e. identification, of Halo-specific or Apo-specific inhibitors of the Holo SARS-CoV-2 S protein using the SARS-CoV-2 Spike receptor binding domain (RBD) or variants of the RBD containing one or several mutations. The screening methods of any of the Examples 1-6 could optionally utilize SARS-CoV-2 S RBD proteins comprising one or more mutations where either of the featured amino acids, or combinations thereof, of all of them, have been changed to a hydrophilic amino acid, for example Lys (K), Arg (R), Asn (N) or (Gln (Q), or other hydrophilic amino acids including non-natural amino acids. The said RBD variants are then used in this Example in medium/high throughput-compatible process to discover potential small molecule or biologic drug candidates which interact with high affinity and high specificity with the RBD proteins described. These small molecules or biologic drug candidates are selective for either Apo or Halo form of the SARS-CoV-2 S protein.

### Example 14

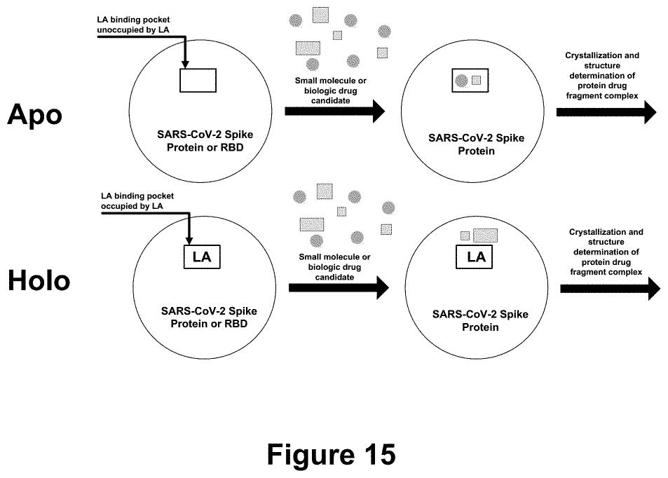

FIG. 15 illustrates a screening method for discovery, i.e. identification, of Halo-specific or Apo-specific inhibitors of the Holo SARS-CoV-2 S protein using crystallization of the SARS-CoV-2 Spike RBD or variants of the RBD. The screening method in this example utilizes SARS-CoV-2 S protein RBD and/or the variants described in Example 13 (FIG. 14) in low/medium/high throughput-compatible crystallization and X-ray diffraction experiments including addition of one or several small molecule or biologic drug candidates in the crystallization experiments, to discover potential small molecule or biologic drug candidates which interact with high affinity and high specificity with the RBD proteins described. Fragment-based screening (FBS) is a widely applied method for the discovery, i.e. identification, of drug candidates. It has overtaken high throughput screening (HTS) as the most popular method for screening molecules, due to the significantly fewer compounds required for screening and synthesis, resulting in a higher hit rate for screening molecules than traditional screening methods. Here, single compound or multiple compound mixtures are incubated with the target protein: SARS-CoV-2 S protein RBD and/or the variants described in Example 13 (FIG. 14), and crystallization is employed to identify binders. In this Example, small molecules or biologic drug candidates targeting the LA binding pocket which are selective for either Apo or Holo form of the SARS-CoV-2 S protein can be discovered.

### Example 15

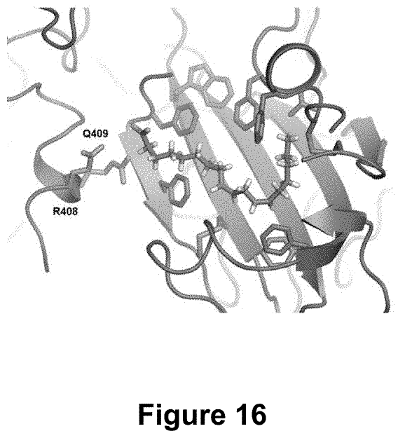

FIG. 16 illustrates a screening method for discovery, i.e. identification, of Halo-specific or Apo-specific inhibitors of the Holo SARS-CoV-2 S protein using crystallization of the SARS-CoV-2 Spike RBD or variants of the RBD. The screening method in this embodiment utilizes SARS-CoV-2 S protein in which R408, or Q409, or both have been mutated to a different amino acid residue, preferentially an alanine or a glycine, or any other amino acid residue that is not polar and/or positively charged. The said SARS-CoV-2 S protein variants are then used in this Example in medium/high throughput-compatible crystallization process as described in Example 14. These small molecules or biologic drug candidates are selective for either Apo or Halo form of the SARS-CoV-2 S protein.

### Example 16

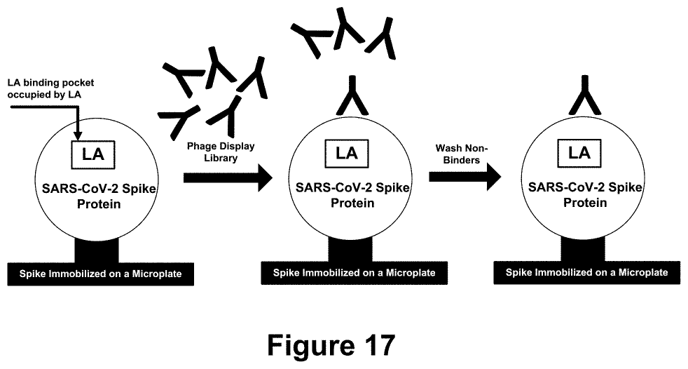

FIG. 17 illustrates a screening method of the invention for identifying binder molecules to complexes of the invention, wherein antibodies as binder molecules and phage display are used as examples for binding an immobilized exemplary complex of the invention composed SARS-CoV-2 S protein and LA. A library of potential binder molecules, here a phage display antibody library is incubated with the immobilized complex and one or more candidate binding antibodies are bound to the complex. Unbound binder molecules of the library are removed by a washing step which results in enriched samples of binder molecules of the complex of the invention with bound binder molecules. The process is typically embodied in an iterative fashion using a first enriched pool of binder molecule obtain in the first cycle is used in a further cycle of incubation and washing, where the washing and/or incubation step is carried out under conditions allowing only binding of such first cycle-enriched candidate binder molecules having a higher affinity as compared to the first cycle and so on.

### Example 17

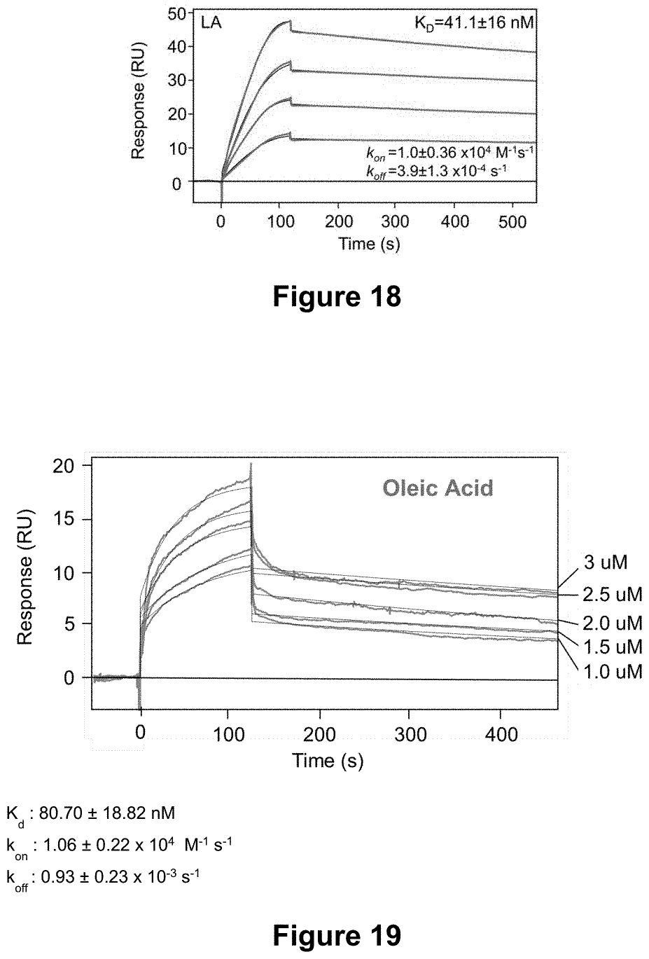

Example 17 illustrates the high affinity binding of Linoleic Acid (LA) to the SARS-CoV-2 Spike Protein. Shown in FIG. 18 is a Surface Plasmon Resonance (SPR) analysis of the binding of Linoleic Acid (LA) to the SARS-CoV-2 Spike Protein receptor binding domain (RBD).

The methods utilized to conduct the SPR binding assay are as follows:

Binding assay. LA was diluted to concentrations between 4 μM and 10 μM and flowed over 3,800 RU of biotinylated and lipidex-treated RBD immobilized on a streptavidin-coated sensor chip. Black lines correspond to a global fit of the data using a 1:1 binding model. Each experiment was repeated independently three times. All LA concentrations were used to calculate the KD, kon and koff values. The results are shown in FIG. 18.

Receptor Binding domain (RBD) and biotinylated RBD expression cassette design. The gene encoding for the SARS-CoV-2 RBD (amino acids R319 to F541), fused at its N-terminus to the native spike signal sequence (amino acid sequence MFVFLVLLPLVSSQ), was codon optimized for insect cell expression, synthesized (Genscript Inc., New Jersey USA) and inserted into pACEBac1 plasmid (Geneva Biotech, Geneva, Switzerland). The resulting construct pACEBac1-5 RBD HIS comprises a C-terminal octa-histidine affinity purification tag.

RBD Protein expression and purification. Protein was produced with the MultiBac baculovirus expression system (Geneva Biotech, Geneva, Switzerland) in Hi5 cells using ESF921 media (Expression Systems Inc.). Supernatants from transfected cells were harvested 3 days post-transfection by centrifugation of the culture at 1,000 g for 10 min followed by another centrifugation of supernatant at 5,000 g for 30 min. The final supernatant was incubated with 7 ml HisPur Ni-NTA Superflow Agarose (Thermo Fisher Scientific) per 3 liters of culture for 1 h at 4° C. Subsequently, a gravity flow column was used to collect the resin bound with SARS-CoV-2 RBD protein, the resin was washed extensively with wash buffer (65 mM NaH2PO4, 300 mM NaCl, 20 mM imidazole, pH 7.5), and the protein was eluted using a step gradient of elution buffer (65 mM NaH2PO4, 300 mM NaCl, 235 mM imidazole, pH 7.5). Elution fractions were analyzed by reducing SDS-PAGE and fractions containing SARS-CoV-2 RBD protein were pooled, concentrated using 50 kDa MWCO Amicon centrifugal filter units (EMD Millipore) and buffer-exchanged in phosphate-buffered saline (PBS) pH 7.5. Concentrated SARS-CoV-2 RBD protein was subjected to size exclusion chromatography (SEC) using a Superdex 200 increase 10/300 column (GE Healthcare) in PBS pH 7.5.

Biotinylation of RBD. Biotinylated RBD was generated by adding an avi tag for BirA mediated biotinylation as described in (M. Fairhead, M. Howarth, Site-specific biotinylation of purified proteins using BirA. Methods Mol. Biol. 1266, 171-184 (2015). doi:10.1007/978-1-4939-2272-7_12 Medline). Lipidex-treated biotinylated RBD was then prepared as described above for lipidex-treated S.

Lipidex-treatment of RBD protein to remove potential bound free fatty acid. Purified RBD protein was treated with lipidex-1000 resin (Perkin Elmer; cat no. 6008301), pre-equilibrated in PBS pH 7.5 overnight at 4 degrees C. on a roller shaker. Subsequently, lipidex-treated RBD was separated from the resin using a gravity flow column. Integrity of the protein was confirmed by size exclusion chromatography (SEC) using a S200 10/300 increase column (GE Healthcare) and SDS-PAGE.

### Example 18

In order to assess the binding characteristics of fatty acids related to linoleic acid, the experiments of Example 17 were repeated, but using oleic acid (OA) instead of linoleic acid (LA). OA was diluted to concentrations between 1 μM and 3 μM and flowed over 3,200 RU of biotinylated and lipidex-treated RBD immobilized on a streptavidin-coated sensor chip. Black lines correspond to a global fit of the data using a 1:1 binding model. Each experiment was repeated independently three times. All OA concentrations were used to calculate the KD, kon and koff values. The results are shown in FIG. 19.

### Example 19

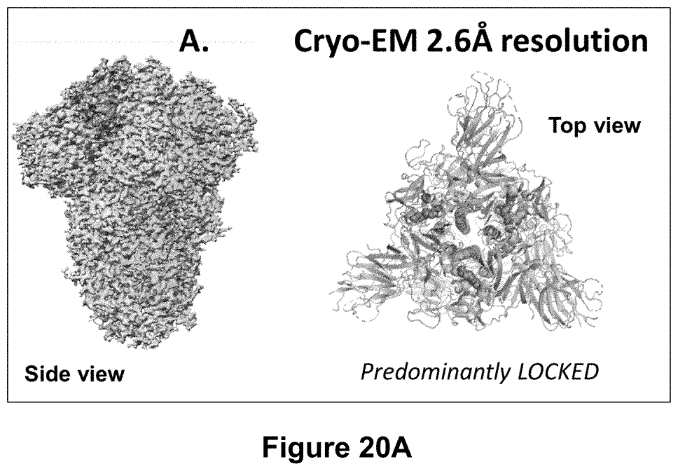

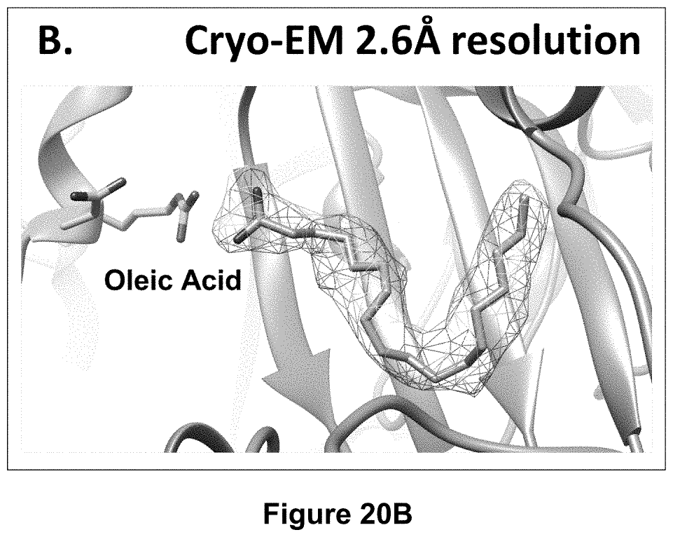

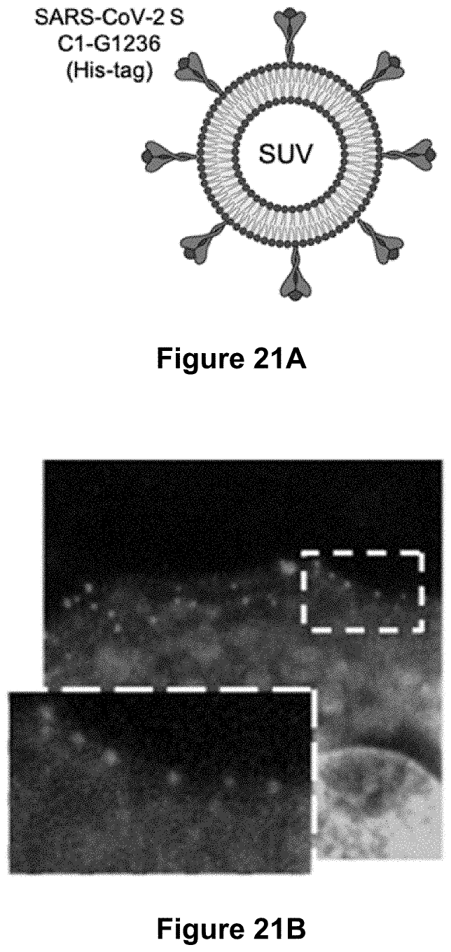

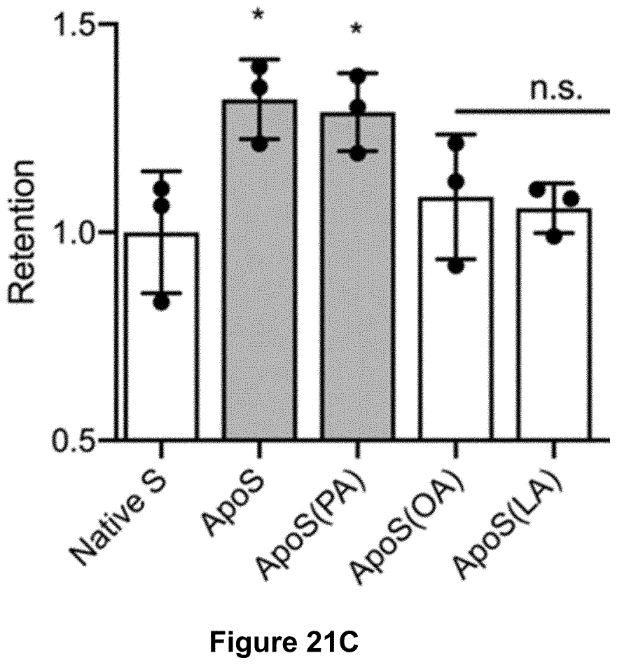

Example 19 reveals the precise binding mode between Oleic Acid and the SARS-CoV-2 Spike Protein. The structure of Oleic Acid bound to the SARS-CoV-2 Spike trimer was determined at 2.6 A resolution by cryo-electron miscroscopy. The results are shown in FIGS. 20A and 20B.

The methods utilized to determine the structure are as follows:

Protein expression and purification. Spike protein was expressed and purified as described (Toelzer et al Science 2020) except that minimal media devoid of supplemented free fatty acids was used (Expression system Inc). No FFA (OA or else) was supplemented at any step.

Cryo-EM sample preparation and data collection. 4 μL of ˜1 mg/mL Spike was loaded onto a freshly glow discharged (2 min at 4 mA) C-flat R1.2/1.3 carbon grid (Agar Scientific), blotted using a Vitrobot MarklV (Thermo Fisher Scientific) at 100% humidity and 4° C. for 2 s, and plunge frozen. Data were acquired on a FEI Talos Arctica transmission electron microscope operated at 200 kV and equipped with a Gatan K2 Summit direct detector and Gatan Quantum GIF energy filter, operated in zero-loss mode with a slit width of 20 eV using the EPU software. Data were collected in super-resolution at a nominal magnification of 130000× with a virtual pixel size of 0.525 A. The dose rate was adjusted to 6.1 counts/physical pixel/s. Each movie was fractionated in 55 frames of 200 ms. 8,639 micrographs were collected in a single session with a defocus range comprised between −0.8 and −2 μm.

Cryo-EM data processing. The dose-fractionated movies were gain-normalized, aligned, and dose-weighted using MotionCor2. Defocus values were estimated and corrected using the Gctf program. >1 Mio particles were automatically picked using Relion 3.0 software. The auto-picked particles were extracted with a box size of 110 px (2× binning). Reference-free 2D classification was performed to select well-defined particles. After four rounds of 2D classification >400.000 particles were selected for subsequent 3D classification. The initial 3D model was filtered to 60 A during 3D classification in Relion using 8 classes. A box size of 220 px (1.05 A/px, unbinned) was used. 3D-refined particles were then subjected to a second round of 3D classification yielding 200.000 particles. These particles were subjected to 3D refinement without applying any symmetry. The maps were subsequently subjected to local defocus correction and Bayesian particle polishing in Relion 3.1. Global resolution and B factor (−97.6 Å2) of the map were estimated by applying a soft mask around the protein density, using the gold-standard Fourier shell correlation (FSC) =0.143 criterion, resulting in an overall resolution of 3.0 A. C3 symmetry was applied to the Bayesian polished C1 map using Relion 3.1 yielding a final resolution of 2.6 A (B factor of −106.7 Å2).

Cryo-EM model building and analysis. For model building, UCSF Chimera was used to fit an atomic model of the SARS-CoV2 Spike locked conformation (Toelzer et al Science 2020) into the C3 symmetrized cryo-EM map. The model was rebuilt using sharpened and unsharpened maps in Coot [and then fitted into the C1 cryo-EM map. Namdinator and Coot were used to improve the fit and N-linked glycans were built into the density for both models where visible. Restraints for non-standard ligands were generated with eLBOW. The model for C1 and C3-symmetrized closed conformation was real space refined with Phenix, and the quality was additionally analyzed using MolProbity and EMRinger, to validate the stereochemistry of the components. Figures were prepared using UCSF chimera and PyMOL (Schrodinger, Inc).

### Example 20

Minivirus displaying SARS-CoV-2 Spike protein on its surface is blocked from binding its receptor ACE2 by linoleic acid. (A) Schematic illustration of a Minivirus with SARS-CoV-2 Spike protein on its surface, immobilized via their His-tag. (B) Representative confocal microscopy images of MCF7 human epithelial cells incubated for 10 min with Miniviruses, showing attachment of the Miniviruses to the cell's surface. Miniviruses are individually visualized as small dots. The inset shows magnified area of attachment. (C) Miniviruses allowed for a systematic assessment of changes in SARS-CoV-2 Spike protein-mediated cell interactions as a function of Free Fatty Acid (FFA) occupancy. Because recombinant native SARS-CoV-2 Spike protein binds the Free Fatty Acids Linoleic Acid and Oleic Acid during heterologous protein expression and purification, we first generated FFA-depleted Spike protein (ApoS) by treatment of the purified SARS-CoV-2 Spike protein with lipidex columns as described in Example 17. Compared to the native Spike protein, ApoS-decorated Miniviruses displayed increased binding to human epithelial cells (First two bars on the left of the chart). This is consistent with a model where FFAs lock a closed Spike protein conformation, inhibiting binding to its receptor, ACE2. Controlled loading of ApoS Miniviruses by incubating them with FFAs of differing length and saturation (i.e., palmitic acid (PA), oleic acid (OA), LA and arachidonic acid (AA)). Binding of OA, and LA, but not PA, in SARS-CoV-2 Spike protein was successfully verified by multiple reactions monitoring LC-MS/MS. Of note, addition of saturated PA did not significantly reduce Minivirus-ACE2 cell binding compared to ApoS. However, we found that the (poly)-unsaturated FFAs OA, LA and AA were able to reduce Minivirus binding compared to ApoS Miniviruses.

### Example 21

### Example 21 reveals via electron tomography the effect of linoleic acid on cultured mammalian cells infected with live SARS-CoV-2 virus.

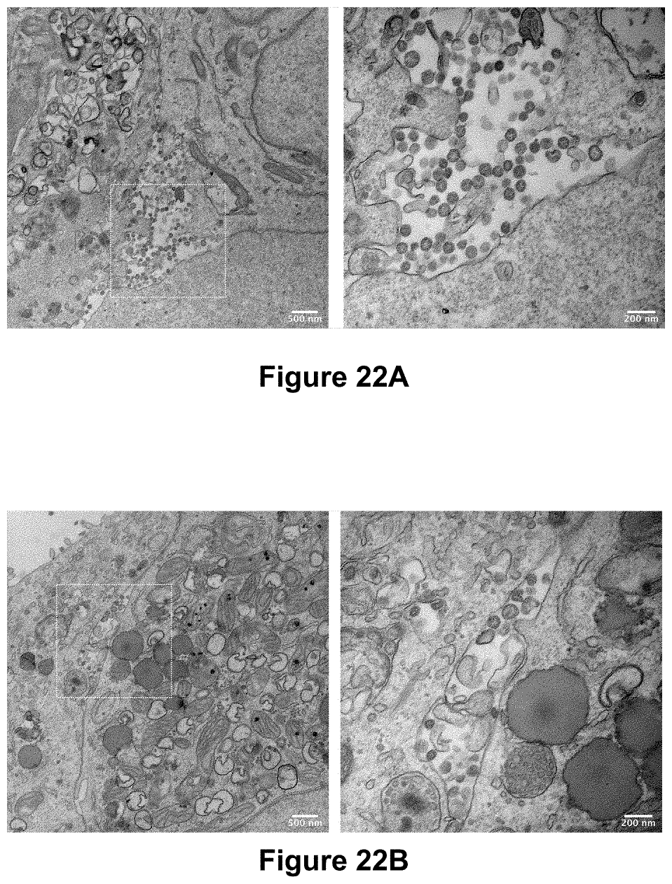

FIGS. 22A and 22B shows that the stereotypical enveloped mini-organelles known as “replication factories” inside SARS-CoV-2 are wrecked by the presence of linoleic acid, and the viral particles themselves also suffer visible deformation and destruction by the presence of linoleic acid.

The methods utilized to carry out the electron tomography are as follows:

Cells and virus propagation. A human gut epithelial cell line Caco2 expressing ACE2 (Caco-2-ACE2) (a kind gift of Dr Yohei Yamauchi, University of Bristol) were cultured at 37° C. in 5% CO2 in Dulbecco's modified Eagle's medium plus GlutaMAX (DMEM, Gibco, ThermoFisher) supplemented with 10% fetal bovine serum (FBS, Gibco, ThermoFisher) and 0.1 mM non-essential amino acids (NEAA, Sigma Aldrich). A SARS-CoV-2 reporter virus expressing a gene encoding the fluorescent protein turboGFP in place of the ORF7 gene (termed rSARS-CoV-2/Wuhan/ORF7-tGFP) was generated using a SARS-CoV-2 (Wuhan isolate) reverse genetics system by yeast-based transformation associated recombination cloning (manuscript in preparation). The virus was propagated in cells grown in infection medium (Eagle's minimum essential medium plus GlutaMAX (MEM, Gibco) supplemented with 2% FBS and NEAA). Cells were incubated at 37° C. in 5% CO2 until cytopathic effects were observed at which time the supernatant was harvested and filtered through a 0.2 um filter.

Viral detection by fluorescence. Caco-2-ACE2 cells were seeded onto glass coverslips coated with poly-D-lysine in 24 well plates or in μClear 96-well Microplates (Greiner Bio-one) in DMEM supplemented with 10% FBS until cell coverage on the coverslips reached 25%. The cells were inoculated with rSARS-CoV-2/Wuhan/ORF7-tGFP at MOI 5 in MEM supplemented with 2% FBS for 60 minutes at room temperature before the media was removed and replaced with infection medium containing 50 μM linoleic acid and 0.25% DMSO, or 0.25% DMSO only. Control wells were treated the same but received no infectious inoculum. Cells were incubated at 37° C. in 5% CO2 for 36 hours until turboGFP expression was detectable in cells in the 96 well plate by fluorescence imaging with an ImageXpress Pico Automated Cell Imaging System (Molecular Devices). Samples were inactivated and fixed by submersion in 4% paraformaldehyde (PFA) for 60 minutes at room temperature. All work with infectious recombinant SARS-CoV-2 was done inside a class III microbiological safety cabinet in a containment level 3 facility at the University of Bristol, UK.

Electron tomography. 300 nm sections collected on Pioloform-coated slot grids (Agar Scientific) were incubated in a solution of 15 nm gold fiducial markers (Aurion) for 5 min on each side. Tilt series (−65° to +65° at 1.5° increments) were acquired at 19,000× magnification (0.5261 nm/px) using a FEI Tecnai 20 transmission electron microscope operated at 200 kV and equipped with a 4k by 4k FEI Eagle camera. Electron tomograms were reconstructed using fiducial markers for alignment in IMOD.

Listing of SEQ ID NOs:

SARS-CoV-2 Spike protein used for
structural studies
SEQ ID NO: 1
MFVFLVLLPLVSSQCVNLTTRTQLPPAYTNSFTRGVYYPD
KVFRSSVLHSTQDLFLPFFSNVTWFHAIHVSGTNGTKRFD
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGFNCYFPLQSYGFQPTNGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PASVASQSIIAYTMSLGAENSVAYSNNSIAIPTNFTISVT
TEILPVSMTKTSVDCTMYICGDSTECSNLLLQYGSFCTQL
NRALTGIAVEQDKNTQEVFAQVKQIYKTPPIKDFGGFNFS
QILPDPSKPSKRSFIEDLLFNKVTLADAGFIKQYGDCLGD
IAARDLICAQKFNGLTVLPPLLTDEMIAQYTSALLAGTIT
SGWTFGAGAALQIPFAMQMAYRFNGIGVTQNVLYENQKLI
ANQFNSAIGKIQDSLSSTASALGKLQDVVNQNAQALNTLV
KQLSSNFGAISSVLNDILSRLDKVEAEVQIDRLITGRLQS
LQTYVTQQLIRAAEIRASANLAATKMSECVLGQSKRVDFC
GKGYHLMSFPQSAPHGVVFLHVTYVPAQEKNFTTAPAICH
DGKAHFPREGVFVSNGTHWFVTQRNFYEPQIITTDNTFVS
GNCDVVIGIVNNTVYDPLQPELDSFKEELDKYFKNHTSPD
VDLGDISGINASVVNIQKEIDRLNEVAKNLNESLIDLQEL
GKYEQYIKWPSGRLVPRGSPGSGYIPEAPRDGQAYVRKDG
EWVLLSTFLGHHHHHH
SARS-CoV-2 Spike protein full-length
SEQ ID NO: 2
MFVFLVLLPLVSSQCVNLTTRTQLPPAYTNSFTRGVYYPD
KVFRSSVLHSTQDLFLPFFSNVTWFHAIHVSGTNGTKRFD
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGFNCYFPLQSYGFQPTNGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPTNFTI
SVTTEILPVSMTKTSVDCTMYICGDSTECSNLLLQYGSFC
TQLNRALTGIAVEQDKNTQEVFAQVKQIYKTPPIKDFGGF
NFSQILPDPSKPSKRSFIEDLLFNKVTLADAGFIKQYGDC
LGDIAARDLICAQKFNGLTVLPPLLTDEMIAQYTSALLAG
TITSGWTFGAGAALQIPFAMQMAYRFNGIGVTQNVLYENQ
KLIANQFNSAIGKIQDSLSSTASALGKLQDVVNQNAQALN
TLVKQLSSNFGAISSVLNDILSRLDKVEAEVQIDRLITGR
LQSLQTYVTQQLIRAAEIRASANLAATKMSECVLGQSKRV
DFCGKGYHLMSFPQSAPHGVVFLHVTYVPAQEKNFTTAPA
ICHDGKAHFPREGVFVSNGTHWFVTQRNFYEPQIITTDNT
FVSGNCDVVIGIVNNTVYDPLQPELDSFKEELDKYFKNHT
SPDVDLGDISGINASVVNIQKEIDRLNEVAKNLNESLIDL
QELGKYEQYIKWPWYIWLGFIAGLIAIVMVTIMLCCMTSC
CSCLKGCCSCGSCCKFDEDDSEPVLKGVKLHYT
SARS-CoV Spike protein full-length
SEQ ID NO: 3
MFIFLLFLTLTSGSDLDRCTTFDDVQAPNYTQHTSSMRGV
YYPDEIFRSDTLYLTQDLFLPFYSNVTGFHTINHTFGNPV
IPFKDGIYFAATEKSNVVRGWVFGSTMNNKSQSVIIINNS
TNVVIRACNFELCDNPFFAVSKPMGTQTHTMIFDNAFNCT
FEYISDAFSLDVSEKSGNFKHLREFVFKNKDGFLYVYKGY
QPIDVVRDLPSGFNTLKPIFKLPLGINITNFRAILTAFSP
AQDIWGTSAAAYFVGYLKPTTFMLKYDENGTITDAVDCSQ
NPLAELKCSVKSFEIDKGIYQTSNFRVVPSGDVVRFPNIT
NLCPFGEVFNATKFPSVYAWERKKISNCVADYSVLYNSTF
FSTFKCYGVSATKLNDLCFSNVYADSFVVKGDDVRQIAPG
QTGVIADYNYKLPDDFMGCVLAWNTRNIDATSTGNYNYKY
RYLRHGKLRPFERDISNVPFSPDGKPCTPPALNCYWPLND
YGFYTTTGIGYQPYRVVVLSFELLNAPATVCGPKLSTDLI
KNQCVNFNFNGLTGTGVLTPSSKRFQPFQQFGRDVSDFTD
SVRDPKTSEILDISPCSFGGVSVITPGTNASSEVAVLYQD
VNCTDVSTAIHADQLTPAWRIYSTGNNVFQTQAGCLIGAE
HVDTSYECDIPIGAGICASYHTVSLLRSTSQKSIVAYTMS
LGADSSIAYSNNTIAIPTNFSISITTEVMPVSMAKTSVDC
NMYICGDSTECANLLLQYGSFCTQLNRALSGIAAEQDRNT
REVFAQVKQMYKTPTLKYFGGFNFSQILPDPLKPTKRSFI
EDLLFNKVTLADAGFMKQYGECLGDINARDLICAQKFNGL
TVLPPLLTDDMIAAYTAALVSGTATAGWTFGAGAALQIPF
AMQMAYRFNGIGVTQNVLYENQKQIANQFNKAISQIQESL
TTTSTALGKLQDVVNQNAQALNTLVKQLSSNFGAISSVLN
DILSRLDKVEAEVQIDRLITGRLQSLQTYVTQQLIRAAEI
RASANLAATKMSECVLGQSKRVDFCGKGYHLMSFPQAAPH
GVVFLHVTYVPSQERNFTTAPAICHEGKAYFPREGVFVFN
GTSWFITQRNFFSPQIITTDNTFVSGNCDVVIGIINNTVY
DPLQPELDSFKEELDKYFKNHTSPDVDLGDISGINASVVN
IQKEIDRLNEVAKNLNESLIDLQELGKYEQYIKWPWYVWL
GFIAGLIAIVMVTILLCCMTSCCSCLKGACSCGSCCKFDE
DDSEPVLKGVKLHYT
MERS-CoV Spike protein full-length
SEQ ID NO: 4
MIHSVFLLMFLLTPTESYVDVGPDSVKSACIEVDIQQTFF
DKTWPRPIDVSKADGIIYPQGRTYSNITITYQGLFPYQGD
HGDMYVYSAGHATGTTPQKLFVANYSQDVKQFANGFVVRI
GAAANSTGTVIISPSTSATIRKIYPAFMLGSSVGNFSDGK
MGRFFNHTLVLLPDGCGTLLRAFYCILEPRSGNHCPAGNS
YTSFATYHTPATDCSDGNYNRNASLNSFKEYFNLRNCTFM
YTYNITEDEILEWFGITQTAQGVHLFSSRYVDLYGGNMFQ
FATLPVYDTIKYYSIIPHSIRSIQSDRKAWAAFYVYKLQP
LTFLLDFSVDGYIRRAIDCGFNDLSQLHCSYESFDVESGV
YSVSSFEAKPSGSVVEQAEGVECDFSPLLSGTPPQVYNFK
RLVFTNCNYNLTKLLSLFSVNDFTCSQISPAAIASNCYSS
LILDYFSYPLSMKSDLSVSSAGPISQFNYKQSFSNPTCLI
LATVPHNLTTITKPLKYSYINKCSRLLSDDRTEVPQLVNA
NQYSPCVSIVPSTVWEDGDYYRKQLSPLEGGGWLVASGST
VAMTEQLQMGFGITVQYGTDTNSVCPKLEFANDTKIASQL
GNCVEYSLYGVSGRGVFQNCTAVGVRQQRFVYDAYQNLVG
YYSDDGNYYCLRACVSVPVSVIYDKETKTHATLFGSVACE
HISSTMSQYSRSTRSMLKRRDSTYGPLQTPVGCVLGLVNS
SLFVEDCKLPLGQSLCALPDTPSTLTPRSVRSVPGEMRLA
SIAFNHPIQVDQLNSSYFKLSIPTNFSFGVTQEYIQTTIQ
KVTVDCKQYVCNGFQKCEQLLREYGQFCSKINQALHGANL
RQDDSVRNLFASVKSSQSSPIIPGFGGDFNLTLLEPVSIS
TGSRSARSAIEDLLFDKVTIADPGYMQGYDDCMQQGPASA
RDLICAQYVAGYKVLPPLMDVNMEAAYTSSLLGSIAGVGW
TAGLSSFAAIPFAQSIFYRLNGVGITQQVLSENQKLIANK
FNQALGAMQTGFTTTNEAFRKVQDAVNNNAQALSKLASEL
SNTFGAISASIGDIIQRLDVLEQDAQIDRLINGRLTTLNA
FVAQQLVRSESAALSAQLAKDKVNECVKAQSKRSGFCGQG
THIVSFVVNAPNGLYFMHVGYYPSNHIEVVSAYGLCDAAN
PTNCIAPVNGYFIKTNNTRIVDEWSYTGSSFYAPEPITSL
NTKYVAPQVTYQNISTNLPPPLLGNSTGIDFQDELDEFFK
NVSTSIPNFGSLTQINTTLLDLTYEMLSLQQVVKALNESY
IDLKELGNYTYYNKWPWYIWLGFIAGLVALALCVFFILCC
TGCGTNCMGKLKCNRCCDRYEEYDLEPHKVHVH
HCoV-OC43 Spike protein full-length
SEQ ID NO: 5
MFLILLISLPTAFAVIGDLKCPLDTSYKGTFNNKDTGPPF
ISTDTVDVTNGLGTYYVLDRVYLNTTLFLNGYYPTSGSTY
RNMALKGTDKLSTLWFKPPFLSDFINGIFAKVKNTKVFKD
GVMYSEFPAITIGSTFVNTSYSVVVQPRTINSTQDGDNKL
QGLLEVSVCQYNMCEYPHTSCHPKLGNHFKELWHLDTGVV
SCLYKRNFTYDVNANYLYFHFYQEGGTFYAYFTDTGVVTK
FLFNVYLGMALSHYYVMPLTCISRRDIGFTLEYWVTPLTS
RQYLLAFNQDGIIFNAVDCMSDFMSEIKCKTQSIAPPTGV
YELNGYTVQPIADVYRRKPDLPNCNIEAWLNDKSVPSPLN
WERKTFSNCNFNMSSLMSFIQADSFTCNNIDAAKIYGMCF
SSITIDKFAIPNGRKVDLQLGNLGYLQSSNYRIDTTATSC
QLYYNLPAANVSVSRFNPSTWNKRFGFIEDSVFVPQPTGV
FTNHSVVYAQHCFKAPKNFCPCKLNGSCPGKNNGIGTCPA
GTNYLTCDNLCTLDPITFKAPGTYKCPQTKSLVGIGEHCS
GLAVKSDYCRGNSCTCQPQAFLGWSADSCLQGDKCNIFAN
LILHDVNSGLTCSTDLQKANTDIILGVCVNYDLYGISGQG
IFVEVNATYYNSWQNLLYDSNGNLYGFRDYITNRTFMIRS
CYSGRVSAAFHANSSEPALLFRNIKCNYVFNNSLTRQLQP
INYSFDSYLGCWNAYNSTAISVQTCDLTVGSGYCVDYSKN
RRSRRAITTGYRFTNFEPFTVNSVNDSLEPVGGLYEIQIP
SEFTIGNMEEFIQTSSPKVTIDCAAFVCGDYAACKLQLVE
YGSFCDNINAILTEVNELLDTTQLQVANSLMNGVTLSTKL
KDGVNFNVDDINFSPVLGCLGSECSKASSRSAIEDLLFDK
VKLSDVGFVEAYNNCTGGAEIRDLICVQSYKGIKVLPPLL
SENQISGYTLAATSASLFPPWTAAAGVPFYLNVQYRINGL
GVTMDVLSQNQKLIANAFNNALYAIQQGFDATNSALVKIQ
AVVNANAEALNNLLQQLSNRFGAISASLQEILSRLDALEA
EAQIDRLINGRLTALNAYVSQQLSDSTLVKFSAAQAMEKV
NECVKSQSSRINFCGNGNHIISLVQNAPYGLYFIHFNYVP
TKYVTAKVSPGLCIAGNRGIAPKSGYFVNVNNTWMYTGSG
YYYPEPITENNVVVMSTCAVNYTKAPYVMLNTSIPNLPDF
KEELDQWFKNQTSVAPDLSLDYINVTFLDLQVEMNRLQEA
IKVLNHSYINLKDIGTYEYYVKWPWYVWLLICLAGVAMLV
LLFFICCCTGCGTSCFKKCGGCCDDYTGYQELVIKTSHDD
HCoV-HKU1 Spike protein full-length
SEQ ID NO: 6
MLLIIFILPTTLAVIGDFNCTNFAINDLNTTVPRISEYVV
DVSYGLGTYYILDRVYLNTTILFTGYFPKSGANFRDLSLK
GTTYLSTLWYQKPFLSDFNNGIFSRVKNTKLYVNKTLYSE
FSTIVIGSVFINNSYTIVVQPHNGVLEITACQYTMCEYPH
TICKSKGSSRNESWHFDKSEPLCLFKKNFTYNVSTDWLYF
HFYQERGTFYAYYADSGMPTTFLFSLYLGTLLSHYYVLPL
TCNAISSNTDNETLQYWVTPLSKRQYLLKFDNRGVITNAV
DCSSSFFSEIQCKTKSLLPNTGVYDLSGFTVKPVATVHRR
IPDLPDCDIDKWLNNFNVPSPLNWERKIFSNCNFNLSTLL
RLVHTDSFSCNNFDESKIYGSCFKSIVLDKFAIPNSRRSD
LQLGSSGFLQSSNYKIDTTSSSCQLYYSLPAINVTINNYN
PSSWNRRYGFNNFNLSSHSVVYSRYCFSVNNTFCPCAKPS
FASSCKSHKPPSASCPIGTNYRSCESTTVLDHTDWCRCSC
LPDPITAYDPRSCSQKKSLVGVGEHCAGFGVDEEKCGVLD
GSYNVSCLCSTDAFLGWSYDTCVSNNRCNIFSNFILNGIN
SGTTCSNDLLQPNTEVFTDVCVDYDLYGITGQGIFKEVSA
VYYNSWQNLLYDSNGNIIGFKDFVTNKTYNIFPCYAGRVS
AAFHQNASSLALLYRNLKCSYVLNNISLTTQPYFDSYLGC
VFNADNLTDYSVSSCALRMGSGFCVDYNSPSSSSSRRKRR
SISASYRFVTFEPFNVSFVNDSIESVGGLYEIKIPTNFTI
VGQEEFIQTNSPKVTIDCSLFVCSNYAACHDLLSEYGTFC
DNINSILDEVNGLLDTTQLHVADTLMQGVTLSSNLNTNLH
FDVDNINFKSLVGCLGPHCGSSSRSFFEDLLFDKVKLSDV
GFVEAYNNCTGGSEIRDLLCVQSFNGIKVLPPILSESQIS
GYTTAATVAAMFPPWSAAAGIPFSLNVQYRINGLGVTMDV
LNKNQKLIATAFNNALLSIQNGFSATNSALAKIQSVVNSN
AQALNSLLQQLFNKFGAISSSLQEILSRLDALEAQVQIDR
LINGRLTALNAYVSQQLSDISLVKFGAALAMEKVNECVKS
QSPRINFCGNGNHILSLVQNAPYGLLFMHFSYKPISFKTV
LVSPGLCISGDVGIAPKQGYFIKHNDHWMFTGSSYYYPEP
ISDKNVVFMNTCSVNFTKAPLVYLNHSVPKLSDFESELSH
WFKNQTSIAPNLTLNLHTINATFLDLYYEMNLIQESIKSL
NNSYINLKDIGTYEMYVKWPWYVWLLISFSFIIFLVLLFF
ICCCTGCGSACFSKCHNCCDEYGGHHDFVIKTSHDD
HCoV-229E Spike protein full-length
SEQ ID NO: 7
MFVLLVAYALLHIAGCQTTNGLNTSYSVCNGCVGYSENVF
AVESGGYIPSDFAFNNWFLLTNTSSVVDGVVRSFQPLLLN
CLWSVSGLRFTTGFVYFNGTGRGDCKGFSSDVLSDVIRYN
LNFEENLRRGTILFKTSYGVVVFYCTNNTLVSGDAHIPFG
TVLGNFYCFVNTTIGNETTSAFVGALPKTVREFVISRTGH
FYINGYRYFTLGNVEAVNFNVTTAETTDFCTVALASYADV
LVNVSQTSIANIIYCNSVINRLRCDQLSFDVPDGFYSTSP
IQSVELPVSIVSLPVYHKHTFIVLYVDFKPQSGGGKCFNC
YPAGVNITLANFNETKGPLCVDTSHFTTKYVAVYANVGRW
SASINTGNCPFSFGKVNNFVKFGSVCFSLKDIPGGCAMPI
VANWAYSKYYTIGSLYVSWSDGDGITGVPQPVEGVSSFMN
VTLDKCTKYNIYDVSGVGVIRVSNDTFLNGITYTSTSGNL
LGFKDVTKGTIYSITPCNPPDQLVVYQQAVVGAMLSENFT
SYGFSNVVELPKFFYASNGTYNCTDAVLTYSSFGVCADGS
IIAVQPRNVSYDSVSAIVTANLSIPSNWTTSVQVEYLQIT
STPIVVDCSTYVCNGNVRCVELLKQYTSACKTIEDALRNS
ARLESADVSEMLTFDKKAFTLANVSSFGDYNLSSVIPSLP
TSGSRVAGRSAIEDILFSKLVTSGLGTVDADYKKCTKGLS
IADLACAQYYNGIMVLPGVADAERMAMYTGSLIGGIALGG
LTSAVSIPFSLAIQARLNYVALQTDVLQENQKILAASFNK
AMTNIVDAFTGVNDAITQTSQALQTVATALNKIQDVVNQQ
GNSLNHLTSQLRQNFQAISSSIQAIYDRLDTIQADQQVDR
LITGRLAALNVFVSHTLTKYTEVRASRQLAQQKVNECVKS
QSKRYGFCGNGTHIFSIVNAAPEGLVFLHTVLLPTQYKDV
EAWSGLCVDGTNGYVLRQPNLALYKEGNYYRITSRIMFEP
RIPTMADFVQIENCNVTFVNISRSELQTIVPEYIDVNKTL
QELSYKLPNYTVPDLVVEQYNQTILNLTSEISTLENKSAE
LNYTVQKLQTLIDNINSTLVDLKWLNRVETYIKWPWWVWL
CISVVLIFVVSMLLLCCCSTGCCGFFSCFASSIRGCCEST
KLPYYDVEKIHIQ
HCoV-NL63 Spike protein full-length
SEQ ID NO: 8
MKLFLILLVLPLASCFFTCNSNANLSMLQLGVPDNSSTIV
TGLLPTHWFCANQSTSVYSANGFFYIDVGNHRSAFALHTG
YYDANQYYIYVTNEIGLNASVTLKICKFSRNTTFDFLSNA
SSSFDCIVNLLFTEQLGAPLGITISGETVRLHLYNVTRTF
YVPAAYKLTKLSVKCYFNYSCVFSVVNATVTVNVTTHNGR
VVNYTVCDDCNGYTDNIFSVQQDGRIPNGFPFNNWFLLTN
GSTLVDGVSRLYQPLRLTCLWPVPGLKSSTGFVYFNATGS
DVNCNGYQHNSVVDVMRYNLNFSANSLDNLKSGVIVFKTL
QYDVLFYCSNSSSGVLDTTIPFGPSSQPYYCFINSTINTT
HVSTFVGILPPTVREIVVARTGQFYINGFKYFDLGFIEAV
NFNVTTASATDFWTVAFATFVDVLVNVSATNIQNLLYCDS
PFEKLQCEHLQFGLQDGFYSANFLDDNVLPETYVALPIYY
QHTDINFTATASFGGSCYVCKPHQVNISLNGNTSVCVRTS
HFSIRYIYNRVKSGSPGDSSWHIYLKSGTCPFSFSKLNNF
QKFKTICFSTVEVPGSCNFPLEATWHYTSYTIVGALYVTW
SEGNSITGVPYPVSGIREFSNLVLNNCTKYNIYDYVGTGI
IRSSNQSLAGGITYVSNSGNLLGFKNVSTGNIFIVTPCNQ
PDQVAVYQQSIIGAMTAVNESRYGLQNLLQLPNFYYVSNG
GNNCTTAVMTYSNFGICADGSLIPVRPRNSSDNGISAIIT
ANLSIPSNWTTSVQVEYLQITSTPIVVDCATYVCNGNPRC
KNLLKQYTSACKTIEDALRLSAHLETNDVSSMLTFDSNAF
SLANVTSFGDYNLSSVLPQRNIRSSRIAGRSALEDLLFSK
VVTSGLGTVDVDYKSCTKGLSIADLACAQYYNGIMVLPGV
ADAERMAMYTGSLIGGMVLGGLTSAAAIPFSLALQARLNY
VALQTDVLQENQKILAASFNKAINNIVASFSSVNDAITQT
AEAIHTVTIALNKIQDVVNQQGSALNHLTSQLRHNFQAIS
NSIQAIYDRLDSIQADQQVDRLITGRLAALNAFVSQVLNK
YTEVRGSRRLAQQKINECVKSQSNRYGFCGNGTHIFSIVN
SAPDGLLFLHTVLLPTDYKNVKAWSGICVDGIYGYVLRQP
NLVLYSDNGVFRVTSRVMFQPRLPVLSDFVQIYNCNVTFV
NISRVELHTVIPDYVDVNKTLQEFAQNLPKYVKPNFDLTP
FNLTYLNLSSELKQLEAKTASLFQTTVELQGLIDQINSTY
VDLKLLNRFENYIKWPWWVWLIISVVFVVLLSLLVFCCLS
TGCCGCCNCLTSSMRGCCDCGSTKLPYYEFEKVHVQ
SARS-CoV-2 Spike protein RBD domain
SEQ ID NO: 9
SETKCTLKSFTVEKGIYQTSNFRVQPTESIVRFPNITNLC
PFGEVFNATRFASVYAWNRKRISNCVADYSVLYNSASFST
FKCYGVSPTKLNDLCFTNVYADSFVIRGDEVRQI
SARS-CoV Spike protein RBD domain
SEQ ID NO: 10
AELKCSVKSFEIDKGIYQTSNFRVVPSGDVVRFPNITNLC
PFGEVFNATKFPSVYAWERKKISNCVADYSVLYNSTFFST
FKCYGVSATKLNDLCFSNVYADSFVVKGDDVRQI
MERS-CoV Spike protein RBD domain
SEQ ID NO: 11
SQLHCSYESFDVESGVYSVSSFEAKPSGSVVEQAEGVECD
FSPLLSGTPPQVYNFKRLVFTNCNYNLTKLLSLFSVNDFT
CSQISPAAIASNCYSSLILDYFSYPLSMKSDL
HCoV-OC43 Spike protein RBD domain
SEQ ID NO: 12
SEIKCKTQSIAPPTGVYELNGYTVQPIADVYRRKPDLPNC
NIEAWLNDKSVPSPLNWERKTFSNCNFNMSSLMSFIQADS
FTCNNIDAAKIYGMCFSSITIDKFAIPNGRKVDL
HCoV-HKU1 Spike protein RBD domain
SEQ ID NO: 13
SEIQCKTKSLLPNTGVYDLSGFTVKPVATVHRRIPDLPDC
DIDKWLNNFNVPSPLNWERKIFSNCNFNLSTLLRLVHTDS
FSCNNFDESKIYGSCFKSIVLDKFAIPNSRRSDL
HCoV-229E Spike protein RBD domain
SEQ ID NO: 14
NRLRCDQLSFDVPDGFYSTSPIQSVELPVSIVSLPVYHKH
TFIVLYVDFKPQSGGGKCFNCYPAGVNITLANFNETKGPL
HCoV-NL63 Spike protein RBD domain
SEQ ID NO: 15
EKLQCEHLQFGLQDGFYSANFLDDNVLPETYVALPIYYQH
TDINFTATASFGGSCYVCKPHQVNISLNGNTSV

### REFERENCES CITED

1 Varga, Z. et al. Endothelial cell infection and endotheliitis in COVID-19. Lancet 395, 1417-1418, doi :10.1016/SO140-6736(20)30937-5 (2020).
2 Tay, M. Z., Poh, C. M., Renia, L., MacAry, P. A. & Ng, L. F. P. The trinity of COVID-19: immunity, inflammation and intervention. Nat Rev Immunol 20, 363-374, doi:10.1038/541577-020-0311-8 (2020).
3 Zhou, P. et aL A pneumonia outbreak associated with a new coronavirus of probable bat origin. Nature 579, 270-273, doi:10.1038/s41586-020-2012-7 (2020).
4 Yan, B. et al. Characterization of the Lipidomic Profile of Human Coronavirus-Infected Cells: Implications for Lipid Metabolism Remodeling upon Coronavirus Replication. Viruses 11, doi:10.3390/v11010073 (2019).
5 Zumla, A., Chan, J. F., Azhar, E. I., Hui, D. S. & Yuen, K. Y. Coronaviruses—drug discovery and therapeutic options. Nat Rev Drug Discov 15, 327-347, doi:10.1038/nrd.2015.37 (2016).
6 Wu, J. T. et al. Estimating clinical severity of COVID-19 from the transmission dynamics in Wuhan, China. Nat Med 26, 506-510, doi:10.1038/541591-020-0822-7 (2020).
7 Grasselli, G. et al. Baseline Characteristics and Outcomes of 1591 Patients Infected With SARS-CoV-2 Admitted to ICUs of the Lombardy Region, Italy. JAMA, doi:10.1001/jama.2020.5394 (2020).
8 Cummings, M. J. et al. Epidemiology, clinical course, and outcomes of critically ill adults with COVID-19 in New York City: a prospective cohort study. Lancet, doi :10.1016/SO140-6736(20)31189-2 (2020).
9 Hoffmann, M. et al. SARS-CoV-2 Cell Entry Depends on ACE2 and TMPRSS2 and Is Blocked by a Clinically Proven Protease Inhibitor. Cell 181, 271-280 e278, doi:10.1016/j.ce11.2020.02.052 (2020).
10 Matsuyama, S. et al. Enhanced isolation of SARS-CoV-2 by TMPRSS2-expressing cells. Proc Natl Acad Sci USA 117, 7001-7003, doi:10.1073/pnas.2002589117 (2020).
11 Hoffmann, M., Kleine-Weber, H. & Pohlmann, S. A Multibasic Cleavage Site in the Spike Protein of SARS-CoV-2 Is Essential for Infection of Human Lung Cells. Mol Cell 78, 779-784 e775, doi:10.1016/j.molce1.2020.04.022 (2020).
12 Du, L. et al. The spike protein of SARS-CoV--a target for vaccine and therapeutic development. Nat Rev Microbiol 7, 226-236, doi:10.1038/nrmicro2090 (2009).
13 Letko, M., Marzi, A. & Munster, V. Functional assessment of cell entry and receptor usage for SARS-CoV-2 and other lineage B betacoronaviruses. Nat Microbiol 5, 562-569, doi:10.1038/s41564-020-0688-y (2020).
14 Walls, A. C. et al. Structure, Function, and Antigenicity of the SARS-CoV-2 Spike Glycoprotein. Cell 181, 281-292 e286, doi:10.1016/j.ce11.2020.02.058 (2020).
15 Kindler, E. & Thiel, V. To sense or not to sense viral RNA--essentials of coronavirus innate immune evasion. Curr Opin Microbiol 20, 69-75, doi:10.1016/j.mib.2014.05.005 (2014).
16 Kikkert, M. Innate Immune Evasion by Human Respiratory RNA Viruses. J Innate Immun 12, 4-20, doi:10.1159/000503030 (2020).
17 Zang, R. et al. TMPRSS2 and TMPRSS4 promote SARS-CoV-2 infection of human small intestinal enterocytes. Sci Immunol 5, doi:10.1126/sciimmunol.abc3582 (2020).
18 Qin, C. et al. Dysregulation of immune response in patients with COVID-19 in Wuhan, China. Clin Infect Dis, doi:10.1093/cid/ciaa248 (2020).
19 Ziegler, C. G. K. et al. SARS-CoV-2 Receptor ACE2 Is an Interferon-Stimulated Gene in Human Airway Epithelial Cells and Is Detected in Specific Cell Subsets across Tissues. Cell 181, 1016-1035 e1019, doi:10.1016/j.ce11.2020.04.035 (2020).
20 Shen, B. et al. Proteomic and Metabolomic Characterization of COVID-19 Patient Sera. Cell, doi:10.1016/j.ce11.2020.05.032 (2020).
21 Goodwin, C. M., Xu, S. & Munger, J. Stealing the Keys to the Kitchen: Viral Manipulation of the Host Cell Metabolic Network. Trends Microbiol 23, 789-798, doi:10.1016/j.tim.2015.08.007 (2015).
22 Schoggins, J. W. & Randall, G. Lipids in innate antiviral defense. Cell Host Microbe 14, 379-385, doi:10.1016/j.chom.2013.09.010 (2013).
23 Nagamine, T., Inaba, T. & Sako, Y. A nuclear envelop-associated baculovirus protein promotes intranuclear lipid accumulation during infection. Virology 532, 108-117, doi:10.1016/j.viro1.2019.04.006 (2019).
24 Zaman, M. M. et al. Linoleic acid supplementation results in increased arachidonic acid and eicosanoid production in CF airway cells and in cftr−/− transgenic mice. Am J Physiol Lung Cell Mol Physiol 299, L599-606, doi:10.115²/ₐjplung.00346.2009 (2010).
25 Richieri GV, Ogata RT, Kleinfeld AM. The measurement of free fatty acid concentration with the fluorescent probe ADIFAB: a practical guide for the use of the ADIFAB probe. Mol Cell Biochem. 1999;192(1-2):87-94.
26 Toelzer et al. Free fatty acid binding pocket in the locked structure of SARS-CoV-2 spike protein. Science. 370, 725-730 (2020).

1.-95. (canceled)

96. An isolated complex of a coronavirus spike protein or a fragment or mutant thereof wherein said fragment or mutant at least contains a receptor binding domain of said coronavirus spike protein, with linoleic acid, or a derivative, salt or mimetic thereof, wherein the derivative or mimetic of linoleic acid is a compound characterised by the following general formula (I): 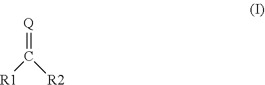
wherein Q is selected from O, S and NH;
R1 is selected from OH, NH₂, and SH; and
R2 is a straight hydrocarbyl group having from 13 to 21 C atoms or having 17 C atoms, optionally linked or bound to a detectable label;
wherein the mutant has at least 90%, or at least 95%, or has at least 96%, or at least 97%, or at least 98%, or at least 99% sequence homology with the wild-type coronavirus spike protein or fragment thereof; and
optionally, wherein the derivative or mimetic of the linoleic acid comprises a detectable label.

97. The complex of claim 96 wherein R2 has at least one unsaturated C—C bond, or two unsaturated C—C bonds, preferably the unsaturated C—C bonds are between C-8 and C-9 and between C-11 and C-12 of the hydrocarbyl group when counted from the carbon bound to the C=Q group in formula (I).

98. The complex of claim 96 wherein the compound is selected from the group consisting of oleic acid, arachidonic acid, elaidic acid, eicosapentaenoic acid, stearic acid, gamma-linoleic acid, calendic acid, arachidic acid, dihomo-gamma-linoleic acid, docosadienoic acid, adrenic acid, palmitic acid and behenic acid, preferably oleic acid.

99. The complex of claim 96 wherein the coronavirus spike protein or fragment or mutant thereof is selected from spike proteins or fragments or mutants thereof of a coronavirus causing respiratory disease, preferably pneumonia, preferably in humans.

100. The complex of claim 99 wherein the coronavirus spike protein or fragment thereof is a spike protein or fragment or mutant thereof of a coronavirus selected from the group consisting of SARS-CoV, MERS-CoV and SARS-CoV-2, preferably from SARS-CoV-2.

101. The complex of claim 96 wherein the coronavirus spike protein or fragment thereof has an amino acid sequence selected from the group consisting of the amino acids sequences according to SEQ ID NO: 1 to 15, preferably selected from SEQ ID NO. 1, 2, 3, 4, 9, 10 and 11.

102. The complex of claim 96 wherein the complex is immobilized, preferably on a surface of a test device.

103. The complex of claim 96 wherein the complex is bound to a receptor for the coronavirus spike protein or fragment or mutant thereof, preferably ACE2, wherein, optionally, the receptor is immobilized, preferably on a surface of a test device.

104. A method for producing a complex according to claim 96, the method being selected from the group consisting of:
(1) a method comprising the step of expressing the coronavirus spike protein or a fragment or mutant thereof of said coronavirus spike protein, wherein the fragment or mutant at least contains the receptor binding domain of said coronavirus spike protein, in a recombinant host cell in the presence of linoleic acid or a derivative or a salt or a mimetic thereof, and, optionally purifying the complex from the host cell;
(2) a method comprising the steps of:
(i) introducing into host cells a heterologous nucleic acid encoding the coronavirus spike protein or a fragment or mutant thereof wherein said fragment or mutant at least contains the receptor binding domain of said coronavirus spike protein;
(ii) culturing said host cells in the presence of linoleic acid or a derivative or a salt or a mimetic thereof; and, optionally
(iii) purifying the complex from the host cells and/or the culture medium;
(3) a method comprising the step of incubating an isolated coronavirus spike protein or fragment or mutant thereof wherein said fragment or mutant thereof at least contains a receptor binding domain of said coronavirus spike protein, with linoleic acid or a derivative or a salt or a mimetic thereof; and
(4) a method comprising the steps of:
(i) expressing the coronavirus spike protein or a fragment or mutant thereof wherein the fragment or mutant at least contains the receptor binding domain of said coronavirus spike protein in a recombinant host cell, preferably a linoleic acid-free host cell and medium;
(ii) isolating the expressed coronavirus spike protein or a fragment or mutant thereof; and
(iii) incubating the isolated coronavirus spike protein or fragment or mutant thereof with linoleic acid or a derivative or a salt or a mimetic thereof.

105. An in vitro assay for detecting whether a candidate molecule inhibits the binding of a complex according to claim 96 to a receptor protein for a coronavirus spike protein or fragment or mutant thereof, comprising the steps of:
(a) contacting the complex with a receptor protein for the coronavirus spike protein;
(b) contacting the complex and the receptor of (a) with the candidate molecule; and
(c) detecting unbound receptor and/or unbound coronavirus spike protein or mutant or fragment thereof.
wherein, optionally steps (a) and (b) are carried out simultaneously, or, optionally wherein step (b) is carried out before step (a)

106. An in vitro assay for detecting whether a candidate molecule inhibits the binding of a linoleic acid or derivative or salt or mimetic thereof to a coronavirus spike protein or fragment or mutant thereof wherein the fragment or mutant thereof at least contains the receptor binding domain of said coronavirus spike protein, comprising the steps of:
(A) contacting a coronavirus spike protein or fragment or mutant thereof according to claim 96 with the candidate molecule and the linoleic acid or derivative or salt or mimetic thereof; and
(B) measuring the amount of
(B1) the candidate molecule bound to the coronavirus spike protein or fragment or mutant thereof; and/ or
(B2) linoleic acid or derivative or salt or mimetic unbound to the coronavirus spike protein or fragment or mutant thereof.

107. The assay of claim 106, further comprising the step of determining a Kd value of the candidate molecule to the coronavirus spike protein or fragment or mutant thereof.

108. The assay of claim 107, further comprising the steps of:
(I) performing the method with multiple different candidate molecules wherein the method is carried out for each candidate molecule in a single reaction;
(II) determining a Kd value for each candidate molecule;
(III) selecting a candidate molecule having a predetermined threshold Kd value to the coronavirus spike protein or mutant or fragment thereof.

109. The assay of claim 108 wherein the threshold Kd value is below 100 nM.

110. A method for the treatment and/or prevention of a coronavirus infection by administration of a composition comprising linoleic acid, or a derivative or salt or mimetic thereof, in form of an aerosol formulation or dry powder formulation to the respiratory tract of a subject, wherein the derivative, salt of mimetic binds to the coronavirus spike protein of said coronavirus and wherein the linoleic acid or derivative or salt of mimetic thereof is used in a non-vesicular form, the derivative, salt or mimetic being a compound characterised by the following general formula (I): 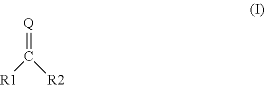
wherein
Q is selected from O, S and NH;
R1 is selected from OH, NH2, and SH; and
R2 is a straight hydrocarbyl group having from 13 to 21 C atoms, preferably 17 C atoms.

111. The method of claim 110, wherein the composition is administered by nasal administration in a unit dose of 1 to 84 μg, preferably 20 μg, of said linoleic acid or derivative or salt or mimetic thereof, and/or
wherein the composition is administered to the lower respiratory tract in a unit dose of 1 to 500 pg, preferably 20 pg, of said linoleic acid or derivative or salt or mimetic thereof.

112. The method of claim 110, wherein R2 has at least one unsaturated C—C bond, preferably two unsaturated C—C bonds, preferably the unsaturated C—C bonds are between C-8 and C-9 and between C-11 and C-12 of the hydrocarbyl group when counted from the carbon bound to the C=Q group in formula (I).

113. The method of claim 110, wherein the compound of formula (I) is selected from the group consisting of linoleic acid, oleic acid, arachidonic acid, elaidic acid, eicosapentaenoic acid, stearic acid, gamma-linoleic acid, calendic acid, arachidic acid, dihomo-gamma-linoleic acid, docosadienoic acid, adrenic acid, palm itic acid and behenic acid, preferably oleic acid.

114. The method of claim 110, wherein the linoleic acid or derivative or salt thereof is contained in a monophasic, preferably aqueous, solution or in a dry powder.

115. The method of claim 110 wherein the solution contains a fatty acid solubilizer, preferably selected from the group consisting of cyclodextrin, ethanol, propylene glycol and a polypropylene glycol, and mixtures of two or more thereof.

116. The method of claim 115 wherein the cyclodextrin is a β-cyclodextrin, preferably selected from the group consisting of O-methylated, acetylated, hydroxypropylated, hydroxyethylated, hydroxyisobutylated, glucosylated, maltosylated and sulfoalkylether-β-cyclodextrin and mixtures of two or more thereof.

117. The method of claim 116 wherein the molar ratio between the cyclodextrin and the linoleic acid or derivative or salt or mimetic thereof is at least 10 to 1, preferably 10:1 to 60:1.

118. The method of claim 110 wherein the coronavirus is a coronavirus causing respiratory disease in humans, inckusing but not limited to SARS-CoV, MERS-CoV and SARS-CoV-2.

119. The method of claim 110 wherein the aerosol formulation or dry powder formulation is administered by the use of a respiratory delivery device, preferably selected from the group consisting of a nebulizer, vaporizer, vapor inhaler, squeeze bottle, metered-dose spray pump, Bi-dir Multi-dose spray pump, a gas driven spray system/atomizer, electrically powered Nebulizers/Atomizers, mechanical powder sprayers, breath actuated inhaler, insufflator, meter dose inhaler, and a dry powder inhaler.

120. A method for selecting binder molecules, binding to the complex according to claim 96, from a library of multiple candidate binder molecules comprising the steps of:
(α) contacting the complex with the library of multiple candidate binder molecules; and
(β) detecting which of the multiple candidate binder molecules have bound to the complex.

121. Use of the complex according to claim 96 for the production of antibodies wherein a non-human animal is immunized with said complex.

122. A method for producing antibodies binding to a complex according to claim 96 comprising the steps of
immunizing a non-human animal with said complex; and
isolating antibodies binding to said complex form said animal.

123. A respiratory delivery device comprising a composition in the form of an aerosol formulation or a dry powder formulation containing linoleic acid, or a derivative or salt or mimetic thereof, in non-vesicular form and a fatty acid solubilizer wherein the linoleic acid, or a derivative or salt or mimetic binds to the spike protein of a coronavirus, the derivative, salt or mimetic being a compound characterised by the following general formula (I): 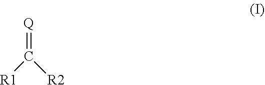
wherein
Q is selected from O, S and NH;
R1 is selected from OH, NH₂, and SH; and
R2 is a straight hydrocarbyl group having from 13 to 21 C atoms, preferably 17 C atoms.

124. The delivery device according to claim 123 wherein the composition further comprises one or more further ingredients selected from a mucoadhesive, at least one anti-oxidant and at least one propellant.

125. The delivery device of claim 124 wherein the mucoadhesive is selected from the group consisting of cellulose and derivatives thereof, more preferably methylcellulose, hydroxypropyl methylcellulose, microcrystalline cellulose, carboxymethylcellulose, hydroxyethyl cellulose or a mixture of two or more thereof; and/or
wherein the antioxidant is selected from the group consisting of ascorbic acid, tocopherols, EDTA, butylated hydroxytoluene (BHT), butylated hydroxyanisole (BHA), propyl gallate, ascorbyl fatty acid esters and mixtures of two or more thereof; and/or
wherein the propellant is a hydrofluoroalkane, preferably selected from the group consisting of hydrofluoroalkane, more preferably HFA 227, HFA 134a or a mixture thereof.

126. The delivery device according to claim 123, selected from the group consisting of a nebulizer, vaporizer, vapor inhaler, squeeze bottle, metered-dose spray pump, Bi-dir Multi-dose spray pump (OptiNose), a gas driven spray system/atomizer, electrically powered Nebulizers/Atomizers, mechanical powder sprayers, breath actuated inhaler, insufflator, meter dose inhaler, and a dry powder inhaler.

127. The delivery device of claim 123 wherein the device is adapted for intra-nasal delivery of said composition in a unit dose of 1 to 84 μg, preferably 20 μg, of said linoleic acid or derivative or salt or mimetic thereof; and/or wherein the device is adapted for pulmonary delivery of said composition in a unit dose of 1 to 500 μg, preferably 20 μg, of said linoleic acid or derivative or salt or mimetic thereof.

128. The delivery device of claim 123 wherein R2 has at least one unsaturated C—C bond, or two unsaturated C—C bonds, preferably the unsaturated C—C bonds are between C-8 and C-9 and between C-11 and C-12 of the hydrocarbyl group when counted from the carbon bound to the C=Q group in formula (I).

129. The delivery device of claim 123 wherein the compound is selected from the group consisting of oleic acid, arachidonic acid, elaidic acid, eicosapentaenoic acid, stearic acid, gamma-linoleic acid, calendic acid, arachidic acid, dihomo-gamma-linoleic acid, docosadienoic acid, adrenic acid, palm itic acid and behenic acid, preferably oleic acid.

130. The delivery device of claim 123 wherein the composition is a monophasic aqueous solution.

131. The delivery device of claim 123 wherein the composition includes a fatty acid stabilizer.


SEQUENCE LISTING (18 sequences)

SEQ ID NO: 1 - protein, 1256 aa, Artificial Sequence
  SARS-CoV-2 Spike protein used for structural studies
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRLVPRGSPGSGYIPEAPRDGQAYVRKDGEWVLLSTFLGHHHHHH

SEQ ID NO: 2 - protein, 1273 aa, SARS coronavirus
  SARS-CoV-2 Spike protein full-length
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1255 aa, SARS coronavirus
  SARS-CoV Spike protein full-length
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 4 - protein, 1353 aa, Betacoronavirus
  MERS-CoV Spike protein full-length
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 5 - protein, 1361 aa, Betacoronavirus
  HCoV-OC43 Spike protein full-length
MFLILLISLPTAFAVIGDLKCPLDTSYKGTFNNKDTGPPFISTDTVDVTNGLGTYYVLDRVYLNTTLFLNGYYPTSGSTYRNMALKGTDKLSTLWFKPPFLSDFINGIFAKVKNTKVFKDGVMYSEFPAITIGSTFVNTSYSVVVQPRTINSTQDGDNKLQGLLEVSVCQYNMCEYPHTSCHPKLGNHFKELWHLDTGVVSCLYKRNFTYDVNANYLYFHFYQEGGTFYAYFTDTGVVTKFLFNVYLGMALSHYYVMPLTCISRRDIGFTLEYWVTPLTSRQYLLAFNQDGIIFNAVDCMSDFMSEIKCKTQSIAPPTGVYELNGYTVQPIADVYRRKPDLPNCNIEAWLNDKSVPSPLNWERKTFSNCNFNMSSLMSFIQADSFTCNNIDAAKIYGMCFSSITIDKFAIPNGRKVDLQLGNLGYLQSSNYRIDTTATSCQLYYNLPAANVSVSRFNPSTWNKRFGFIEDSVFVPQPTGVFTNHSVVYAQHCFKAPKNFCPCKLNGSCPGKNNGIGTCPAGTNYLTCDNLCTLDPITFKAPGTYKCPQTKSLVGIGEHCSGLAVKSDYCRGNSCTCQPQAFLGWSADSCLQGDKCNIFANLILHDVNSGLTCSTDLQKANTDIILGVCVNYDLYGISGQGIFVEVNATYYNSWQNLLYDSNGNLYGFRDYITNRTFMIRSCYSGRVSAAFHANSSEPALLFRNIKCNYVFNNSLTRQLQPINYSFDSYLGCVVNAYNSTAISVQTCDLTVGSGYCVDYSKNRRSRRAITTGYRFTNFEPFTVNSVNDSLEPVGGLYEIQIPSEFTIGNMEEFIQTSSPKVTIDCAAFVCGDYAACKLQLVEYGSFCDNINAILTEVNELLDTTQLQVANSLMNGVTLSTKLKDGVNFNVDDINFSPVLGCLGSECSKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALYAIQQGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFNYVPTKYVTAKVSPGLCIAGNRGIAPKSGYFVNVNNTWMYTGSGYYYPEPITENNVVVMSTCAVNYTKAPYVMLNTSIPNLPDFKEELDQWFKNQTSVAPDLSLDYINVTFLDLQVEMNRLQEAIKVLNHSYINLKDIGTYEYYVKWPWYVWLLICLAGVAMLVLLFFICCCTGCGTSCFKKCGGCCDDYTGYQELVIKTSHDD

SEQ ID NO: 6 - protein, 1356 aa, Betacoronavirus
  HCoV-HKU1 Spike protein full-length
MLLIIFILPTTLAVIGDFNCTNFAINDLNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDWLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVFTDVCVDYDLYGITGQGIFKEVSAVYYNSWQNLLYDSNGNIIGFKDFVTNKTYNIFPCYAGRVSAAFHQNASSLALLYRNLKCSYVLNNISLTTQPYFDSYLGCVFNADNLTDYSVSSCALRMGSGFCVDYNSPSSSSSRRKRRSISASYRFVTFEPFNVSFVNDSIESVGGLYEIKIPTNFTIVGQEEFIQTNSPKVTIDCSLFVCSNYAACHDLLSEYGTFCDNINSILDEVNGLLDTTQLHVADTLMQGVTLSSNLNTNLHFDVDNINFKSLVGCLGPHCGSSSRSFFEDLLFDKVKLSDVGFVEAYNNCTGGSEIRDLLCVQSFNGIKVLPPILSESQISGYTTAATVAAMFPPWSAAAGIPFSLNVQYRINGLGVTMDVLNKNQKLIATAFNNALLSIQNGFSATNSALAKIQSVVNSNAQALNSLLQQLFNKFGAISSSLQEILSRLDALEAQVQIDRLINGRLTALNAYVSQQLSDISLVKFGAALAMEKVNECVKSQSPRINFCGNGNHILSLVQNAPYGLLFMHFSYKPISFKTVLVSPGLCISGDVGIAPKQGYFIKHNDHWMFTGSSYYYPEPISDKNVVFMNTCSVNFTKAPLVYLNHSVPKLSDFESELSHWFKNQTSIAPNLTLNLHTINATFLDLYYEMNLIQESIKSLNNSYINLKDIGTYEMYVKWPWYVWLLISFSFIIFLVLLFFICCCTGCGSACFSKCHNCCDEYGGHHDFVIKTSHDD

SEQ ID NO: 7 - protein, 1173 aa, Betacoronavirus
  HCoV-229E Spike protein full-length
MFVLLVAYALLHIAGCQTTNGLNTSYSVCNGCVGYSENVFAVESGGYIPSDFAFNNWFLLTNTSSVVDGVVRSFQPLLLNCLWSVSGLRFTTGFVYFNGTGRGDCKGFSSDVLSDVIRYNLNFEENLRRGTILFKTSYGVVVFYCTNNTLVSGDAHIPFGTVLGNFYCFVNTTIGNETTSAFVGALPKTVREFVISRTGHFYINGYRYFTLGNVEAVNFNVTTAETTDFCTVALASYADVLVNVSQTSIANIIYCNSVINRLRCDQLSFDVPDGFYSTSPIQSVELPVSIVSLPVYHKHTFIVLYVDFKPQSGGGKCFNCYPAGVNITLANFNETKGPLCVDTSHFTTKYVAVYANVGRWSASINTGNCPFSFGKVNNFVKFGSVCFSLKDIPGGCAMPIVANWAYSKYYTIGSLYVSWSDGDGITGVPQPVEGVSSFMNVTLDKCTKYNIYDVSGVGVIRVSNDTFLNGITYTSTSGNLLGFKDVTKGTIYSITPCNPPDQLVVYQQAVVGAMLSENFTSYGFSNVVELPKFFYASNGTYNCTDAVLTYSSFGVCADGSIIAVQPRNVSYDSVSAIVTANLSIPSNWTTSVQVEYLQITSTPIVVDCSTYVCNGNVRCVELLKQYTSACKTIEDALRNSARLESADVSEMLTFDKKAFTLANVSSFGDYNLSSVIPSLPTSGSRVAGRSAIEDILFSKLVTSGLGTVDADYKKCTKGLSIADLACAQYYNGIMVLPGVADAERMAMYTGSLIGGIALGGLTSAVSIPFSLAIQARLNYVALQTDVLQENQKILAASFNKAMTNIVDAFTGVNDAITQTSQALQTVATALNKIQDVVNQQGNSLNHLTSQLRQNFQAISSSIQAIYDRLDTIQADQQVDRLITGRLAALNVFVSHTLTKYTEVRASRQLAQQKVNECVKSQSKRYGFCGNGTHIFSIVNAAPEGLVFLHTVLLPTQYKDVEAWSGLCVDGTNGYVLRQPNLALYKEGNYYRITSRIMFEPRIPTMADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWWVWLCISVVLIFVVSMLLLCCCSTGCCGFFSCFASSIRGCCESTKLPYYDVEKIHIQ

SEQ ID NO: 8 - protein, 1356 aa, Betacoronavirus
  HCOV-NL63 Spike protein full-length
MKLFLILLVLPLASCFFTCNSNANLSMLQLGVPDNSSTIVTGLLPTHWFCANQSTSVYSANGFFYIDVGNHRSAFALHTGYYDANQYYIYVTNEIGLNASVTLKICKFSRNTTFDFLSNASSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVVDVMRYNLNFSANSLDNLKSGVIVFKTLQYDVLFYCSNSSSGVLDTTIPFGPSSQPYYCFINSTINTTHVSTFVGILPPTVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDSIQADQQVDRLITGRLAALNAFVSQVLNKYTEVRGSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENYIKWPWWVWLIISVVFVVLLSLLVFCCLSTGCCGCCNCLTSSMRGCCDCGSTKLPYYEFEKVHVQ

SEQ ID NO: 9 - protein, 114 aa, SARS coronavirus
  SARS-CoV-2 Spike protein RBD domain
SETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQI

SEQ ID NO: 10 - protein, 114 aa, SARS coronavirus
  SARS-CoV Spike protein RBD domain
AELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQI

SEQ ID NO: 11 - protein, 112 aa, Betacoronavirus
  MERS-CoV Spike protein RBD domain
SQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDL

SEQ ID NO: 12 - protein, 114 aa, Betacoronavirus
  HCoV-OC43 Spike protein RBD domain
SEIKCKTQSIAPPTGVYELNGYTVQPIADVYRRKPDLPNCNIEAWLNDKSVPSPLNWERKTFSNCNFNMSSLMSFIQADSFTCNNIDAAKIYGMCFSSITIDKFAIPNGRKVDL

SEQ ID NO: 13 - protein, 114 aa, Betacoronavirus
  HCoV-HKU1 Spike protein RBD domain
SEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDL

SEQ ID NO: 14 - protein, 80 aa, Betacoronavirus
  HCoV-229E Spike protein RBD domain
NRLRCDQLSFDVPDGFYSTSPIQSVELPVSIVSLPVYHKHTFIVLYVDFKPQSGGGKCFNCYPAGVNITLANFNETKGPL

SEQ ID NO: 15 - protein, 73 aa, Betacoronavirus
  HCoV-NL63 Spike protein RBD domain
EKLQCEHLQFGLQDGFYSANFLDDNVLPETYVALPIYYQHTDINFTATASFGGSCYVCKPHQVNISLNGNTSV

SEQ ID NO: 16 - protein, 78 aa, Artificial Sequence
  SITE 1..1: any amino acid
  Consensus sequence
  SITE 2..2: any amino acid
  SITE 3..3: amino acid having a hydrophobic side chain
  SITE 4..4: any amino acid
  SITE 5..5: amino acid having a hydrophobic side chain
  SITE 6..6: any amino acid
  SITE 7..7: amino acid having a hydrophobic side chain
  SITE 8..8: any amino acid
  SITE 9..9: any amino acid
  SITE 10..10: amino acid having a hydrophobic side chainamino acid having a hydrophobic side chain
  SITE 11..11: amino acid having a hydrophobic side chain
  SITE 12..12: any amino acid
  SITE 13..13: any amino acid
  SITE 14..14: any amino acid
  SITE 15..15: any amino acid
  SITE 16..16: any amino acid
  SITE 17..17: amino acid having a hydrophobic side chain
  SITE 18..18: any amino acid
  SITE 19..19: any amino acid
  SITE 20..20: any amino acid
  SITE 21..21: any amino acid
  SITE 22..22: any amino acid
  SITE 23..23: any amino acid
  SITE 24..24: any amino acid
  SITE 25..25: any amino acid
  SITE 26..26: amino acid having a hydrophobic side chain
  SITE 27..27: any amino acid
  SITE 28..28: any amino acid
  SITE 29..29: amino acid having a hydrophobic side chain
  SITE 30..30: any amino acid
  SITE 31..31: amino acid having a hydrophobic side chain
  SITE 32..32: any amino acid
  SITE 34..34: any amino acid
  SITE 35..35: any amino acid
  SITE 36..36: amino acid having a hydrophobic side chain
  SITE 38..38: any amino acid
  SITE 39..39: any amino acid
  SITE 40..40: any amino acid
  SITE 41..41: any amino acid
  SITE 42..42: amino acid having a hydrophobic side chain
  SITE 43..43: any amino acid
  SITE 44..44: any amino acid
  SITE 45..45: amino acid having a hydrophobic side chain
  SITE 46..46: any amino acid
  SITE 47..47: amino acid having a hydrophobic side chain
  SITE 48..48: any amino acid
  SITE 49..49: any amino acid
  SITE 50..50: any amino acid
  SITE 51..51: any amino acid
  SITE 52..52: any amino acid
  SITE 53..53: any amino acid
  SITE 54..54: any amino acid
  SITE 55..55: amino acid having a hydrophobic side chain
  SITE 56..56: any amino acid
  SITE 57..57: any amino acid
  SITE 58..58: any amino acid
  SITE 59..59: any amino acid
  SITE 60..60: amino acid having a hydrophobic side chain
  SITE 61..61: any amino acid
  SITE 62..62: any amino acid
  SITE 63..63: any amino acid
  SITE 64..64: any amino acid
  SITE 65..65: any amino acid
  SITE 66..66: any amino acid
  SITE 67..67: any amino acid
  SITE 68..68: any amino acid
  SITE 69..69: any amino acid
  SITE 70..70: any amino acid
  SITE 71..71: any amino acid
  SITE 72..72: any amino acid
  SITE 73..73: any amino acid
  SITE 74..74: any amino acid
  SITE 75..75: any amino acid
  SITE 78..78: any amino acid
XXXXXXXXXXXXXXXXXXXXXXXXXXXXXXXXYXXXYXXXXXXXXXXXXXXXXXXXXXXXXXXXXXXXXXXXXXXRQX

SEQ ID NO: 17 - protein, 77 aa, SARS coronavirus
  LA binding pocket of SARS-CoV 2
NLCPFGEVFNATRFASVYAWNRKRISNCVADYSVLYNSASFSTFKCYGVSPTKLNDLCFTNVYADSFVIRGDEVRQI

SEQ ID NO: 18 - protein, 77 aa, SARS coronavirus
  LA binding pocket of SARS-CoV
NLCPFGEVFNATKFPSVYAWERKKISNCVADYSVLYNSTFFSTFKCYGVSATKLNDLCFSNVYADSFVVKGDDVRQI